(19) United States
(12) Patent Application Publication
(10) Pub. No.: US 2010/0093668 A1
(43) Pub. Date: Apr. 15, 2010

(54) N,N'-2,4-DIANILINOPYRIMIDINE DERIVATIVES, PREPARATION THEREOF AS DRUGS, PHARMACEUTICAL COMPOSITIONS ESSENTIALLY AS IKK INHIBITORS

(75) Inventors: Didier BABIN, Montigny (FR); Monsif BOUABOULA, Juvignac (FR); Pierre CASELLAS, Castelnau-le-lez (FR); Maria MENDEZ-PEREZ, Francfort (DE); Serge MIGNANI, Chatenay-Malabry (FR); Jean-Flaubert NGUEFACK, Lattes (FR); Jacob-Alsboek OLSEN, Francfort (DE); Bernard TONNERRE, Vailhauques (FR); Jean WAGNON, Montpellier (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/495,998

(22) Filed: Jul. 1, 2009

Related U.S. Application Data
(63) Continuation of application No. PCT/FR2008/000002, filed on Jan. 2, 2008.

(30) Foreign Application Priority Data
Jan. 5, 2007 (FR) 0700064

(51) Int. Cl. C07D 401/12; A61K 31/506; A61K 31/675; A61K 31/497; C07D 403/12; C07D 413/12; A61K 31/5355; C12N 9/99; A61P 35/04

(52) U.S. Cl. 514/85; 514/275; 544/297; 514/255.05; 544/295; 544/122; 514/235.8; 514/210.2; 435/184

(57) ABSTRACT

The disclosure relates to a compound of formula (I):

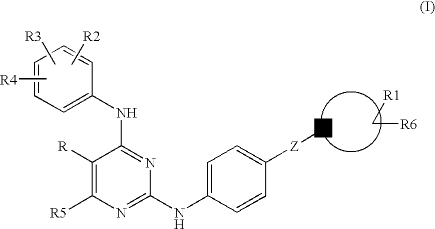

wherein R, R1, R2, R3, R4, R5, R6 and Z are as defined in the specification, to compositions containing them, to processes for preparing them, and to their use in the treatment or prevention of conditions capable of being modulated by the inhibition of the activity of protein kinases.

This application is a continuation of International Application No. PCT/FR2008/000002, filed Jan. 2, 2008, which is incorporated herein by reference in its entirety; which claims the benefit of priority of French Patent Application No. 0700064, filed Jan. 5, 2007.

The present invention relates to novel N,N′-2,4-dianilinopyrimidine derivatives, the process for their preparation, the novel intermediates obtained, their application as medicaments, the pharmaceutical compositions containing them and the novel use of such 2,4-dianilinopyrimidine derivatives.

Patent WO 2001/64654 A1 mentions 2,4-di(hetero)arylpyrimidines substituted at the 5-position and which are inhibitors of the kinases CDK2 and FAK. Likewise, other aminopyrimidines which are inhibitors of serine-threonine kinases and of CDK are presented in WO 2003/030909 A1. Patent WO 2004/046118 A2 describes 2,4-diphenylaminopyrimidine derivatives as cell proliferation inhibitors.

A series of 5-cyano-2-aminopyrimidines are presented as inhibitors of the kinases KDR and FGFR, in WO 2000/78731 A1, other pyrimidines as inhibitors of FAK and IGFR in WO 2004/080980 A1 and also of ZAP-70, FAK and/or Syk tyrosine kinase in WO 2003/078404 A1, and polokinases PLK in WO 2004/074244 A2, as cytostatic agents.

Likewise, other patents describe reverse transcriptase inhibiting pyrimidines for the treatment of HIV-related infections (WO 2001/85700 A2; WO 2001/85699 A2; WO 2000/27825 A1 and WO 2003/094920 A1).

The subject of the present invention is thus novel 2,4-dianilinopyrimidine derivatives having inhibitory effects on protein kinases.

The products of the present invention may thus in particular be used for the prevention or treatment of conditions capable of being modulated by the inhibition of the activity of protein kinases.

Among these protein kinases, there may be mentioned more particularly protein kinase IKK-alpha (IKKα) and IKK-beta (IKKβ).

The compounds of the present invention are kinase inhibitors, in particular IKK-alpha and IKK-beta inhibitors, and consequently inhibit the NF-KB (nuclear factor kappa B) activity; they may thus be used in the treatment or the prophylaxis of inflammatory diseases, in cancer and diabetes.

NF-kB (Nuclear factor kappa B) belongs to a family of complexes of transcription factors consisting of various combinations of polypeptides Rel/NF-KB. Members of this family of polypeptides linked to NF-KB regulate the expression of genes involved in immune and inflammatory responses ((Bames P J, Karin M (1997) N Engl J Med 336, 1066-1071) and (Baeuerle P A, Baichwal V R (1997) Adv Immunol 65, 111-137)). Under basal conditions, the NF-KB dimers are retained in the inactive form in the cytoplasm by inhibitory proteins which are members of the IKB family (Beg et al., Genes Dev., 7:2064-2070, 1993; Gilmore and Morin, Trends Genet. 9:427-433), 1993); Haskil et al., Cell 65: 1281-1289, 1991). The proteins of the IKB family mask the signal for nuclear translocation of NF-KB. The stimulation of the cell by various types of ligands such as cytokines, the anti-CD40 ligand, lipopolysaccharides (LPS), oxidants, mitogens such as phorbol ester, viruses and many other stimulants, causes the activation of the IKB-kinase (IKK) complex which will in turn phosphorylate IKB at the level of the serine 32 and 34 residues. Once phosphorylated, IKB will be subject to ubiquitinations leading to its degradation by the proteasome (26S), thus allowing the release and translocation of NF-KB in the nucleus where it will bind to specific sequences at the level of the target gene promoters, thus inducing their transcription.

In the IKB-kinase (IKK) complex, the principal kinases are IKK1 (IKKα) and IKK2 (IKKβ) which are capable of directly phosphorylating the various classes of IKB. In this IKK complex, IKK2 is the dominant kinase (Mercurio et al., Mol. Cell Biol., 19:1526, 1999-, Zandi et al., Science; 28 1: 1 3) 60, 1998; Lee et al., Proc. Natl. Acad. Sci. USA 95:93) 19, 1998).

Among the genes regulated by NF-KB, many encode pro-inflammatory mediators, cytokines, cell adhesion molecules, acute phase proteins, which will in turn induce the activation of NF-KB by autocrine or paracrine mechanisms.

The inhibition of the activation of NF-KB appears to be very important in the treatment of inflammatory diseases.

In addition, NF-KB plays a role in the growth of normal cells but also of malignant cells.

The proteins produced by the expression of genes regulated by NF-KB comprise cytokines, chemokines, adhesion molecules, mediators of cell growth, of angiogenesis. Moreover, various studies have shown that NF-KB plays an essential role in neoplastic transformations. For example, NF-KB may be associated with the transformation of cells in vitro and in vivo following over expression, amplification, rearrangement or translocation events (Mercurio, R, and Manning, A. M. (1999) Oncogene, 18: 6163-6171). In some human lymphoid tumor cells, the genes encoding the various NF-KB members are rearranged or amplified. It has been shown that NF-KB may promote cell growth by inducing the transcription of cyclin D, which, when associated with the hyperphosphorylation of Rb, causes the G1 to S phase transition and the inhibition of apoptosis.

It has been shown that in a large number of tumor cell lines, a constitutive NF-KB activity is present following the activation of IKK2. NF-KB is constitutively activated in Hodgkin's diseases and the inhibition of NF-KB blocks the growth of these lymphomas. Moreover, the inhibition of NF-KB by the expression of the IKBa repressor induces the apoptosis of cells expressing the oncogenic allele H-Ras (Baldwin, J. Clin. Invest., 107:241 (2001), Bargou et al., J. Clin. Invest., 100:2961 (1997), Mayo et al., Science 178:1812 (1997).

The constitutive activity of NF-KB appears to contribute to oncogenesis through the activation of several anti-apoptotic genes such as Al/Bfi-1, IEX-1, MAP, which thus causes the suppression of the cell death pathway. Through the activation of cyclin D, NF-KB may promote the growth of tumor cells. The regulation of adhesion molecules and of surface proteases suggest a role for NF-KB signaling in metastases.

NF-KB is involved in the induction of chemoresistance. NF-KB is activated in response to a certain number of chemotherapy treatments. It has been shown that the inhibition of NF-KB by the use of the superrepressor form of IKBa in parallel with the chemotherapy treatment increases the efficacy of the chemotherapy in the xenograft models.

The subject of the present invention is in particular the products of formula (I):

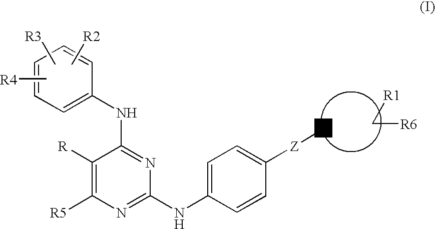

in which:

R represents a hydrogen or halogen atom,

R2, R3 and R4, which may be identical or different, are such that one represents a halogen atom or CF₃ and the other two, which may be identical or different, represent a hydrogen atom or a halogen atom or an alkyl radical or an alkoxy radical optionally substituted with one or more halogen atoms;

R5 represents a hydrogen atom or a halogen atom;

Z represents CO or SO₂;

the ring (N), i.e.

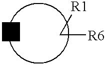

being substituted on the same carbon atom with R1 and R6, containing 4 to 7 ring members, being saturated and possibly also containing a carbon-based bridge consisting of 1 to 3 carbons,

it being understood that R1 and R6 represent one of the following 6 alternatives i) to vi):

i) R1 represents —X1-R7 with X1 representing —(CH₂)ₘ— and R7 representing a heterocycloalkyl, aryl or heteroaryl ring, all optionally substituted;

and R6 represents a hydrogen atom, or the radicals hydroxyl, methyl, methoxy, —(CH₂)ₘOH, —CO—NRaRb, —CH₂—NraRb, —CO₂H and —CO₂alk;

ii) R1 represents —X2-R7 with X2 representing:

—O—; —O—(CH₂)ₘ—; —CH(OH)—(CH₂)ₙ—; —CO—; —CO—NRc-;

—CO—NRc-O—; —CH(NRaRb)-; —C═NOH—; —C═N—NH₂—; —(CH₂)ₙ₁—NRc-(CH₂)ₙ₂—; and R7 represents a heterocycloalkyl, aryl or heteroaryl ring, all optionally substituted;

and R6 represents hydrogen or the methyl radical;

iii) R1 represents —NRc-W with W representing a hydrogen atom or an alkyl radical containing from 1 to 4 carbon atoms which is linear or branched from 3 carbon atoms and optionally substituted with a radical chosen from —PO(OEt)₂, —OH, —Oalk, —CF₃, —CO—NR8R9 and SO₂-alk; and R6 represents hydrogen;

it being understood that, when W represents a hydrogen atom, then z represents CO;

iv) R1 represents —CH₂—NRc-W with W representing a hydrogen atom or an alkyl radical containing from 1 to 4 carbon atoms which is linear or branched from 3 carbon atoms and optionally substituted with a radical chosen from —PO(OEt)₂, —OH, —OEt, —CF₃, —CO—N(alk)₂ and SO₂-alk; and R6 represents hydrogen;

v) R1 represents —CO—N(Rc)-OR′c and R6 represents hydrogen;

iv) R1 represents X3-R7 with X3 representing —CH(OH)—(CH₂)ₙ—; —CO—; —CH(NRaRb)—; —C═NOH—; —C═N—NH₂—;

and R7 represents a heterocycloalkyl, aryl or heteroaryl ring, all optionally substituted;

and R6 represents a hydrogen atom or the radicals hydroxyl, methyl, methoxy, —(CH₂)ₘOH, —CO—NRaRb, —CH₂—NRaRb and —CO₂alk;

with n, n1 and n2, which may be identical or different, representing an integer from 0 to 3;

m represents an integer from 1 to 3;

Rc and R′c, which may be identical or different, represent a hydrogen atom or an alkyl radical containing from 1 to 4 carbon atoms, optionally substituted with one or more halogen atoms;

NRaRb is such that either Ra and Rb, which may be identical or different, represent a hydrogen atom or an alkyl radical containing from 1 to 4 carbon atoms or a cycloalkyl radical, these alkyl and cycloalkyl radicals being optionally substituted with one or more halogen atoms, a hydroxyl radical or an NH₂, NHalkyl or N(alkyl)₂ radical; or Ra and Rb form, with the nitrogen atom to which they are attached, a cyclic amine that may optionally contain one or two other heteroatoms chosen from O, S, N or NR10, the cyclic amine thus formed itself being optionally substituted with one or more radicals, which may be identical or different, chosen from halogen atoms and the radicals oxo; hydroxyl; alkyl, themselves optionally substituted with one or more halogen atoms, or else with a methyl radical or a hydroxyl radical on the same carbon;

all the heterocycloalkyl, aryl and heteroaryl radicals being optionally substituted with one or more radicals, which may be identical or different, chosen from halogen atoms; hydroxyl, cyano or NR8R9 radicals; and the radicals alkyl, cycloalkyl, alkoxy, phenyl, heterocycloalkyl and heteroaryl, themselves optionally substituted with one or more radicals, which may be identical or different, chosen from halogen atoms and the radicals hydroxyl, alkoxy, alkyl, hydroxyalkyl, alkoxyalkyl, CN, CF₃, OCF₃ or NRaRb;

NR8R9 is such that either R8 and R9, which may be identical or different, are such that R8 represents a hydrogen atom or an alkyl radical containing from 1 to 4 carbon atoms or a cycloalkyl radical, these alkyl and cycloalkyl radicals being optionally substituted with one or more halogen atoms, a hydroxyl radical or an NH₂, NHalkyl or N(alkyl)₂ radical; and

R9 represents a hydrogen atom or the radicals alkyl, cycloalkyl or heterocycloalkyl, themselves optionally substituted with one or more radicals, which may be identical or different, chosen from halogen atoms and the radicals hydroxyl, alkoxy, NH₂, NHalkyl or N(alkyl)₂, the alkyl radicals represented by R9 also being optionally substituted with a phenyl, heterocycloalkyl or heteroaryl radical, itself optionally substituted with one or more radicals chosen from halogen atoms and the radicals hydroxyl, alkoxy, alkyl, hydroxyalkyl, alkoxyalkyl, CN, CF₃, OCF₃, NH₂, NHalk or N(alk)₂;

or R8 and R9 form, with the nitrogen atom to which they are attached, a cyclic amine that may optionally contain one or two other heteroatoms chosen from O, S, N or NR10, the cyclic amine thus formed itself being optionally substituted with one or more radicals, which may be identical or different, chosen from halogen atoms and alkyl radicals, themselves optionally substituted with one or more halogen atoms;

all the above heterocycloalkyl and heteroaryl radicals, optionally substituted as indicated above, consisting of from 4 to 10 ring members and containing from 1 to 3 heteroatoms chosen from O, S, N and NR10;

R10 represents a hydrogen atom or an alkyl radical, said products of formula (I) being in all the possible racemic, enantiomeric and diastereoisomeric isomer forms, and also the addition salts with inorganic and organic acids of said products of formula (I).

A subject of the present invention is in particular the products of formula (I) as defined above in which R2, R3, R4, R5, Z and the ring (N) and also R1 and R6 have the meanings indicated above or hereinafter and R represents a halogen atom;

said products of formula (I) being in all the possible racemic, enantiomeric and diastereoisomeric isomer forms, and also the addition salts with inorganic and organic acids of said products of formula (I).

A subject of the present invention is the products of formula (I) as defined above or hereinafter in which R2, R3, R4, R5, Z and the ring (N) and also R1 and R6 have the meanings indicated in any one of the other claims and R represents a hydrogen atom;

said products of formula (I) being in all the possible racemic, enantiomeric and diastereoisomeric isomer forms, and also the addition salts with inorganic and organic acids of said products of formula (I).

A subject of the present invention is thus the products of formula (I) as defined above in which R has the meaning indicated above or hereinafter, R2, R3 and R4, which may be identical or different, are such that one represents a halogen atom or CF₃ and the other two, which may be identical or different, represent a hydrogen atom or a halogen atom or an alkyl radical or an alkoxy radical optionally substituted with one or more halogen atoms;

R5 represents a hydrogen atom or a halogen atom;

Z represents CO or SO₂;

the ring (N), i.e.

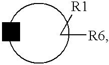

being substituted on the same carbon atom with R1 and R6, containing 4 to 7 ring members, being saturated and possibly also containing a carbon-based bridge consisting of from 1 to 3 carbons,

it being understood that R1 and R6 represent one of the 5 following alternatives i) to v):

i) R1 represents —X1-R7 with X1 representing —(CH₂)ₘ— and R7 represents a heterocycloalkyl, aryl or heteroaryl ring, all optionally substituted;

and R6 represents a hydrogen atom or the radicals hydroxyl, —(CH₂)ₘOH, —CO—NRaRb, —CH₂—NRaRb, —CO₂H and —CO₂alk;

ii) R1 represents —X2-R7 with X2 representing:

—O—; —O—(CH₂)ₘ—; —CH(OH)—(CH₂)ₙ—; —CO—; —CO—NRc-; —CO—NRc-O—; —CH(NRaRb)—; —C═NOH—; —C═N—NH₂—; —(CH₂)ₙ₁—NRc-(CH₂)ₙ₂—; and R7 represents a heterocycloalkyl, aryl or heteroaryl ring, all optionally substituted;

and R6 represents hydrogen;

iii) R1 represents —NRc-W with W representing a hydrogen atom or an alkyl radical containing from 1 to 4 carbon atoms which is linear or branched from 3 carbon atoms and optionally substituted with a radical chosen from —PO(OEt)₂, —OH, —Oalk, —CF₃, —CO—NR8R9 and SO₂-alk; and R6 represents hydrogen; it being understood that, when W represents a hydrogen atom, then z represents CO;

iv) R1 represents —CH₂—NRc-W with W representing a hydrogen atom or an alkyl radical containing from 1 to 4 carbon atoms which is linear or branched from 3 carbon atoms and optionally substituted with a radical chosen from —PO(OEt)₂, —OH, —OEt, —CF₃, —CO—N(alk)₂ and SO₂-alk; and R6 represents hydrogen;

v) R1 represents —CO—N(Rc)-OR′c and R6 represents hydrogen;

with n, n1 and n2, which may be identical or different, representing an integer from 0 to 3;

m represents an integer from 1 to 3;

Rc and R′c, which may be identical or different, represent a hydrogen atom or an alkyl radical containing from 1 to 4 carbon atoms, optionally substituted with one or more halogen atoms, it being understood that the halogen atoms are not in the vicinal position with respect to the nitrogen atom;

NRaRb is such that either Ra and Rb, which may be identical or different, represent a hydrogen atom or an alkyl radical containing from 1 to 4 carbon atoms or a cycloalkyl radical, these alkyl and cycloalkyl radicals being optionally substituted with one or more halogen atoms, it being understood that the halogen atoms are not in the vicinal position with respect to the nitrogen atom; a hydroxyl radical or an NH₂, NHalkyl or N(alkyl)₂ radical; or Ra and Rb form, with the nitrogen atom to which they are attached, a cyclic amine that may optionally contain one or two other heteroatoms chosen from O, S, N or NR10, the cyclic amine thus formed itself being optionally substituted with one or more radicals, which may be identical or different, chosen from halogen atoms and alkyl radicals, themselves optionally substituted with one or more halogen atoms;

all the heterocycloalkyl, aryl and heteroaryl radicals being optionally substituted with one or more radicals, which may be identical or different, chosen from halogen atoms; hydroxyl; cyano or NR8R9 radicals; and the radicals alkyl, cycloalkyl, alkoxy, phenyl, heterocycloalkyl and heteroaryl, themselves optionally substituted with one or more radicals, which may be identical or different, chosen from halogen atoms and the radicals hydroxyl, alkoxy, alkyl, hydroxyalkyl, alkoxyalkyl, CN, CF₃, OCF₃ or NRaRb;

NR8R9 is such that either R8 and R9, which may be identical or different, are such that R8 represents a hydrogen atom or an alkyl radical containing from 1 to 4 carbon atoms or a cycloalkyl radical, these alkyl and cycloalkyl radicals being optionally substituted with one or more halogen atoms, a hydroxyl radical or an NH₂, NHalkyl or N(alkyl)₂ radical; and R9 represents a hydrogen atom or the radicals alkyl, cycloalkyl or heterocycloalkyl, themselves optionally substituted with one or more radicals, which may be identical or different, chosen from halogen atoms and the radicals hydroxyl, alkoxy, NH₂, NHalkyl or N(alkyl)₂, the alkyl radicals represented by R9 also being optionally substituted with a phenyl, heterocycloalkyl or heteroaryl radical, itself optionally substituted with one or more radicals chosen from halogen atoms and the radicals hydroxyl, alkoxy, alkyl, hydroxyalkyl, alkoxyalkyl, CN, CF₃, OCF₃, NH₂, NHalk or N(alk)₂;

or R8 and R9 form, with the nitrogen atom to which they are attached, a cyclic amine that may optionally contain one or two other heteroatoms chosen from O, S, N or NR10, the cyclic amine thus formed itself being optionally substituted with one or more radicals, which may be identical or different, chosen from halogen atoms and alkyl radicals, themselves optionally substituted with one or more halogen atoms;

all the above heterocycloalkyl and heteroaryl radicals optionally substituted as indicated above, consisting of from 4 to 10 ring members and containing 1 to 3 heteroatoms chosen from O, S, N and NR10;

R10 represents a hydrogen atom or an alkyl radical,

said products of formula (I) being in all the possible racemic, enantiomeric and diastereoisomeric isomer forms, and also the addition salts with inorganic and organic acids of said products of formula (I).

A subject of the present invention is thus the products of formula (I) as defined above in which R, R2, R3, R4, R5, Z and the ring (N) have the meanings indicated above or hereinafter and R1 and R6 are such that R1 represents —X1-R7 with X1 representing —(CH₂)ₘ— and R7 represents a heterocycloalkyl, aryl or heteroaryl ring, all optionally substituted;

and R6 represents a hydrogen atom or the radicals hydroxyl, —(CH₂)ₘOH, —CO—NRaRb, —CH₂—NRaRb, —CO₂H, and —CO₂alk;

with m, n and NRaRb as defined above or hereinafter and the heterocycloalkyl, aryl and heteroaryl radicals being optionally substituted with one or more radicals, which may be identical or different, as defined above or hereinafter,

said products of formula (I) being in all the possible racemic, enantiomeric and diastereoisomeric isomer forms, and also the addition salts with inorganic and organic acids of said products of formula (I).

A subject of the present invention is thus the products of formula (I) as defined above in which R, R2, R3, R4, R5, Z and the ring (N) have the meanings indicated above and R1 and R6 are such that R1 represents —X2-R7 with X2 representing:

—O—, —O—(CH₂)ₘ—, —CH(OH)—(CH₂)ₙ—, —CO—, —CO—NRc-, —CO—NRc-O—, —CH(NRaRb)—, —C═NOH—, —C═N—NH₂—, —(CH₂)ₙ₁—NRc-(CH₂)ₙ₂—;

and R7 represents a heterocycloalkyl, aryl or heteroaryl ring, all optionally substituted,

and R6 represents hydrogen;

with n, n1, n2, Rc and NRaRb as defined above and the heterocycloalkyl, aryl and heteroaryl radicals being optionally substituted with one or more radicals, which may be identical or different, as defined above or hereinafter,

said products of formula (I) being in all the possible racemic, enantiomeric and diastereoisomeric isomer forms, and also the addition salts with inorganic and organic acids of said products of formula (I).

A subject of the present invention is the products of formula (I) as defined above in which R, R2, R3, R4, R5, Z and the ring (N) have the meanings indicated above or hereinafter and R1 and R6 are such that:

either R1 represents —NRc-W with W representing a hydrogen atom or an alkyl radical containing from 1 to 4 carbon atoms which is linear or branched from 3 carbon atoms and optionally substituted with a radical chosen from —PO(OEt)₂, —OH, —Oalk, —CF₃, —CO—NR8R9 and SO₂-alk, and R6 represents hydrogen, it being understood that, when W represents a hydrogen atom, then z represents CO;

or R1 represents —CH₂—NRc-W with W representing a hydrogen atom or an alkyl radical containing from 1 to 4 carbon atoms which is linear or branched from 3 carbon atoms and optionally substituted with a radical chosen from —PO(OEt)₂, —OH, —OEt, —CF₃, —CO—N(alk)₂ and SO₂-alk;

and R6 represents hydrogen;

or R1 represents —CO—N(Rc)-OR′c and R6 represents hydrogen; with Rc, R′c and NR8R9 as defined above,

said products of formula (I) being in all the possible racemic, enantiomeric and diastereoisomeric isomer forms, and also the addition salts with inorganic and organic acids of said products of formula (I).

When the products of formula (I) are such that R, R2, R3, R4, R5 and the ring (N) have the meanings indicated above or hereinafter, z represents SO₂ and R1 and R6 are such that R1 represents —NRc-W with Rc as defined above and R6 represents hydrogen, then the present invention relates in particular to the products in which W represents an alkyl radical containing from 1 to 4 carbon atoms which is linear or branched from 3 carbon atoms and substituted with a radical chosen from —PO(OEt)₂, —OH, —Oalk, —CF₃, —CO—NR8R9 and SO₂-alk;

said products of formula (I) being in all the possible racemic, enantiomeric and diastereoisomeric isomer forms, and also the addition salts with inorganic and organic acids of said products of formula (I).

When the ring (N) contains a carbon-based bridge consisting of from 1 to 3 carbons, the ring formed may in particular be the 8-azabicyclo(3,2,1)oct-3-yl ring or else a ring chosen from the following: azabicyclo[3.3.1]nonan-3-yl, 6-azabicyclo[3.2.1]octan-3-yl, 3-azabicyclo[3.2.1]octan-8-yl or else 3-azabicyclo[3.3.1]nonan-9-yl.

A subject of the present invention is thus the products of formula (I) as defined above in which R2, R3, R4, R5 and Z have the meanings indicated above or hereinafter and the ring (N) represents one of the rings defined hereinafter:
an azetidinyl or pyrrolidinyl ring substituted in the 3 position with R1 and R6 as defined above or hereinafter;
a piperidinyl and azepinyl ring substituted in the 3- or 4-position with R1 and R6 as defined above or hereinafter;
an 8-azabicyclo(3,2,1)octan-3-yl, 6-azabicyclo[3.2.1]octan-3-yl or 3-azabicyclo[3.2.1]octan-8-yl ring;

said products of formula (I) being in all the possible racemic, enantiomeric and diastereoisomeric isomer forms, and also the addition salts with inorganic and organic acids of said products of formula (I).

A subject of the present invention is thus the products of formula (I) as defined above in which R, R2, R3, R4, R5 and Z have the meanings indicated above or hereinafter and the ring (N) represents a pyrrolidinyl ring substituted in the 3-position with R1 and R6 as defined above or hereinafter or a piperidinyl ring substituted in the 3- or 4-position with R1 and R6 as defined above or hereinafter,

said products of formula (I) being in all the possible racemic, enantiomeric and diastereoisomeric isomer forms, and also the addition salts with inorganic and organic acids of said products of formula (I).

In the products of formula (I) and in the subsequent text, the terms indicated have the meanings which follow:
the term “halogen” denotes fluorine, chlorine, bromine or iodine atoms and preferably fluorine, chlorine or bromine atoms;
the term “alkyl radical” denotes a linear or branched radical containing at most 6 carbon atoms, and in particular methyl, ethyl, propyl, isopropyl, n-butyl, isobutyl, sec-butyl, tert-butyl, pentyl, isopentyl, sec-pentyl, tert-pentyl, neopentyl, hexyl, isohexyl, sec-hexyl and tert-hexyl radicals, and also the linear or branched positional isomers thereof;
the term “hydroxyalkyl radical” denotes the alkyl radicals indicated above, substituted with one or more hydroxyl radicals;
the term “alkoxy radical” denotes a linear or branched radical containing at most 6 carbon atoms, chosen for example from methoxy, ethoxy, propoxy, isopropoxy, linear, secondary or tertiary butoxy, pentoxy, hexoxy and heptoxy radicals, and also the linear or branched positional isomers thereof;
the term “cycloalkyl radical” denotes a monocyclic or bicyclic carbocyclic radical containing from 3 to 7 ring members and denotes in particular cyclopropyl, cyclobutyl, cyclopentyl, cyclohexyl and cycloheptyl radicals;
the term “aryl radical” denotes unsaturated carbocyclic radicals that are monocyclic or constituted of fused rings. As examples of such an aryl radical, mention may in particular be made of phenyl or naphthyl radicals;
the term “heterocyclic radical” denotes a saturated carbocyclic (heterocycloalkyl) or partially or completely unsaturated carbocyclic (heteroaryl) radical constituted of from 4 to 10 ring members interrupted with one to four heteroatoms, which may be identical or different, chosen from oxygen, nitrogen or sulphur atoms;

among the heteroaryl radicals with 5 ring members, mention may in particular be made of radicals containing one to four heteroatoms chosen from N which is optionally oxidized, O and S which is optionally oxidized, as such radicals mention may be made of the radicals thienyl, such as 2-thienyl, 3-thienyl or dioxidothienyl, triazolyl (N,S), furyl (O), 2-furyl, pyrrolyl (NH, NCH₃), isothiazolyl, diazolyl, thiadiazolyl (N,N,S), 1,3,4-thiadiazolyl, oxazolyl, oxadiazolyl, isoxazolyl (N,O), 3-isoxazolyl, 4-isoxazolyl, imidazolyl, pyrazolyl (N,N), triazolyl and tetrazolyl groups, and more particularly oxazolyl, isoxazolyl (N,O) or pyrazolyl radicals; all these rings being optionally substituted with one or more radicals as defined above or hereinafter, these substituents of course being at the positions chemically acceptable for each of these rings;

among the heteroaryl radicals with 6 ring members, mention may in particular be made of pyridyl radicals, such as 2-pyridyl, 3-pyridyl and 4-pyridyl, pyridyl-N-oxide radicals, pyrimidinyl radicals, pyridazinyl radicals and pyrazinyl radicals;

among the fused heteroaryl radicals containing at least one heteroatom chosen from sulphur, nitrogen and oxygen, mention may, for example, be made of the radicals benzothienyl, benzofuryl, benzofuranyl, benzoxazolyl, indazolyl, indolyl, indolinyl, indolinonyl, quinolyl, isoquinolyl, azaindolyl, benzimidazolyl, benzothiazolyl, naphthyridinyl such as [1,8]naphthyridinyl; imidazo(4,5)pyridinyl; indolizinyl; quinazolinyl; 2,3-dihydro-1H-indolyl; 2,3-dihydrobenzofuranyl; 4-[(benzo[1,2,5]oxadiazolyl; (2,3-dihydrobenzofuranyl;

among the fused heterocyclic radicals, mention may more particularly be made of the radicals benzothienyl, benzofuranyl, benzodihydrofuranyl, indolyl, indolinyl, indolinonyl, benzimidazolyl, benzothiazolyl, benzoxodiazolyl, benzothiodiazolyl, naphthyridinyl, indazolyl, quinolyl such as 4-quinolyl or 5-quinolyl, isoquinolyl, azaindolyl such as 4-azaindolyl or 3-azaindolyl, imidazo(4,5)pyridyl, indolizinyl, quinazolinyl.

As (saturated) heterocycloalkyl, mention may, for example, be made of the radicals oxiranyl, oxetanyl, tetrahydrofuranyl, dioxolanyl, dithiolanyl, tetrahydropyranyl, dioxanyl, aziridinyl, azetidinyl, pyrrolidinyl, piperidinyl, azepinyl, diazepinyl, piperazinyl, morpholinyl, thiomorpholinyl, dioxidothiomorpholinyl, imidazolidinyl; mention may more particularly be made of pyrrolidinyl, piperidinyl, azepinyl, piperazinyl or morpholinyl radicals;

all the cyclic radicals being optionally substituted as indicated above or hereinafter;
the terms “alkylamino radical” or “NH(alk) radical” and “dialkylamino radical” or “N(alk)₂ radical” thus denote NH₂ amino radicals substituted respectively with one or two linear or branched alkyl radicals, which may be identical or different in the case of dialkylamino, chosen from the alkyl radicals as defined above and optionally substituted as indicated above or hereinafter: mention may, for example, be made of methylamino, ethylamino, propylamino or butylamino radicals, or dimethylamino, diethylamino and methylethylamino radicals;
the term “cycloalkylamino radical” thus denotes an amino radical substituted in particular with a cycloalkyl radical chosen from the radicals defined above; mention may thus, for example, be made of cyclopropylamino, cyclobutylamino, cyclopentylamino or else cyclohexylamino radicals;
the term “cyclic amine” denotes a monocyclic or bicyclic radical containing from 3 to 10 ring members in which at least one carbon atom is replaced with a nitrogen atom, this cyclic radical possibly also containing one or more other heteroatoms chosen from O, S, SO₂, N or NR10 with R10 as defined above; as examples of such cyclic amines, mention may, for example, be made of pyrrolyl, piperidyl, morpholinyl, piperazinyl, pyrrolidinyl and azetidinyl radicals. Mention may more particularly be made of piperidinyl, morpholinyl, piperazinyl, pyrrolidinyl or azetidinyl radicals optionally substituted as indicated above, in particular with an oxo or hydroxyl radical or else hydroxyl and methyl radical on the same carbon.

The term “patient” denotes human beings but also other mammals.

The term “prodrug” denotes a product which can be converted in vivo, by metabolic mechanisms (such as hydrolysis), into a product of formula (I). For example, an ester of a product of formula (I) containing a hydroxyl group can be converted, by hydrolysis in vivo, into its parent molecule.

By way of examples of esters of products of formula (I) containing a hydroxyl group, mention may be made of acetates, citrates, lactates, tartrates, malonates, oxalates, salicylates, propionates, succinates, fumarates, maleates, methylenebis-β-hydroxynaphthoates, gentisates, isethionates, di-p-toluoyl tartrates, methanesulphonates, ethanesulphonates, benzenesulphonates, p-toluenesulphonates, cyclohexylsulphamates and quinates.

Esters of products of formula (I) containing a hydroxyl group that are particularly useful can be prepared from acid residues, such as those described by Bundgaard et. al., J. Med. Chem., 1989, 32, page 2503-2507: these esters include in particular substituted (aminomethyl)benzoates, dialkylaminomethyl benzoates in which the two alkyl groups can be linked together or can be interrupted with an oxygen atom or with a nitrogen atom which is optionally substituted, i.e. an alkylated nitrogen atom, or else morpholinomethyl benzoates, e.g. 3- or 4-(morpholinomethyl)benzoates, and (4-alkylpiperazin-1-yl) benzoates, e.g. 3- or 4-(4-alkylpiperazin-1-yl)benzoates.

When the products of formula (I) comprise an amino radical that can be salified with an acid, it is clearly understood that these acid salts also form part of the invention. Mention may, for example, be made of the salts provided with hydrochloric acid or methanesulphonic acid.

The addition salts with inorganic or organic acids of the products of formula (I) may, for example, be the salts formed with hydrochloric acid, hydrobromic acid, hydriodic acid, nitric acid, sulphuric acid, phosphoric acid, propionic acid, acetic acid, trifluoroacetic acid, formic acid, benzoic acid, maleic acid, fumaric acid, succinic acid, tartric acid, citric acid, oxalic acid, glyoxylic acid, aspartic acid, ascorbic acid, alkoylmonosulphonic acids such as, for example, methanesulphonic acid, ethanesulphonic acid or propanesulphonic acid, alkoyldisulphonic acids such as, for example, methanedisulphonic acid or alpha, beta-ethanedisulphonic acid, arylmonosulphonic acids such as benzenesulphonic acid, and aryldisulphonic acids.

It may be recalled that stereoisomerism can be defined in its broad sense as the isomerism of compounds having the same structural formulae but the various groups of which are arranged differently in space, such as, in particular, monosubstituted cyclohexanes in which the substituent may be in the axial or equatorial position. However, another type of stereoisomerism exists, due to the different spatial arrangements of substituents attached either on double bonds or on rings, which is commonly referred to as E/Z geometric isomerism or cis-trans or diastereoisomeric isomerism. The term “stereoisomer” is used, in the present application, in its broadest sense and therefore involves all the compounds indicated above.

A subject of the present invention is in particular the products of formula (I) as defined above or hereinafter in which:

R has the definition indicated above or hereinafter,

R2, R3 and R4, which may be identical or different, are such that one represents a halogen atom or CF₃ and the other two, which may be identical or different, represent a hydrogen atom or a halogen atom or an alkyl radical or an alkoxy radical optionally substituted with one or more halogen atoms;

R5 represents a hydrogen atom or a halogen atom:

Z represents CO or SO₂;

the ring (N), i.e.

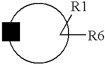

represents a pyrrolidinyl radical substituted in the 3-position with R1 and R6 or a piperidinyl ring substituted in the 3- or 4-position with R1 and R6,

it being understood that R1 and R6 represent one of the following 5 alternatives i) to v):

i) R1 represents —X1-R7 with X1 representing —CH₂ and R7 represents a heterocycloalkyl, phenyl or heteroaryl ring, all optionally substituted;

and R6 represents a hydrogen atom or the radicals hydroxyl, —CH₂OH, —CO₂H, —CO—NRaRb and —CO₂Et;

ii) R1 represents —X2-R7 with X2 representing: —O—, —CH(OH)—, —CH(OH)—CH₂—, —CO—, —CH(NRaRb)—, —C═NOH—, —C═N— NH₂— and —(CH₂)ₙ₁—NRc-(CH₂)ₙ₂—,

and R7 represents a heterocycloalkyl, phenyl or heteroaryl ring, all optionally substituted,

and R6 represents hydrogen;

iii) R1 represents —NRc-W with W representing a hydrogen atom or an alkyl radical containing from 1 to 4 carbon atoms which is linear or branched and optionally substituted with a radical chosen from —PO(OEt)₂, —OH, —OEt, —CF₃, —CO—NR8R9 and SO₂-alk; and R6 represents hydrogen; it being understood that, when W represents a hydrogen atom, then z represents CO;

iv) R1 represents —CH₂—NRc-W with W representing a hydrogen atom or an alkyl radical containing from 1 to 4 carbon atoms which is linear or branched from 3 carbon atoms and optionally substituted with an SO₂-alk radical; and R6 represents hydrogen;

v) R1 represents —CO—N(Rc)-OR′c and R6 represents hydrogen;

with n, n1 and n2, which may be identical or different, representing an integer from 0 to 2;

Rc and R′c, which may be identical or different, represent a hydrogen atom or an alkyl radical containing 1 or 2 carbon atoms;

NRaRb is such that either Ra and Rb, which may be identical or different, represent a hydrogen atom or an alkyl radical containing from 1 to 4 carbon atoms, optionally substituted with one or more halogen atoms, a hydroxyl radical or an NH₂, NHalkyl or N(alkyl)₂ radical; or Ra and Rb form, with the nitrogen atom to which they are attached, a morpholinyl or pyrrolidinyl radical optionally substituted with one or more radicals, which may be identical or different, chosen from halogen atoms and alkyl radicals, themselves optionally substituted with one or more halogen atoms;

all the heterocycloalkyl, phenyl and heteroaryl radicals being optionally substituted with one or more radicals, which may be identical or different, chosen from halogen atoms; the radicals hydroxyl, cyano or NR8R9; and the radicals alkyl, cycloalkyl, alkoxy, phenyl, heterocycloalkyl and heteroaryl, themselves optionally substituted with one or more radicals, which may be identical or different, chosen from halogen atoms and the radicals hydroxyl, alkoxy, OCF₃, CH₃, —CH₂OH, CN, CF₃, OCF₃ or NRaRb;

NR8R9 is such that either R8 and R9, which may identical or different, are such that R8 represents a hydrogen atom, a linear or branched alkyl radical containing at most 4 carbon atoms or a cycloalkyl radical containing from 3 to 6 ring members, alkyl and cycloalkyl themselves optionally substituted with one or more halogen atoms or a hydroxyl radical; and R9 represents a hydrogen atom or an alkyl radical optionally substituted with one or more radicals, which may be identical or different, chosen from halogen atoms, and the radicals hydroxyl, alkoxy, NH₂, NHalkyl, N(alkyl)₂, phenyl, heterocycloalkyl or heteroaryl, themselves optionally substituted with one or more radicals chosen from halogen atoms and the radicals hydroxyl, OCH₃, CH₃, —CH₂OH, CN, CF₃, OCF₃, NH₂, NHalk or N(alk)₂; or R8 and R9 form, with the nitrogen atom to which they are attached, a cyclic amine chosen from pyrrolyl, piperidyl, morpholinyl, pyrrolidinyl, azetidinyl and piperazinyl, optionally substituted with one or more alkyl radicals, themselves optionally substituted with one or more halogen atoms;

said products of formula (I) being in all the possible racemic, enantiomeric and diastereoisomeric isomer forms, and also the addition salts with inorganic and organic acids of said products of formula (I).

It may be noted that a subject of the present invention is in particular the products of formula (I) as defined above or hereinafter in which R, R2, R3, R4, R5 and Z have the meanings indicated above or hereinafter and the ring (N) represents a piperidinyl ring substituted in the 3- or 4-position with R1 and R6 as defined above or hereinafter,

said products of formula (I) being in all the possible racemic, enantiomeric and diastereoisomeric isomer forms, and also the addition salts with inorganic and organic acids of said products of formula (I).

A subject of the present invention is thus the products of formula (I) as defined above in which R, Z, the ring (N), R1 and R6 have the meanings indicated above or hereinafter; R2, R3 and R4, which may be identical or different, are such that one represents a halogen atom or CF₃ and the other two, which may be identical or different, represent a hydrogen atom, a halogen atom or a methyl, methoxy, trifluoromethyl or trifluoromethoxy radical; and R5 represents a hydrogen atom;

said products of formula (I) being in all the possible racemic, enantiomeric and diastereoisomeric isomer forms, and also the addition salts with inorganic and organic acids of said products of formula (I).

A subject of the present invention is thus the products of formula (I) as defined above in which R, Z, the ring (N), R1 and R6 have the meanings indicated above or hereinafter and R2, R3 and R4, which may be identical or different, are such that one represents a fluorine atom and the other two, which may be identical or different, represent a hydrogen atom, a fluorine atom or a methyl radical; R5 represents a hydrogen atom;

said products of formula (I) being in all the possible racemic, enantiomeric and diastereoisomeric isomer forms, and also the addition salts with inorganic and organic acids of said products of formula (I).

A subject of the present invention is thus the products of formula (I) as defined above in which R, R1, R2, R3, R4, R5, R6 and the ring (N) have the meanings indicated above or hereinafter and Z represents SO₂, said products of formula (I) being in all the possible racemic, enantiomeric and diastereoisomeric isomer forms, and also the addition salts with inorganic and organic acids of said products of formula (I).

A subject of the present invention is thus the products of formula (I) as defined above in which R, R1, R2, R3, R4, R5, R6 and the ring (N) have the meanings indicated above or hereinafter and Z represents CO, said products of formula (I) being in all the possible racemic, enantiomeric and diastereoisomeric isomer forms, and also the addition salts with inorganic and organic acids of said products of formula (I).

In the products of formula (I) as defined above, all the heterocycloalkyl, phenyl and heteroaryl radicals represented by R7 may in particular be optionally substituted with one or more radicals, which may be identical or different, chosen from halogen atoms: NR8R9 radicals; and the radicals alkyl, cycloalkyl, alkoxy, phenyl, heterocycloalkyl and heteroaryl, themselves optionally substituted with one or more radicals, which may be identical or different, chosen from halogen atoms and the radicals hydroxyl, alkoxy, OCF₃, CH₃, —CH₂OH, CN, CF₃, OCF₃, NH₂, NHalk or N(alk)₂, pyrrolidinyl, piperidinyl or morpholinyl optionally substituted with one or more radicals, which may be identical or different, chosen from halogen atoms and alkyl radicals, themselves optionally substituted with one or more halogen atoms.

In the products of formula (I) as defined above, NR8R9 may in particular be such that either R8 and R9, which may be identical or different, are such that R8 represents a hydrogen atom, a linear or branched alkyl radical containing at most 4 carbon atoms or a cycloalkyl radical containing from 3 to 6 ring members, alkyl and cycloalkyl themselves optionally substituted with one or more halogen atoms or a hydroxyl radical; and R9 represents a hydrogen atom or an alkyl radical optionally substituted with one or more radicals, which may be identical or different, chosen from halogen atoms and the radicals hydroxyl, alkoxy, NH₂, NHalkyl, N(alkyl)₂, phenyl, heterocycloalkyl or heteroaryl, themselves optionally substituted with one or more radicals chosen from halogen atoms and the radicals hydroxyl, OCH₃, CH₃, —CH₂OH, CN, CF₃, OCF₃, NH₂, NHalk or N(alk)₂; or R8 and R9 form, with the nitrogen atom to which they are attached, a cyclic amine chosen from pyrrolyl, piperidyl, morpholinyl, pyrrolidinyl, azetidinyl and piperazinyl, optionally substituted with one or more alkyl radicals, which may be identical or different, themselves optionally substituted with one or more halogen atoms;

the NR8R9 radical may also be such that either R8 and R9, which may be identical or different, are such that R8 represents a hydrogen atom or an alkyl radical containing at most 3 carbon atoms optionally substituted with one or more halogen atoms or a hydroxyl radical; and R9 represents a hydrogen atom or an alkyl radical optionally substituted with one or more radicals, which may be identical or different, chosen from halogen atoms and the radicals hydroxyl, alkoxy, NH₂, NHalkyl, N(alkyl)₂, phenyl, morpholinyl, pyrrolidinyl, piperidinyl or pyridine, the latter rings being themselves optionally substituted with one or more radicals chosen from halogen atoms and the radicals hydroxyl, OCH₃, CH₃, CF₃, OCF₃, NH₂, NHalk or N(alk)₂; or R8 and R9 form, with the nitrogen atom to which they are attached, a cyclic amine chosen from piperidyl, morpholinyl, pyrrolidinyl and piperazinyl optionally substituted with one or more radicals chosen from halogen atoms and methyl radicals;

the NR8R9 radical may also represent the values defined above for NRaRb.

A subject of the present invention is thus the products of formula (I) as defined above in which R, R2, R3, R4, R5, Z and the ring (N) have the meanings indicated above or hereinafter and R1 and R6 are such that: either R1 represents —X1-R7 with X1 representing —CH₂— and R6 represents a hydrogen atom or the radicals hydroxyl, CH₂—OH; —CO—N(CH₃)₂, —CO—NHCH₃, —CO—NH—(CH₂)₂—N(CH₃)₂ and —CO₂Et; or R1 represents —X2-R7 with X2 representing:

—O—, —CHOH—, —CH(OH)—CH₂—, —CO—, —CHNH₂—, —NH—CH₂—, —N(CH₃)—CH₂— and CH₂—NH—CH₂—; and R6 represents hydrogen;

and R7 is chosen from the radicals pyrrolidinyl, piperidinyl, piperazinyl, pyrimidinyl, morpholinyl, thiomorpholinyl, tetrahydrofuranyl, phenyl, pyridyl, thienyl, thiazolyl, dithiazolyl, pyrazolyl, pyrazinyl, furyl, imidazolyl, pyrrolyl, oxazolyl, isoxazolyl, benzodihydrofuranyl, benzoxodiazolyl, benzothiodiazolyl, benzothienyl, quinolyl, isoquinolyl;

all these radicals represented by R7 being optionally substituted with one or more radicals, which may be identical or different, chosen from halogen atoms and the radicals hydroxyl, methyl, methoxy, hydroxymethyl, alkoxymethyl, cyano, NH₂, NHalk, N(alk)₂, —CH₂—NH₂, —CH₂—NHalk, —CH₂—N(alk)₂, phenyl, morpholinyl and

CH₂-morpholinyl, themselves optionally substituted with one or more radicals, which may be identical or different, chosen from halogen atoms and the radicals hydroxyl, CH₂, OCH₃, —CH₂OH, CN, CF₂, OCF₃, NH₂, NHalk or N(alk)₂;

said products of formula (I) being in all the possible racemic, enantiomeric and diastereoisomeric isomer forms, and also the addition salts with inorganic and organic acids of said products of formula (I).

A subject of the present invention is in particular the products of formula (I) as defined above, corresponding to the following names:
{4-[4-(4-fluorophenylamino)pyrimidin-2-ylamino]phenyl}-[4-(methyloxazol-2-ylmethylamino)piperidin-1-yl]methanone;
{4-[4-(4-fluorophenylamino)pyrimidin-2-ylamino]phenyl}-[3-(methyl-1H-pyrrole-2-ylmethylamino)piperidin-1-yl]methanone (racemic);
1-{4-[4-(4-fluorophenylamino)pyrimidin-2-ylamino]-benzoyl}-3-pyridin-3-ylmethylpiperidine-3-carboxylic acid methylamide (racemic);
N-4-(4-fluoro-3-methylphenyl)-N-2-(4-{3-[(2-methanesulphonylethylamino)methyl]pyrrolidine-1-sulphonyl}-phenyl)pyrimidine-2,4-diamine (racemic);
N-4-(4-fluoro-3-methylphenyl)-N-2-[4-(3-{[(1-methyl-1H-pyrrol-2-ylmethyl)amino]methyl}pyrrolidine-1-sulphonyl)phenyl]pyrimidine-2,4-diamine (racemic);
4-pyrrolidin-1-ylmethyl-1-{4-[4-(4-fluoro-3-methylphenylamino)pyrimidin-2-ylamino]benzenesulphonyl}-piperidin-4-ol;
{4-[4-(4-fluoro-3-methylphenylamino)pyrimidin-2-ylamino]phenyl}[4-(methylpyridin-2-ylmethylamino)piperidin-1-yl]methanone;
{4-[4-(4-fluoro-3-methylphenylamino)pyrimidin-2-ylamino]phenyl}-[4-(methylpyridin-4-ylmethylamino)piperidin-1-yl]methanone;
{4-[(1,5-dimethyl-1H-pyrazol-4-ylmethyl)methylamino]piperidin-1-yl}-{4-[4-(4-fluoro-3-methylphenylamino)pyrimidin-2-ylamino]phenyl}methanone;
{4-[(2-aminopyridin-3-ylmethyl)methylamino]piperidin-1-yl}-{4-[4-(4-fluoro-3-methylphenylamino)pyrimidin-2-ylamino]phenyl}methanone;
4-{[(1-{4-[4-(4-fluoro-3-methylphenylamino)pyrimidin-2-ylamino]benzoyl}piperidin-4-yl)methylamino]methyl}-1,5-dimethyl-1H-pyrrole-2-carbonitrile;
{4-[(2,4-dimethylthiazol-5-ylmethyl)methylamino]piperidin-1-yl}-{4-[4-(4-fluoro-3-methylphenylamino)pyrimidin-2-ylamino]phenyl}methanone;
(1-{[4-({4-[(4-fluorophenyl)amino]pyrimidin-2-yl}amino)phenyl]sulphonyl}piperidin-4-yl)(pyridin-3-yl)-methanamine;

said products of formula (I) being in all the possible racemic, enantiomeric and diastereoisomeric isomer forms, and also the addition salts with inorganic and organic acids of said products of formula (I).

A subject of the present invention is also the processes for preparing the products of formula (I) as defined above or using the methods known to those skilled in the art.

A subject of the present invention is in particular the process for preparing the products of formula (I) as defined above, characterized in that a product of formula (II):

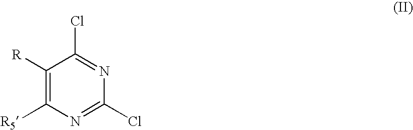

in which R5′ has the meaning indicated above for R5 in which the possible reactive functions are optionally protected,

is reacted with a product of formula (III):

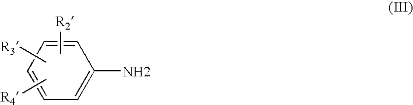

in which R2′, R3′ and R4′ have the meanings indicated above respectively for R2, R3 and R4 in which the possible reactive functions are optionally protected,

so as to obtain a product of formula (IV)

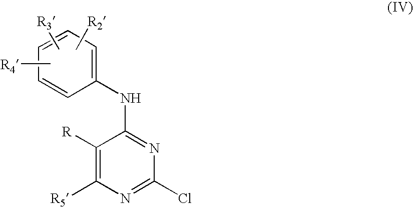

in which R2′, R3′, R4′ and R5′ have the meanings indicated above,

which product of formula (IV) is reacted with the aniline of formula (V):

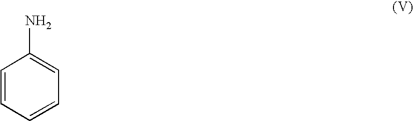

so as to obtain a product of formula (VI):

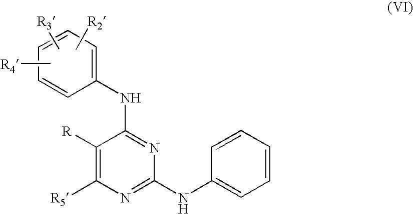

in which R2′, R3′, R4′ and R5′ have the meanings indicated above,

pathway a) z=SO₂, which product of formula (VI) is reacted with chlorosulphonic acid SO₂(OH)Cl so as to obtain the corresponding product of formula (VII):

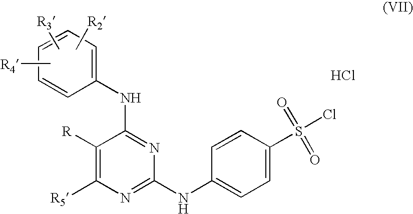

in which R2′, R3′, R4′ and R5′ have the meanings indicated above, which product of formula (VII) is reacted with an amine of formula (VIII):

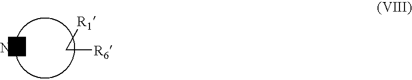

in which R1′ and R6′ have the meanings indicated above respectively for R1 and R6, in which the possible reactive functions are optionally protected with protective groups, so as to obtain a product of formula (Ia):

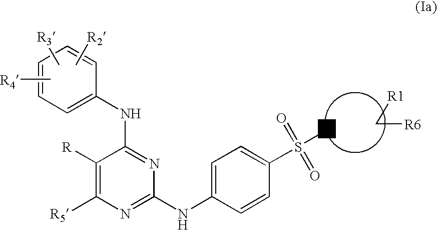

in which R1′, R2, R3, R4, R5 and R6′ have the meanings indicated above,

pathway b) z=CO which product of formula (IV) as defined above is reacted with 4-aminobenzoic acid methyl ester so as to obtain the product of formula (IX):

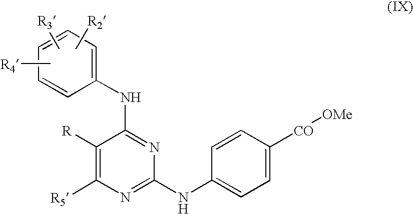

in which R2′, R3′, R4′ and R5′ have the meanings indicated above, which product of formula (IX) is saponified to its corresponding acid of formula (X):

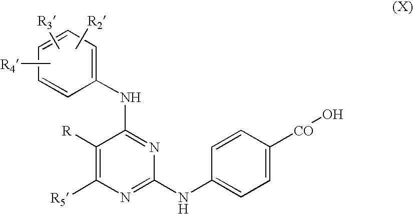

in which R2′, R3′, R4′ and R5′ have the meanings indicated above,

which product of formula (X) is reacted with an amine of formula (VIII) as defined above:

so as to obtain a product of formula (Ib):

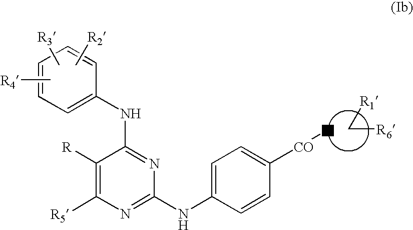

in which R2′, R3′, R4′, R5′, R1′ and R6′ have the meanings indicated above,

which products of formulae (Ia) and (Ib) may be products of formula (I) in which, respectively, z represents SO₂ and z represents CO, and which, so as to obtain products of formula (I) or other products of formula (I), can be subjected, if desired and if necessary, to one or more of the following conversion reactions in any order:

a) a reaction of oxidation of an alkylthio group to the corresponding sulphoxide or sulphone group,

b) a reaction of conversion of an alkoxy function to a hydroxyl function, or else of a hydroxyl function to an alkoxy function,

c) a reaction of oxidation of an alcohol function to an aldehyde or ketone function,

d) a reaction of removal of the protective groups that may be borne by the protected reactive functions,

e) a salification reaction with an inorganic or organic acid so as to obtain the corresponding salt,

f) a reaction to resolve the racemic forms into resolved products,

said products of formula (I) thus obtained being in all the possible racemic, enantiomeric and diastereoisomeric isomer forms.

Under preferred conditions of implementation of the invention, the processes described above can be carried out in the following way:

The product of formula (II) is subjected to the action of the product of formula (III) as defined above, in particular in an alcohol, such as, for example, butanol, propanol or ethanol, or dimethylformamide between 80 and 140° C., so as to give a product of formula (IV) as defined above.

The product of formula (IV) thus obtained is subjected to the action of the aniline of formula (V) as defined above, in particular in an alcohol such as, for example, butanol or dimethylformamide, in the presence or absence of a strong acid (HCl) in a catalytic amount under reflux conditions so as to give a product of formula (VI) as defined above.

According to pathway a) defined above, the product of formula (VI) is subjected to the action of chlorosulphonic acid, in particular first at 0° C. and then at ambient temperature, so as to give a product of formula (VII) as defined above.

The product of formula (VII) thus obtained is subjected to the action of an amine of formula (VIII) as defined above, in particular in dichloromethane or a dichloromethane/THF mixture or dimethylformamide at ambient temperature, in the presence of an organic base such as triethylamine, diisopropylethylamine or N-methylmorpholine, so as to give a product of formula (Ia) as defined above.

According to pathway b) defined above, the product of formula (IV) as defined above is subjected to the action of the 4-aminobenzoic acid methyl ester, in particular in an alcohol such as butanol at a temperature of 100 to 140° C., so as to give the product of formula (IX) as defined above.

This product of formula (IX) is saponified to its corresponding acid formula (X) by carrying out the process according to the usual methods known to those skilled in the art, such as, in particular, by the action of sodium hydroxide or of potassium hydroxide in water.

The product of formula (X) thus obtained is reacted with the amine of formula (VIII) defined above according to the coupling methods known to those skilled in the art, such as, for example, by peptide coupling in the presence of a coupling agent such as BOP, DCC or TBTU in a solvent such as, for example, dimethylformamide or dichloromethane, so as to give a product of formula (Ib) as defined above.

According to the values of R1′, R2′, R3′, R4′, R5′ and R6′, the products of formulae (Ia) and (Ib) as defined above can therefore constitute products of formula (I) as defined above, in which, respectively, z represents SO₂ and z represents CO, or can be converted to products of formula (I) by the usual methods known to those skilled in the art, and for example by being subjected to one or more of the reactions a) to f) indicated above.

Moreover, it may be noted that such reactions a) to f) for converting substituents to other substituents may also be carried out on the starting products and also on the intermediates as defined above before continuing the synthesis according to the reactions indicated in the processes above.

The various reactive functions which certain compounds of the reactions defined above may carry may, if necessary, be protected: these are for example hydroxyl, acyl or alternatively amino and monoalkylamino radicals which may be protected with appropriate protective groups.

The following non-exhaustive list of examples of protection of reactive functions may be mentioned:
the hydroxyl groups may be protected for example with alkyl radicals such as tert-butyl, trimethylsilyl, tert-butyldimethylsilyl, methoxymethyl, tetrahydropyranyl, benzyl or acetyl,
the amino groups may be protected for example with acetyl, trityl, benzyl, tert-butoxycarbonyl, benzyloxycarbonyl and phthalimido radicals or other radicals known in peptide chemistry: the amine functions may in particular be protected with a group such as Boc or CH₂-phenyl and may then be released under the customary conditions known to a person skilled in the art.

The reactions to which the products of formula (I′) as defined above may be subjected, if desired or if necessary, may be carried out, for example, as indicated below.

The saponification reactions may be carried out according to the usual method known to a person skilled in the art, such as for example in a solvent such as methanol or ethanol, dioxane or dimethoxyethane, in the presence of sodium hydroxide or potassium hydroxide.

The reduction or oxidation reactions may be carried out according to the usual methods known to a person skilled in the art such as for example in a solvent such as ethyl ether or tetrahydrofuran, in the presence of sodium borohydride or lithium aluminum hydride, or for example in a solvent such as acetone or tetrahydrofuran in the presence of potassium permanganate or pyridinium chlorochromate.

a) The optional alkylthio groups of the products described above may be, if desired, converted to the corresponding sulphoxide or sulphone functions under the customary conditions known to a person skilled in the art such as for example with peracids such as for example peracetic acid or meta-chloroperbenzoic acid or alternatively with oxone, sodium periodate in a solvent such as for example methylene chloride or dioxane at room temperature.

The production of the sulphoxide function may be promoted by an equimolar mixture of the product containing an alkylthio group and the reagent such as in particular a peracid.

The production of the sulphone function may be promoted by a mixture of the product containing an alkylthio group with an excess reagent such as in particular a peracid.

b) The optional alkoxy functions such as in particular methoxy of the products described above may be, if desired, converted to a hydroxyl function under the customary conditions known to a person skilled in the art, for example with boron tribromide in a solvent such as for example methylene chloride, with pyridine hydrobromide or hydrochloride or alternatively with hydrobromic or hydrochloric acid in water or trifluoroacetic acid under reflux.

c) The optional alcohol functions of the products described above may be, if desired, converted to an aldehyde or ketone function by oxidation under the customary conditions known to a person skilled in the art such as, for example by the action of manganese oxide in order to obtain the aldehydes or by the action of potassium permanganate or pyridinium chlorochromate in order to obtain the ketones.

d) The removal of the protective groups such as for example those indicated above may be carried out under the customary conditions known to a person skilled in the art, in particular by acid hydrolysis carried out with an acid such as hydrochloric, benzenesulphonic or para-toluenesulphonic, formic or trifluoroacetic acid or alternatively by catalytic hydrogenation.

The phthalimido group may in particular be removed with hydrazine.

A list of the various protective groups which can be used may be found for example in patent BF 2 499 995.

e) The products described above may, if desired, be subjected to salification reactions for example with an inorganic or organic acid according to the usual method known to a person skilled in the art.

f) The optional optically active forms of the products described above may be prepared by resolution of the racemates according to the usual method known to a person skilled in the art.

Illustrations of such reactions defined above are given in the preparation of the examples described below.

The starting materials of formulae (II), (III), (V) and (VIII) may be known, may be commercially obtained or may be prepared according to the usual method known to a person skilled in the art, in particular from commercial products, for example by subjecting them to one or more reactions known to a person skilled in the art such as for example the reactions described above in a) to f).

The products of formula (II) which are therefore pyrimidine derivatives and the products of formula (III) which are aniline derivatives may be commercial products such as for example dichloropyrimidine, trichloropyrimidine, 4-fluoroaniline, 3,4-difluoroaniline, 4-fluoro-3-chloroaniline, or aniline.

The anilines of formula (III) may be in particular commercial anilines such as for example the following trihalogenated anilines:
3,4,5-trifluoroaniline,
2,3,4-trifluoroaniline,
2-chloro-4,6-difluoroaniline,
2,4,5,-trifluoroaniline,
3-chloro-2,4-difluoroaniline,
2,4-dichloro-5-fluoroaniline, and
4-trifluoromethylphenylamine.

The aniline of formula (V) is commercially available.

The amines of formula (VIII) may in particular be commercially available amines such as, for example, the following trihalogenated anilines:
ethyl-3-(pyridin-2-ylmethyl)piperidine 3-carboxylate dihydrochloride,
ethyl-3-(pyridin-3-ylmethyl)piperidine 3-carboxylate dihydrochloride,
ethyl-3-(pyridin-4-ylmethyl)piperidine 3-carboxylate dihydrochloride,
4-benzyl-4-hydroxypiperidine,
2-(piperidin-4-yloxy)pyrazine dihydrochloride,
4-(piperidin-4-yloxy)pyridine dihydrochloride,
2-(piperidin-4-yloxy)pyrimidine dihydrochloride,
4-phenoxypiperidine hydrochloride,
2-(piperidin-4-yloxy)pyridine dihydrochloride,
2-piperidin-4-ylmethylpyridine dihydrochloride,
4-piperidin-4-ylmethylpyridine dihydrochloride,
3-piperidin-4-ylmethylpyridine dihydrochloride,
(R)-(4-fluorophenyl)-1-piperidine-4-methanamine,
(S)-(4-fluorophenyl)-1-piperidine-4-methanamine,
(R)-phenyl-1-piperidine-4-methanamine,
(S)-phenyl-1-piperidine-4-methanamine, and
(4-fluorophenyl)piperidin-4-ylmethanol.

The preparations of the amines of formula (VIII) that are not commercially available can be carried out according to methods known to those skilled in the art.

It may be indicated that, in order to obtain products of formula (I) as defined above in which the ring (N) contains a carbon-based bridge comprising from 1 to 3 carbons, use may be made, as starting products, of the bicyclic amines that can be obtained from commercially available compounds such as tropinone, or pseudo-pelletierine according to the references below:
Tetrahedron 2002, 58, 5669-5674
J. Org. Chem. 1996, 61, 3849-3862
J. Med. Chem. 1993, 36, 3703-3720
J. Chem. Soc. Perkin Transl 1991, 1375-1381
J. Med. Chem. 1994, 37, 2831-2840

By way of examples of ring (N), mention may be made of the following compounds:
9-azabicyclo[3.3.1]nonan-3-amine

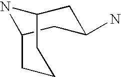

6-azabicyclo[3.2.1]octan-3-amine

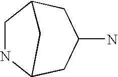

3-azabicyclo[3.2.1]octan-8-amine

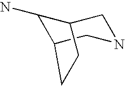

3-azabicyclo[3.3.1]nonan-9-amine

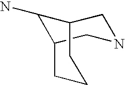

These bicycles which constitute examples of a ring (N) being substituted with R1 and R6 as defined above and optionally protected, if necessary, and these bicycles being linked to z via their intracyclic nitrogen.

Examples of aldehydes or of ketones of formula (X) are given in the experimental section by way of non-limiting examples.

The experimental section hereinafter gives non-limiting examples of preparation of products of formula (I) according to the present invention and also examples of non-limiting starting products used in these preparations.

Finally, the subject of the present invention is, as novel industrial products, compounds of formulae (VII), (IX) and (X).

The products of formula (I) as defined above and also their addition salts with acids have advantageous pharmacological properties.

The compounds of the present invention may therefore inhibit the activity of kinases, in particular IKK1 and IKK2 with an IC50 of less than 10 μM.

The compounds of the present invention may thus inhibit the activation of NF-KB, and the production of cytokines with IC50 values of less than 10 μM.

The compounds of the present invention may thus inhibit the proliferation of a large panel of tumor cells with IC50 values of less than 10 μM.

The compounds of formula (I) may therefore have a medicament activity, in particular as IKK1 and IKK2 inhibitors and may be used in the prevention or treatment of diseases in which the inhibition of IKK1 or IKK2 is beneficial. For example, the prevention or treatment of diseases such as inflammatory diseases or diseases with an inflammatory component such as for example inflammatory arthritis including rheumatoid arthritis, spondylitic osteoarthritis, Reiter's syndrome, psoriatic arthritis, bone resorption diseases; multiple sclerosis, inflammatory bowel diseases including Crohn's disease; asthma, chronic pulmonary obstruction, emphysema, rhinitis, acquired myasthenia, Graves' disease, graft rejection, psoriasis, dermatitis, allergic disorders, immune system diseases, cachexia, severe acute respiratory syndrome, septic shock, cardiac insufficiency, myocardial infarction, atherosclerosis, reperfusion lesions, AIDS, cancer and disorders characterized by insulin resistance such as diabetes, hyperglycemia, hyperinsulinemia, dyslipidemia, obesity, polycystic ovary diseases, hypertension, cardiovascular disorders, syndrome X, autoimmune diseases such as in particular systemic lupus, lupus erythematosus, glomerulonephritis induced by immune system deficiencies, insulin-dependent autoimmune diabetes, retinitis pigmentosa, aspirin-sensitive rhinosinusitis.

The products of formula (I) according to the present invention as modulators of apoptosis may be useful in the treatment of various human diseases including aberrations in apoptosis such as cancer: such as in particular, but without limitation, follicular lymphomas, carcinomas with p53 mutations, hormone-dependent breast, prostate and ovarian tumors, and precancerous lesions such as familial adenomatous polyposis, viral infections (such as in particular, but without limitation, those caused by the Herpes virus, the poxvirus, the Epstein-Barr virus, Sindbis virus and adenovirus), myelodysplastic syndromes, ischemic disorders associated with myocardial infarction, cerebral congestion, arrhythmia, atherosclerosis, hepatic disorders induced by toxins or alcohol, hematological disorders such as in particular, but without limitation, chronic anemia and aplastic anemia, degenerative diseases of the musculoskeletal system such as in particular, but without limitation, osteoporosis, cystic fibrosis, kidney diseases and cancer.

It is therefore evident that the compounds according to the invention have anticancer activity and activity in the treatment of other proliferative diseases such as psoriasis, restenosis, atherosclerosis, AIDS for example, and in diseases caused by the proliferation of the vascular smooth muscle cells, angiogenesis and in rheumatoid arthritis, neurofibromatosis, atherosclerosis, pulmonary fibrosis, restenosis following angioplasty or vascular surgery, the formation of hypertrophic scars, angiogenesis and endotoxic shock.

These medicaments find their use in therapy, in particular in the treatment or prevention of diseases caused or exacerbated by the proliferation of cells and in particular of tumor cells.

As inhibitor of tumor cell proliferation, these compounds are useful in the prevention and treatment of leukemia, both primary and metastatic solid tumors, carcinomas and cancers, in particular: breast cancer, lung cancer, cancer of the small intestine, cancer of the colon and rectum, cancer of the respiratory tracts, of the oropharynx and of the hypopharynx, cancer of the esophagus, liver cancer, stomach cancer, cancer of the bile ducts, cancer bile vesicle, pancreatic cancer, cancer of the urinary tracts including kidney, urothelium and bladder, cancer of the female genital tract including uterine, cervical and ovarian cancers, chloriocarcinoma and trophoblastoma; cancers of the male genital tract including cancer of the prostate, the seminal vesicles and the testicles, tumors of the germ cells; cancers of the endocrine glands including cancer of the thyroid, the pituitary gland and the adrenal glands; skin cancer including hemangiomas, melanomas, sarcomas, including Kaposi's sarcoma; tumors of the brain, the nerves, the eyes and the meninges, including astrocytomas, gliomas, glioblastomas, retinoblastomas, neurinomas, neuroblastomas, schwannomas, meningiomas; hematopoietic malignant tumors; leukemias such as acute lymphoid leukemia, acute myeloid leukemia, chronic myeloid leukemia, chronic lymphoid leukemia, chloromas, plasmocytomas, T or B cell leukemia, Hodgkin or non-Hodgkin lymphomas, myelomas, various malignant hemopathies.

The subject of the present invention is in particular the combinations defined as follows.

According to the present invention, the compound(s) of formula (I) may be administered in combination with one or more anticancer active ingredients, in particular antitumor compounds such as alkylating agents such as alkyl sulphonates (busulphan), dacarbazine, procarbazine, nitrogen mustards (chlormethine, melphalan, chlorambucil), cyclophosphamide, ifosfamide; nitrosoureas such as carmustine, lomustine, semustine, streptozocin; antineoplastic alkaloids such as vincristine, vinblastine; taxanes such as paclitaxel or taxotere; antineoplastic antibiotics such as actinomycin; intercalating agents, antineoplastic antimetabolites, antagonists of folates, methotrexate; inhibitors of the synthesis of purines; purine analogues such as mercaptopurine, 6-thioguanine; inhibitors of the synthesis of pyrimidines, aromatase inhibitors, capecitabine, pyrimidine analogues such as fluorouracil, gemcitabine, cytarabine and cytosine arabinoside; brequinar; inhibitors of topoisomerases such as camptothecin or etoposide; anticancer hormonal agonists and antagonists including tamoxifen; kinase inhibitors, imatinib; growth factor inhibitors; anti-inflammatory agents such as pentosan polysulphate, corticosteroids, prednisone, dexamethasone; antitopoisomerases such as etoposide, anthracyclines including doxorubicin, bleomycin, mitomycin and methramycin; anticancer metal complexes, platinum complexes, cisplatin, carboplatin, oxaliplatin; interferon-alpha, triphenylthiophosphoramide, altretamine; antiangiogenic agents; thalidomide; immunotherapy adjuvants; vaccines.

According to the present invention, the compounds of formula (I) may also be administered in combination with one or more other active ingredients useful in one of the pathologies indicated above, for example an antiemetic, antipain, anti-inflammatory and anticachexia agent.

The subject of the present invention is thus, as medicaments, the products of formula (I) as defined above and also the addition salts with pharmaceutically acceptable inorganic and organic acids of said products of formula (I).

The subject of the present invention is in particular, as medicaments, the products of formula (I) as defined above corresponding to the following names:
{4-[4-(4-fluorophenylamino)pyrimidin-2-ylamino]phenyl}-[4-(methyloxazol-2-ylmethylamino)piperidin-1-yl]methanone;
{4-[4-(4-fluorophenylamino)pyrimidin-2-ylamino]phenyl}-[3-(methyl-1H-pyrrole-2-ylmethylamino)piperidin-1-yl]methanone (racemic);
1-{4-[4-(4-fluorophenylamino)pyrimidin-2-ylamino]-benzoyl}-3-pyridin-3-ylmethylpiperidine-3-carboxylic acid methylamide (racemic);
N*4*-(4-fluoro-3-methyl phenyl)-N*2*-(4-{3-[(2-methanesulphonylethylamino)methyl]pyrrolidine-1-sulphonyl}phenyl)pyrimidine-2,4-diamine (racemic);
N*4*-(4-fluoro-3-methylphenyl)-N*2*-[4-(3-{[(1-methyl-1H-pyrrol-2-ylmethyl)amino]methyl}pyrrolidine-1-sulphonyl)phenyl]pyrimidine-2,4-diamine (racemic);
4-pyrrolidin-1-ylmethyl-1-{4-[4-(4-fluoro-3-methylphenylamino)pyrimidin-2-ylamino]benzenesulphonyl}-piperidin-4-ol;
{4-[4-(4-fluoro-3-methylphenylamino)pyrimidin-2-ylamino]phenyl}[4-(methylpyridin-2-ylmethylamino)piperidin-1-yl]methanone;
{4-[4-(4-fluoro-3-methylphenylamino)pyrimidin-2-ylamino]phenyl}-[4-(methylpyridin-4-ylmethylamino)piperidin-1-yl]methanone;
{4-[(1,5-dimethyl-1H-pyrazol-4-ylmethyl)methylamino]piperidin-1-yl}-{4-[4-(4-fluoro-3-methylphenylamino)pyrimidin-2-ylamino]phenyl}methanone;
{4-[(2-aminopyridin-3-ylmethyl)methylamino]piperidin-1-yl}-{4-[4-(4-fluoro-3-methylphenylamino)pyrimidin-2-ylamino]phenyl}methanone;
4-{[(1-{4-[4-(4-fluoro-3-methylphenylamino)pyrimidin-2-ylamino]benzoyl}piperidin-4-yl)methylamino]methyl}-1,5-dimethyl-1H-pyrrole-2-carbonitrile;
{4-[(2,4-dimethylthiazol-5-ylmethyl)methylamino]piperidin-1-yl}-{4-[4-(4-fluoro-3-methylphenylamino)pyrimidin-2-ylamino]phenyl}methanone
(1-{[4-({4-[(4-fluorophenyl)amino]pyrimidin-2-yl}amino)phenyl]sulphonyl}piperidin-4-yl)(pyridin-3-yl)-methanamine;

and also the addition salts with pharmaceutically acceptable inorganic and organic acids of said products of formula (I).

The subject of the present invention is also the pharmaceutical compositions containing, as active ingredient, at least one of the products of formula (I) as defined above or a pharmaceutically acceptable salt of this product or a prodrug of this product and a pharmaceutically acceptable carrier.

The subject of the present invention is also pharmaceutical compositions containing, as active ingredient, at least one of the products of formula (I), the names of which are given above, or a pharmaceutically acceptable salt of this product or a prodrug of this product and a pharmaceutically acceptable carrier.

The subject of the present invention is particularly the use of the products of formula (I) as defined above or of pharmaceutically acceptable salts of these products for the preparation of a medicament intended for the treatment or prevention of a disease by inhibiting the activity of the protein kinase IKK.

The subject of the present invention is thus the use, as defined above, in which the protein kinase is in a mammal.

The subject of the present invention is thus the use of a product of formula (I) as defined above for the preparation of a medicament intended for the treatment or prevention of a disease chosen from the diseases indicated above.

The subject of the present invention is in particular the use of a product of formula (I) as defined above for the preparation of a medicament intended for the treatment or prevention of a disease chosen from the following group: inflammatory diseases, diabetes and cancers.

The subject of the present invention is in particular the use of a product of formula (I) as defined above for the preparation of a medicament intended for the treatment or prevention of inflammatory diseases.

The subject of the present invention is in particular the use of a product of formula (I) as defined above for the preparation of a medicament intended for the treatment or prevention of diabetes.

The subject of the present invention is in particular the use of a product of formula (I) as defined above for the preparation of a medicament intended for the treatment of cancers.

The subject of the present invention is in particular the use of a product of formula (I) as defined above intended for the treatment of solid or liquid tumors.

The subject of the present invention is in particular the use of a product of formula (I) as defined above intended for the treatment of cancers resistant to cytotoxic agents.

The subject of the present invention is in particular the use of a product of formula (I) as defined above for the preparation of medicaments intended for cancer chemotherapy.

The subject of the present invention is in particular the use of a product of formula (I) as defined above for the preparation of medicaments intended for cancer chemotherapy alone or in combination or in the form of a combination as defined above.

The subject of the present invention is in particular the use of a product of formula (I) as defined above as IKK inhibitors.

The present invention relates most particularly to the products of formula (I) as defined above which constitute Examples 1 to 260 of the present invention.

The following examples illustrate the invention without however limiting it.

### EXPERIMENTAL SECTION

Procedure 1: Preparation of sulphonyl chloride hydrochlorides

Procedure 1a: 4-[4-(4-Fluorophenylamino)pyrimidin-2-ylamino]-benzenesulphonyl chloride hydrochloride

Stage 1: (2-Chloropyrimidin-4-yl)-(4-fluorophenyl)amine

10 ml of 4-fluoroaniline and then 18 ml of diisopropyl-ethylamine are added to a mixture containing 15 g of dichloropyrimidine in 200 ml of n-butanol, with stirring. The reaction mixture is brought to reflux with stirring, for 2 hours. The reaction medium is cooled, and concentrated to dryness. A solution of K₂CO₃ is added to the residue and the mixture is extracted 3 times with ethyl acetate, washing is carried out with a saturated solution of NaCl and the product is dried over Na₂SO₄. The reaction crude is purified by silica column chromatography (DCM then 30% of ethyl acetate in DCM). During concentration, 11 g of expected product crystallize (MH+=224), Mp=172-174° C.

Stage 2: N-4-(4-Fluorophenyl)-N-2-phenylpyrimidine-2,4-diamine

10.5 g of (2-chloropyrimidin-4-yl)-(4-fluorophenyl)amine in solution in 300 ml of n-butanol are brought to 140° C. at reflux in the presence of 4.3 ml of aniline overnight. The reaction medium is cooled. The suspension obtained is filtered. The crystals are taken up in ethyl acetate and washed with a 10% solution of K₂CO₃ and then with a saturated solution of NaCl. After drying over Na₂SO₄, the organic phase is concentrated under vacuum. The reaction crude is purified by silica column chromatography (THF/MeOH/DCM, Oct. 5, 1985). The expected N-4-(4-fluorophenyl)-N-2-phenylpyrimidine-2,4-diamine crystallizes during concentration and 10.5 g of the product are obtained by filtration. MH+=281, Mp=161° C.

Stage 3: 4-[4-(4-Fluorophenylamino)pyrimidin-2-ylamino]-benzenesulphonyl chloride hydrochloride

7.5 g of N-4-(4-fluorophenyl)-N-2-phenylpyrimidine-2,4-diamine are added, in small portions, while maintaining the temperature at around 0° C., to a three-necked round-bottomed flask under a stream of nitrogen, containing chlorosulphonic acid. The reaction medium is left at ambient temperature for 18 h. The mixture is poured dropwise (with care) onto ice. The precipitate obtained is filtered off and washed with distilled water. After dissolution of the solids in 1 l of ethyl acetate, drying over Na₂SO₄ and concentration under vacuum, a whitish oil is obtained. This oil precipitates after dispersion in 200 ml of ether. 10.5 g of 4-[4-(4-fluorophenylamino)pyrimidin-2-ylamino]benzenesulphonyl chloride hydrochloride are obtained by filtration of the ethereal suspension. MH+=360, Mp poorly defined.

Procedure 1b: 4-[4-(3,4-Difluorophenylamino)pyrimidin-2-ylamino]benzenesulphonyl chloride hydrochloride

Stage 1: 4-Chloro-N-(3,4-difluorophenyl)pyrimidin-2-amine

The preparation of this compound is carried out according to the same process as for procedure 1a, based on the reaction of 9.21 g of dichloropyrimidine with 8 g of 3,4-difluoroaniline: 10.3 g of expected product are thus obtained.

Stage 2: N4-(3,4-Difluorophenyl)-N-2-phenylpyrimidine-2,4-diamine

The preparation of this compound is carried out according to the same process as for Example 1, based on the reaction of 7 g of (2-chloropyrimidin-4-yl)-(3,4-difluorophenyl)amine obtained in stage 1 above with 2.72 g of aniline: 8 g of expected product are thus obtained.

Stage 3: 4-[4-(3,4-Difluorophenylamino)pyrimidin-2-ylamino]benzenesulphonyl chloride hydrochloride

The preparation of this compound is carried out according to the same process as for Example 1, based on the reaction of 8 g of N-4-(3,4-difluorophenyl)-N-2-phenylpyrimidine-2,4-diamine obtained in the stage above with chlorosulphonic acid: 9 g of expected product are thus obtained.

Procedure 1c: 4-[4-(4-Fluoro-3-methylphenylamino)pyrimidin-2-ylamino]benzenesulphonyl chloride hydrochloride

Stage 1: (2-Chloropyrimidin-4-yl)-(4-fluoro-3-methyl-phenyl)amine

The preparation of this compound is carried out according to the same process as for Example 1, based on the reaction of 5.3 g of 4-fluoro-3-methylphenylamine with 6.3 g of 2,4-dichloropyrimidine: 3.8 g of expected product are obtained (melting point=130-131° C.) (trituration in isopropyl ether).

Stage 2: N-4-(4-Fluoro-3-methylphenyl)-N-2-phenyl-pyrimidine-2,4-diamine

The preparation of this compound is carried out according to the same process as for Example 1, based on the reaction of 2.8 g of (2-chloropyrimidin-4-yl)-(4-fluoro-3-methylphenyl)amine obtained above and 1.2 ml of aniline: 2.2 g of expected product are obtained (melting point=134-135° C.) (trituration in isopropyl ether).

Stage 3: 4-[4-(4-Fluoro-3-methylphenylamino)pyrimidin-2-ylamino]benzenesulphonyl chloride hydrochloride

The preparation of this compound is carried out according to the same process as for Example 1, based on the reaction of 2 g of N-4-(4-fluoro-3-methylphenyl)-N-2-phenylpyrimidine-2,4-diamine obtained above with chlorosulphonic acid: 1.5 g of expected product are obtained.

Procedure 1d: 4-[5-Fluoro-4-(4-fluoro-3-phenylamino)pyrimidin-2-ylamino]benzenesulphonyl hydrochloride

Step 1: (2-Chloro-5-fluoropyrimidin-4-yl)-(4-fluoro-3-methylphenyl)amine

4 g of 2,4-dichloro-5-fluoropyrimidine, 2.67 g of 4-fluoroaniline and 4 ml of DIPEA in solution in 75 ml of n-butanol. The reaction medium is brought to 80° C. for 1 h 30. The reaction medium is concentrated to dryness, taken up with an H₂O/K₂CO₃ solution and then extracted with EtOAc. After washing with H₂O/NaCl and drying over Na₂SO₄, the reaction crude is purified on an SiO₂ column and eluted with DCM/MeOH (V/V, 99/1). 5 g of expected product are obtained.

Step 2: (5-Fluoro)-N*4*-(4-fluorophenyl)-N*2*-phenyl-pyrimidine-2,4-diamine

3 g of product obtained in step 1 are dissolved in 30 ml of n-butanol containing 1 g of aniline. The reaction mixture is heated at 150° C. for 3 h. The hydrochloride crystallizes under hot conditions. The product is left to cool, after filtration, the solid obtained is washed with ether. 3.4 g of expected product are obtained.

Step 3: 4-[5-Fluoro-4-(4-fluoro-3-phenylamino)pyrimidin-2-ylamino]benzenesulphonyl hydrochloride

3.4 g of (5-Fluoro)-N*4*-(4-fluorophenyl)-N*2*-phenyl-pyrimidine-2,4-diamine are added, in small portions, while maintaining the temperature at around 0° C., to a three-necked flask, under N₂, containing chlorosulphonic acid at 0° C. The reaction medium is left at ambient temperature for 18 h. The mixture is poured dropwise onto ice. The precipitate obtained is filtered off and washed with distilled water. After dissolution of the solid in 1 l of ethyl acetate, drying over Na₂SO₄ and concentration under vacuum, a whitish solid is obtained. (Yield=3.4 g).

Procedure 1e: 4-[5-Fluoro-4-(4-fluoro-3-methylphenyl-amino)pyrimidin-2-ylamino]benzenesulphonyl chloride hydrochloride

The preparation of this compound is carried out according to the same processes as in procedure 1b, replacing the 4-fluorophenylamine with 4-fluoro-3-methylphenylamine. Thus, from 4.6 g of 4-fluoro-3-methylphenylamine and 6 g of 2,4-dichloro-5-fluoropyrimidine, 11 g of expected hydrochloride are obtained.

Procedure 2: Preparation of pyrimidine-2-(4-aminobenzoic acid) derivatives

Procedure 2a: 4-[4-(4-Fluorophenylamino)pyrimidin-2-ylamino]benzoic acid

Stage 1: 4-[4-(4-Fluorophenylamino)pyrimidin-2-ylamino]benzoic acid methyl ester

A mixture containing 16 g of chloropyrimidine obtained in stage 1 of procedure 1a and 10.8 g of methyl 4-aminobenzoate in n-butanol is heated at 140° C. overnight. After cooling, the precipitate is filtered off. This precipitate is washed with Et₂O and is recrystallized from a DCM-MeOH-iPr₂O mixture. 23.5 g of expected product are thus obtained.

Stage 2: 4-[4-(4-Fluorophenylamino)pyrimidin-2-ylamino]-benzoic acid

15 g of product obtained in stage 1, in the presence of 4.5 g of sodium hydroxide in a mixture of MeOH (100 ml), water (100 ml) and dioxane (400 ml), are brought to a temperature of 40° C. overnight. The reaction medium is concentrated to dryness and taken up in 100 ml of water. The impurities are extracted with two volumes of Et₂O, and then the aqueous phase is acidified to pH 6 with 1N HCl. The precipitate formed is filtered off, rinsed with distilled water and suspended in DCM, and the solvent is evaporated off. 15 g of expected acid are obtained.

Procedure 2b: 4-[4-(4-Fluoro-3-methylphenylamino)pyrimidin-2-ylamino]benzoic acid

Stage 1: 4-[4-(4-Fluoro-3-methylphenylamino)pyrimidin-2-ylamino]benzoic acid methyl ester

A mixture containing 8 g of chloropyrimidine obtained in stage 1 of procedure 1c and 5.1 g of methyl 4-aminobenzoate is heated at 140° C. overnight. After cooling, the precipitate is filtered off. This precipitate is washed with Et₂O and is recrystallized from a DCM-MeOH-iPr₂O mixture. 10.5 g of expected product are thus obtained.

Stage 2: 4-[4-(4-Fluoro-3-methylphenylamino)pyrimidin-2-ylamino]benzoic acid

2.08 g of product obtained in stage 1, in the presence of 410 mg of sodium hydroxide in a mixture of MeOH (5 ml), water (5 ml) and dioxane (20 ml), are brought to a temperature of 40° C. overnight. The reaction medium is concentrated to dryness and taken up in 100 ml of water. The impurities are extracted with two volumes of Et₂O, and then the aqueous phase is acidified to pH 6 with 1N HCl. The precipitate formed is filtered off, rinsed with distilled water and suspended in DCM, and the solvent is evaporated off. 1.3 g of expected product are obtained.

Procedure 2c: 4-[4-(4-Trifluoromethylphenylamino)pyrimidin-2-ylamino]benzoic acid

Stage 1: (2-Chloropyrimidin-4-yl)-(4-trifluoromethyl-phenyl)amine

In the same manner as in Example 1 of procedure 2b, using 15 g of dichloropyrimidine in 200 ml of n-butanol, with stirring, 16 g of 4-trifluoromethylphenylamine are added, followed by 18 ml of diisopropylethylamine. The reaction mixture is brought to reflux overnight with stirring. The reaction medium is cooled and concentrated to dryness. A solution of K₂CO₃ is added to the residue and the mixture is extracted 3 times with ethyl acetate, washing is performed with a saturated solution of NaCl and the product is dried over Na₂SO₄. The reaction crude is purified by silica chromatography (DCM then 2% of MeOH in DCM). 5 g of expected product are obtained.

Stage 2: 4-[4-(4-Trifluoromethylphenylamino)pyrimidin-2-ylamino]benzoic acid methyl ester

In the same way as in stage 2 of procedure 1, using 4.6 g of a mixture containing 8 g of chloropyrimidine obtained in stage 1 and 2.6 g of methyl 4-aminobenzoate, 6.4 g of expected product are thus obtained.

Stage 3: 4-[4-(4-Trifluoromethylphenylamino)pyrimidin-2-ylamino]benzoic acid.

In the same way as in stage 3 of procedure 1, using 6.4 g of a mixture containing 8 g of ester obtained in stage 2 and 2.26 g of sodium hydroxide, 4.2 g of expected product are thus obtained.

Procedure 3: Preparation of the Reaction Intermediates of Sulphonamide Type

Procedure 3a: N-4-(4-Fluoro-3-methylphenyl)-N-2-[4-(4-methylaminopiperidine-1-sulphonyl)phenyl]pyrimidine-2,4-diamine

Stage 1: (1-{4-[4-(4-Fluoro-3-methylphenylamino)pyrimidin-2-ylamino]benzenesulphonyl}piperidin-4-yl)-methylcarbamic acid tert-butyl ester

3 g of sulphonyl chloride hydrochloride obtained in procedure 1c are treated with 1.7 g of 4-N-Boc-methylpiperidine in 50 ml of DCM in the presence of 2.3 ml of DIPEA overnight at ambient temperature. After usual treatment, chromatography is carried out on an SiO₂ column, elution being carried out with DCM/MeOH (97/3; v/v). 2.75 g of expected product are obtained.

Stage 2: N*4*-(4-Fluoro-3-methylphenyl)-N*2*-[4-(4-methylaminopiperidine-1-sulphonyl)phenyl]pyrimidine-2,4-diamine

The compound obtained in stage 1 is dissolved in MeOH and then treated with 35 ml of Et₂O/2N HCl overnight. The hydrochloride is filtered off and redissolved in water, the solution is basified with solid K₂CO₃ and extracted with EtOAc. After washing the organic phase with water and drying over Na₂SO₄, 2.25 g of a powder are obtained by evaporating off the solvent.

MH+=471.3

Mp=148-150° C.

NMR (1H, DMSO)

1.21-1.36 (unresolved peak, 2); 1.61 (m, 1); 1.71-1.85 (unresolved peak, 2); 2.16 (s, 3); 2.24 (d, 3); 2.27 (m, 1); 2.45 (m, 2); 3.36 (m, 2); 6.29 (d, 1); 7.12 (t, 1); 7.47 (m, 1); 7.58 (d, 2); 7.59 (m, 1); 7.97 (d, 2); 8.08 (d, 1); 9.43 (s, 1); 9.72 (s, 1).

Procedure 3b: N-2-[4-(3-Aminomethylpiperidine-1-sulphonyl)phenyl]-N-4-(4-fluorophenyl)pyrimidine-2,4-diamine (racemic)

Stage 1: 1-{4-[4-(4-Fluorophenylamino)pyrimidin-2-ylamino]benzenesulphonyl}piperidin-3-ylmethyl)carbamic acid tert-butyl ester (racemic)

According to the protocol described in stage 1 of procedure 3a, using 4 g of sulphonyl chloride hydrochloride obtained in procedure 1a and 3.43 g of the commercially available racemic amine piperidin-3-ylmethylcarbamic acid tert-butyl ester, 2.7 g of expected product are obtained.

MH+=557.1

Melting point=110° C.

Stage 2: N-2-[4-(3-Aminomethylpiperidine-1-sulphonyl)phenyl]-N-4-(4-fluorophenyl)pyrimidine-2,4-diamine (racemic)

According to the decarboxylation reaction described in stage 2 of procedure 3a, using 2.7 g of product obtained in stage 1, 2.3 g of expected product are obtained.

MH+=457.1

Melting point=207° C.

1H NMR (DMSO):

0.69 (m, 1); 1.13-1.92 (unresolved peak, 7); 2.10 (t, 1); 2.19-2.43 (2m, 2); 3.38-3.68 (2d, 2); 6.25 (d, 1); 7.13 (t, 2); 7.54 (d, 2); 7.66 (m, 2); 7.93 (d, 2); 8.10 (d, 1); 9.47 (s, 1); 9.67 (s, 1).

Procedure 3c: N-2-[4-(3-S-Aminopyrrolidine-1-sulphonyl)phenyl]-N-4-(4-fluorophenyl)pyrimidine-2,4-diamine

Stage 1: (1-{4-[4-(4-Fluorophenylamino)pyrimidin-2-ylamino]benzenesulphonyl}pyrrolidin-3-5-yl)carbamic acid tert-butyl ester

According to the protocol described in stage 1 of procedure 3a, using 400 mg of sulphonyl chloride hydrochloride obtained in procedure 1a and 198 mg of the commercially available amine pyrrolidin-3-5-ylcarbamic acid tert-butyl ester, 341 mg of expected product are obtained.

MH+=529.2

Melting point=178.2° C.

Stage 2: N-2-[4-(3-S-Aminopyrrolidine-1-sulphonyl)-phenyl]-N-4-(4-fluorophenyl)pyrimidine-2,4-diamine

According to a decarboxylation reaction using 200 mg of the compound obtained in stage 1 in 2.4 ml of a DCM-TFA mixture (v/v, 1/1), 163 mg of expected product are obtained in the form of a TFA salt.

MH+=429.0

Melting point=250° C.

Procedure 3d: N-2-[4-(3-R-Aminopyrrolidine-1-sulphonyl)phenyl]-N-4-(4-fluorophenyl)pyrimidine-2,4-diamine

Stage 1: (1-{4-[4-(4-Fluorophenylamino)pyrimidin-2-ylamino]benzenesulphonyl}pyrrolidin-3-R-yl)carbamic acid tert-butyl ester

According to the procedure described in stage 1 of procedure 3a, using 400 mg of sulphonyl chloride hydrochloride obtained in procedure 1a and 198 mg of the commercially available amine pyrrolidin-3-5-ylcarbamic acid tert-butyl ester, 379 mg of expected product are obtained.

MH+=529.2

Stage 2: N-2-[4-(3-R-Aminopyrrolidine-1-sulphonyl)phenyl]-N-4-(4-fluorophenyl)pyrimidine-2,4-diamine

According to the decarboxylation reaction described in stage 2 of procedure 3c, using 300 mg of product obtained in stage 1, 410 mg of expected product are obtained.

MH+=429.0

Melting point=250° C.

Procedure 3e: 4-Aminomethyl-1-{4-[4-(4-fluoro-3-methyl-phenylamino)pyrimidin-2-ylamino]benzenesulphonyl}-piperidin-4-ol

Stage 1: 6-Benzyl-1-oxa-6-azaspiro[2.5]octane

A solution of 3.2 g of sodium hydroxide in 32 ml of water is added dropwise to a solution containing 10 g of N-benzyl-4-piperidone, 12.8 g of dimethyloxosulphonium methylide and 0.34 g of tetrabutylammonium bromide in 100 ml of toluene, and the reaction medium is left to stir at 80° C. for 3 hours. After cooling, the mixture is washed with water and the product is dried over Na₂SO₄ and concentrated to dryness. 11.7 g of epoxide are thus obtained.

Stage 2: 4-Aminomethyl-1-benzylpiperidin-4-ol

6 g of epoxide obtained in stage 1 are solubilized in MeOH saturated with ammonia and heated for 72 hours in a sealed tube. The resulting product is concentrated under vacuum and purified on an alumina column (DCM-MeOH gradient; v/v; 9/1). 5.3 g of amino alcohol are obtained.

Stage 3: (1-Benzyl-4-hydroxypiperidin-4-ylmethyl)-carbamic acid tert-butyl ester

5.3 g of the amino alcohol obtained in stage 2 in solution in DCM are treated with 5.2 g of BOC₂O dissolved in DCM and the mixture is stirred for 15 minutes at AT. The resulting product is concentrated under vacuum and purified by alumina chromatography (DCM-MeOH gradient: v/v; 98/2). 5.2 g of the expected substituted amino alcohol are thus obtained.

Stage 4: (4-Hydroxypiperidin-4-ylmethyl)carbamic acid tert-butyl ester

According to a hydrogenolysis reaction, using 5.1 g of amino alcohol obtained in stage 2, in the presence of 510 mg of 10% Pd/C in 200 ml of methanol, 2.8 g of expected piperidine are obtained after the usual treatment.

Stage 5: 4-Aminomethyl-1-{4-[4-(4-fluoro-3-methylphenyl-amino)pyrimidin-2-ylamino]benzenesulphonyl}piperidin-4-ol

According to the protocol described in stage 1 of procedure 3a, using 700 mg of sulphonyl chloride hydrochloride obtained in procedure 1c and 420 mg of piperidine obtained in stage 4, 540 mg of a compound are obtained, which compound is subjected to a decarboxylation reaction to give 150 mg of expected sulphonamide.

MH+=487.1

Melting point=199° C.

1H NMR (DMSO):

1.47-1.77 (unresolved peak, 4); 2.25 (s, 3); 2.50 (m, 2); 2.75 (m, 2); 3.42 (m, 2); 4.99 (m, 1); 6.57 (d, 1); 7.20 (t, 1); 7.40 (m, 1); 7.60 (m, 1); 7.69 (d, 2); 7.83 (d, 2); 7.94 (ls, 3); 8.09 (d, 1); 10.92-11.23 (2ls, 2)

Procedure 3f: N-2-[4-(3-Aminomethylpyrrolidine-1-sulphonyl)phenyl]-N-4-(4-fluoro-3-methylphenyl)pyrimidine-2,4-diamine (racemic)

According to the protocol described in stage 1 of procedure 3a, using 2 g of sulphonyl chloride hydrochloride obtained in procedure 1c and 1.38 g of the racemic pyrrolidin-3-ylmethylcarbamic acid benzyl amine obtained in stage 4, 1.8 g of a compound are obtained, which compound is subjected to a hydrogenolysis reaction to give 1.3 g of expected sulphonamide.

Procedure 3 g: N-2-[4-(3-Aminomethylpiperidine-1-sulphonyl)phenyl]-N-4-(4-fluorophenyl)pyrimidine-2,4-diamine (racemic)

According to the protocol described in stage 1 of procedure 3a, using 3.5 g of sulphonyl chloride hydrochloride obtained in procedure 1a and 2 g of 3-N-boc-3-methylaminopiperidine, 2.65 g of a compound are obtained, which compound is subjected to a decarboxylation reaction to give 1.9 g of expected sulphonamide.

MH+=457.2

Mp=217-218° C.

Procedure 3h: N-2-[4-(4-Aminopiperidine-1-sulphonyl)-phenyl]-N-4-(3,4-difluorophenyl)pyrimidine-2,4-diamine (racemic)

According to the protocol described in stage 1 of procedure 3a, using 5 g of sulphonyl chloride hydrochloride obtained in procedure 1b and 2.41 g of 4-N-boc-4-aminopiperidine, 2.9 g of a compound are obtained, which compound is subjected to a decarboxylation reaction to give 2.9 g of expected sulphonamide.

MH+=443.2

Procedure 3i: N-2-[4-(3-Aminomethylpiperidine-1-sulphonyl)phenyl]-N-4-(4-fluoro-3-methylphenyl)pyrimidine-2,4-diamine (racemic)

According to the protocol described in stage 1 of procedure 3a, using 5.8 g of sulphonyl chloride hydrochloride obtained in procedure 1c and 2.996 g of 3-boc-aminomethylpiperidine, 4.1 g of a compound are obtained, which compound is subjected to a decarboxylation reaction to give 2.2 g of expected sulphonamide.

Procedure 4: Preparation of Reaction Intermediates of Carboxamide Type

Procedure 4a: {4-[4-(4-Fluoro-3-methylphenylamino)pyrimidin-2-ylamino]phenyl}-(4-methylaminopiperidin-1-yl)methanone

Stage 1: (1-{4-[4-(4-Fluoro-3-methylphenylamino)pyrimidin-2-ylamino]benzoyl}piperidin-4-yl)methyl-carbamic acid tert-butyl ester

A mixture containing 3 g of the acid obtained in procedure 2b, 1.9 g of 4-N-boc-4-methylaminopiperidine in the presence of 3.9 g of BOP and 4.5 ml of DIPEA in 30 ml of CH₂Cl₂ is reacted at AT overnight. The resulting product is evaporated to dryness, a 10% solution of potassium carbonate is added, and the mixture is extracted with ethyl acetate. After washing with water and drying of the organic phase over Na₂SO₄, filtration is carried out followed by chromatography on a silica column using, as eluent, DCM/MeOH (99/1; v/v). 3.85 g of expected product are obtained.

Stage 2: {4-[4-(4-Fluoro-3-methylphenylamino)pyrimidin-2-ylamino]phenyl}-(4-methylaminopiperidin-1-yl)methanone

The product obtained in stage 1 is dissolved in 40 ml of MeOH. 40 ml of 2N Et₂O are added at ambient temperature and the mixture is left to stir for 6 hours. After evaporation to dryness, the residue is triturated in Et₂O and filtration of the suspension generates 3.3 g of hydrochloride of expected product. The hydrochloride is dissolved in water, and basified with solid potassium carbonate. Extraction of this aqueous phase is carried out with ethyl acetate containing a small amount of THF. After washing and drying of the organic phase over Na₂SO₄, the product is evaporated to dryness and recrystallized from a DCM-iPr₂O mixture so as to obtain 2.25 g of expected product.

MH+=435.2

Mp=195-199° C.

NMR (1H, DMSO)

1.18 (m, 2); 1.80 (dl, 2); 2.23 (d, 3); 2.27 (s, 3); 2.54 (m, 1); 3.02 (t, 2); 3.66-4.34 (sl, 2); 6.22 (d, 1); 7.09 (t, 1); 7.27 (d, 2); 7.47 (m, 1); 7.60 (dd, 1); 7.79 (d, 2); 8.04 (d, 1); 9.35 (s, 1); 9.40 (s, 1).

Procedure 4b: {4-[4-(4-Fluorophenylamino)pyrimidin-2-ylamino]phenyl}-(3-methylaminopiperidin-1-yl)methanone (racemic)

Stage 1: {4-[4-(4-Fluorophenylamino)pyrimidin-2-ylamino]phenyl}-(3-methylaminopiperidin-1-yl)methanone (racemic)

According to the protocol described in stage 1 of procedure 4a, using 3.35 g of acid obtained in procedure 2a and 2 g of 3-N-boc-3-methylaminopiperidine, 3.7 g of expected compound are obtained.

Stage 2: {4-[4-(4-Fluorophenylamino)pyrimidin-2-ylamino]phenyl}-(3-methylaminopiperidin-1-yl)methanone (racemic)

According to the decarboxylation reaction described in stage 2 of procedure 4a, 3.7 g of compound obtained in stage 1 make it possible to obtain 2.8 g of expected carboxamide.

MH+=421.1

Melting point=110° C.

1H NMR (DMSO):

1.18-2.18 (unresolved peak, 5); 2.28 (s, 3); 2.41 (m, 1); 2.83 (m, 1); 3.08 (m, 1); 3.68-4.17 (2m, 2); 6.24 (d, 1); 7.14 (t, 2); 7.27 (d, 2); 7.68 (m, 2); 7.77 (d, 2); 8.04 (d, 1); 9.17 (s, 1); 9.24 (s, 1).

Procedure 4c: {4-[4-(4-Fluorophenylamino)pyrimidin-2-ylamino]phenyl}-(4-methylaminopiperidin-1-yl)methanone

Stage 1: (1-{4-[4-(4-Fluorophenylamino)pyrimidin-2-ylamino]benzoyl}piperidin-4-yl)methylcarbamic acid tert-butyl ester

According to the protocol described in stage 1 of procedure 4a, using 3.95 g of acid obtained in procedure 2a and 2.35 g of 3-N-boc-3-methylaminopiperidine, 4.3 g of expected compound are obtained. MH+=521.3

Stage 2: {4-[4-(4-Fluorophenylamino)pyrimidin-2-ylamino]phenyl}-(4-methylaminopiperidin-1-yl)methanone

According to the decarboxylation reaction described in stage 2 of procedure 4a, 4.3 g of the compound obtained in stage 1 make it possible to obtain 2.1 g of expected carboxamide.

Procedure 4d: (4-Methylaminopiperidin-1-yl)-{4-[4-(4-trifluoromethylphenylamino)pyrimidin-2-ylamino]phenyl}methanone

Stage 1: Methyl-(1-{4-[4-(4-trifluoromethylphenylamino)-pyrimidin-2-ylamino]benzoyl}piperidin-4-yl)carbamic acid tert-butyl ester

According to the protocol described in stage 1 of procedure 4a, using 1.5 g of acid obtained in procedure 2c and 1.7 g of 4-N-boc-4-methylaminopiperidine, 1.75 g of expected compound are obtained.

Stage 2: (4-Methylaminopiperidin-1-yl)-{4-[4-(4-trifluoromethylphenylamino)pyrimidin-2-ylamino]phenyl}methanone

According to the decarboxylation reaction described in stage 2 of procedure 4a, 1.75 g of compound obtained in stage 1 make it possible to obtain 248 mg of expected carboxamide.

MH+=470.9

Melting point=225-226° C.

Procedure 5:

Procedure 5a: 4-Pyrrolidin-1-ylmethylpiperidin-4-ol

Step 1: 1-Oxa-6-azaspiro[2.5]octane-6-carboxylic acid tert-butyl ester

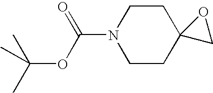

18.22 g of trimethylsulphoxonium iodide and 485 mg of tetrabutylammonium bromide are added to a suspension of 15 g of 4-oxopiperidine-1-carboxylic acid tert-butyl ester in 150 ml of toluene. A solution of 4.5 g of sodium hydroxide in 20 ml of water is added dropwise. The mixture is left to stir for 3 h at 80° C. It is taken up with toluene, the mixture is separated by settling out, washing is carried out with water, and the product is dried and concentrated to dryness. After silica column chromatography (DCM/EtOAc:90/10), 13 g of expected product are obtained.

Step 2: 4-Hydroxy-4-pyrrolidin-1-ylmethylpiperidine-1-carboxylic acid tert-butyl ester

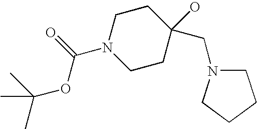

2.2 g of product obtained in the preceding step are solubilized with 1.46 g of pyrrolidine and 25 ml of EtOH in a sealed tube. The reaction medium is heated at 75° C. for 18 h. After concentration to dryness, taking up with water, extraction with DCM, drying and concentration, 2.9 g of desired product are obtained.

Step 3: 4-Pyrrolidin-1-ylmethylpiperidin-4-ol dihydrochloride

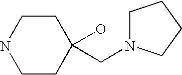

2.9 g of product above, in the presence of a 4M solution of HCl in dioxane, are stirred for 4 h at AT in a dioxane-MeOH mixture (50 ml). The resulting mixture is concentrated under vacuum and the product is triturated in isopropyl ether, and the solid is filtered off and used as it is in the coupling reaction with sulphonyl chloride.

Procedure 5b: 4-(2-Methylpyrrolidin)-1-ylmethylpiperidin-4-ol

This compound is synthesized according to the scheme described in procedure 5a, replacing the pyrrolidine with 2-methylpyrrolidine in step 2.

Procedure 5c: 4-(3-Methylpyrrolidin)-1-ylmethylpiperidin-4-ol

This compound is synthesized according to the scheme described in procedure 5a, replacing the pyrrolidine with 3-methylpyrrolidine in step 2.

Procedure 5d: 4-(2-R-Methylpyrrolidin)-1-ylmethylpiperidin-4-ol

This compound is synthesized according to the scheme described in procedure 5a, replacing the pyrrolidine with 2-R-methylpyrrolidine in step 2.

Procedure 5e: 4-(2-S-Methylpyrrolidin)-1-ylmethylpiperidin-4-ol

This compound is synthesized according to the scheme described in procedure 5a, replacing the pyrrolidine with 2-S-methylpyrrolidine in step 2.

Procedure 5 f: 4-(Azetidin)-1-ylmethylpiperidin-4-ol

This compound is synthesized according to the scheme described in procedure 5a, replacing the pyrrolidine with azetidine in step 2.

### Example 1

### {4-[4-(4-Fluoro-3-methylphenylamino)pyrimidin-2-ylamino]phenyl}-[4-(methyl-[1,2,3]thiadiazol-4-ylmethylamino)piperidin-1-yl]methanone

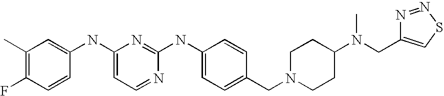

370 mg of acid of procedure 4a and 95 mg of 1,2,3-thiadiazole-4-carboxaldehyde are mixed in THF (10 ml) and 310 mg of NaBH(OAc)₃ are added. The mixture is stirred at ambient temperature overnight, CH₃OH (5 ml) is added and the resulting mixture is heated at 60° C. for one hour. After evaporation of the solvents, water is added and the product is basified with a few drops of sodium hydroxide and extracted with CH₂Cl₂, followed by the usual treatment and silica gel chromatography. Elution is carried out with CH₂Cl₂/CH₃OH (98/2; v/v). Recrystallization from CH₂Cl₂-i(Pr)₂O is carried out, so as to obtain 225 mg.

MH+=533.2

Mp=183-184° C.

NMR (1H, DMSO)

1.49 (qd, 2); 1.85 (dl, 2); 2.24 (s, 3); 2.24 (s, 3); 2.67 (t, 1); 2.77-3.09 (unresolved peak, 2); 3.67-4.77 (unresolved peak, 2); 4.16 (s, 2); 6.22 (d, 1); 7.09 (t, 1); 7.30 (d, 2); 7.46 (m, 1); 7.59 (dd, 1); 7.78 (d, 2); 8.03 (d, 1); 9.02 (s, 1); 9.34 (s, 1); 9.38 (s, 1).

### Example 2

### {4-[4-(4-Fluorophenylamino)pyrimidin-2-ylamino]phenyl}-{4-[methyl-(1H-pyrazol-4-ylmethyl)-amino]piperidin-1-yl}methanone

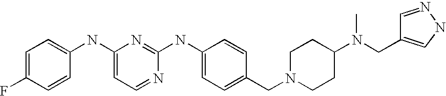

In the same manner as in Example 1, using 400 mg of the acid of procedure 4c and 1H-pyrazole-4-carboxaldehyde (100 mg), the expected product is obtained.

MH+=501.5

Mp=140-145° C.

NMR (1H, DMSO)

1.44 (m, 2); 1.78 (m, 2); 2.14 (s, 3); 2.59 (m, 1); 2.86 (lm, 2); 3.52 (ls, 2); 3.65-4.71 (unresolved peak, 2); 6.23 (d, 1); 7.16 (t, 2); 7.28 (d, 2); 7.33-7.63 (unresolved peak, 2); 7.7 (m, 2); 7.81 (d, 2); 8.04 (m, 1); 9.39 (2s, 2); 12.64 (ls, 1).

### Example 3

### {4-[4-(4-Fluorophenylamino)pyrimidin-2-ylamino]phenyl}-{4-[methyl-(1H-pyrazol-3-ylmethyl)-amino]piperidin-1-yl}methanone

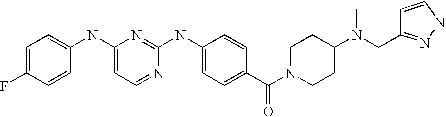

In the same manner as in Example 1, using 400 mg of the acid of procedure 4c and 100 mg of 1H-pyrazole-3-carboxaldehyde. The expected product is obtained.

MH+=501.3

Mp=165-170° C.

NMR (1H, DMSO)

1.44 (m, 2); 1.78 (m, 2); 2.14 (s, 3); 2.59 (m, 1); 2.86 (lm, 2); 3.91 (ls, 2); 3.65-4.71 (unresolved peak, 2); 6.12 (s, 1); 6.23 (d, 1); 7.16 (t, 2); 7.28 (d, 2); 7.33-7.63 (unresolved peak, 1); 7.7 (m, 2); 7.81 (d, 2); 8.04 (m, 1); 9.39 (2s, 2); 12.64 (ls, 1)

### Example 4

### {4-[4-(4-Fluorophenylamino)pyrimidin-2-ylamino]phenyl}-{4-[methyl-(1H-pyrazol-4-ylmethyl)-amino]piperidin-1-yl}methanone

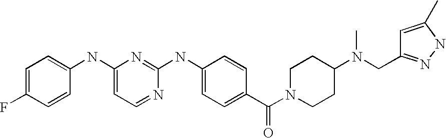

In the same manner as in Example 1, using 400 mg of the acid of procedure 4c and 100 mg of 3-methyl-1H-pyrazole-5-carboxaldehyde. The expected product is obtained.

MH+=515.4

Mp=214-215° C.

NMR (1H, DMSO)

1.47 (m, 2); 1.82 (m, 2); 2.19 (s, 6); 2.63 (m, 1); 2.91 (t, 2); 3.56 (ls, 2); 4.11 (lm, 2); 5.9 (s, 1); 6.24 (d, 1); 7.14 (t, 2); 7.28 (d, 2); 7.67 (m, 2); 7.7 (m, 2); 8.04 (d, 1); 9.12 (s, 1); 9.20 (2s, 1); 11.97 (ls, 1)

### Example 5

### [4-(Benzo[1,2,5]thiadiazol-5-ylmethyl-methylamino)piperidin-1-yl]-{4-[4-(4-fluorophenyl-amino)pyrimidin-2-ylamino]phenyl}methanone

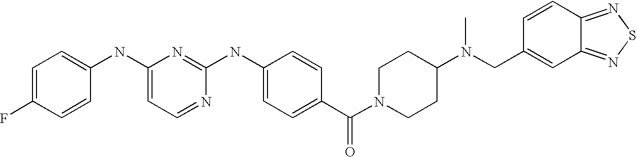

In the same manner as in Example 1, using 420 mg of the acid of procedure 4c and 164 mg of 2,1,3-benzothiadiazole-5-carbaldehyde. 242 mg of expected product are obtained. MH+=569.1

Melting point=191-192° C.

### Example 6

### {4-[4-(4-Fluorophenylamino)pyrimidin-2-ylamino]phenyl}-[4-(methyloxazol-2-ylmethylamino)piperidin-1-yl]methanone

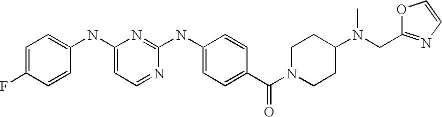

In the same manner as in Example 1, using 420 mg of the acid of procedure 4c and 110 mg of oxazole-2-carbaldehyde, 380 mg of expected product are obtained.

MH+=501.9

Melting point=175-176° C.

1H NMR (DMSO):

1.39 (m, 2); 1.79 (dl, 2); 2.23 (s, 3); 2.62 (m, 1); 2.88 (sl, 2); 3.77 (s, 2); 4.02 (sl, 2); 6.22 (d, 1); 7.05-7.23 (unresolved peak, 3); 7.23 (d, 2); 7.71 (m, 2); 7.78 (d, 2); 8.00-8.11 (unresolved peak, 2); 9.40 (s, 1); 9.43 (s, 1).

### Example 7

### {4-[4-(4-Fluorophenylamino)pyrimidin-2-ylamino]phenyl}-[3-(methyloxazol-2-ylmethylamino)piperidin-1-yl]methanone (racemic)

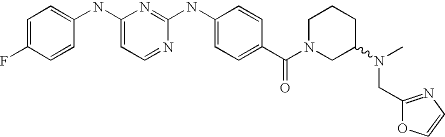

In the same manner as in Example 1, using 400 mg of the acid of procedure 4b and 100 mg of oxazole-2-carbaldehyde, 279 mg of expected product are obtained.

MH+=501.9

Melting point=155-157° C.

1H NMR (DMSO):

1.41 (m, 1); 1.54 (m, 1); 1.72 (m, 1); 1.93 (m, 1); 2.25 (ls, 3): 2.51 (m, 1); 2.92 (ls, 2); 3.49-4.75 (unresolved peak, 4); 6.24 (d, 1); 7.16 (unresolved peak, 2); 7.27 (d, 2); 7.71 (m, 2); 7.79 (d, 2); 8.04 (unresolved peak, 3); 9.38 (s, 1); 9.42 (s, 1).

### Example 8

### {4-[4-(4-Fluorophenylamino)pyrimidin-2-ylamino]phenyl}-[3-(methyl-1H-pyrrole-2-ylmethylamino)-piperidin-1-yl]methanone (racemic)

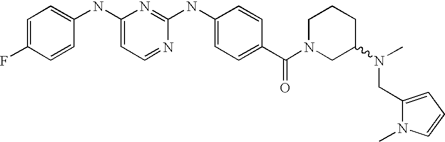

In the same manner as in Example 1, using 400 mg of the acid of procedure 4b and 100 mg of 1-methyl-1H-pyrrole-2-carbaldehyde, 102 mg of expected product are obtained.

MH+=513.9

Melting point=148-151° C.

1H NMR (DMSO):

1.40 (m, 1); 1.60 (m, 1); 1.74 (m, 1); 1.91 (m, 1); 2.10 (ls, 3); 2.54 (m, 1); 2.59-3.14 (unresolved peak, 2); 3.23 (s, 3); 3.38-4.76 (unresolved peak, 4); 5.86 (ls, 2); 6.23 (d, 1); 6.63 (s, 1); 7.16 (t, 2); 7.23 (m, 2); 7.72 (m, 2); 7.78 (d, 2); 8.06 (d, 1); 9.37 (s, 1); 9.42 (s, 1).

### Example 9

### {4-[4-(4-Fluorophenylamino)pyrimidin-2-ylamino]phenyl}-[3-(aminothiazol-2-ylmethylamino)piperidin-1-yl]methanone (racemic)

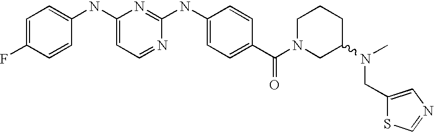

In the same manner as in Example 1, using 340 mg of the acid of procedure 4b and 100 mg of 1-methyl-1H-pyrrole-2-carbaldehyde, 289 mg of expected product are obtained.

MH+=518.0

Melting point=160-165° C.

1H NMR (DMSO):

1.18-1.99 (unresolved peak, 4); 2.18 (sl, 3); 2.54 (m, 1); 2.94 (sl, 2); 3.86 (sl, 2); 4.17 (sl, 2); 6.23 (d, 1); 7.17 (t, 2); 7.27 (d, 2); 7.61-7.89 (unresolved peak, 5); 8.06 (d, 1); 9.00 (s, 1); 9.41 (s, 1); 9.45 (s, 1).

### Example 10

### {4-[4-(4-Fluorophenylamino)pyrimidin-2-ylamino]phenyl}-{3-[methyl-(1H-pyrazol-3-ylmethyl)-amino]piperidin-1-yl}methanone (racemic)

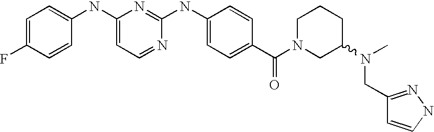

In the same manner as in Example 1, using 340 mg of the acid of procedure 4b and 120 mg of 1H-pyrazole-3-carbaldehyde, 267 mg of expected product are obtained.

MH+=501.0

Melting point=120-140° C.

1H NMR (DMSO):

1.39 (m, 1); 1.53 (m, 1); 1.73 (sl, 1); 1.95 (dl, 1); 2.16 (sl, 3); 2.48 (m, 1); 2.90 (sl, 2); 3.61 (sl, 2); 4.32 (sl, 2); 6.09 (sl, 1); 6.23 (d, 1); 7.16 (t, 2); 7.26 (d, 2); 7.51 (sl, 1); 7.71 (m, 2); 7.79 (d, 2); 8.05 (d, 1); 9.38 (s, 1); 9.41 (s, 1).

### Example 11

### N*4*-(4-Fluoro-3-methylphenyl)-N*2*-(4-{4-[(2-methanesulphonylethyl)methylamino]piperidine-1-sulphonyl}phenyl)pyrimidine-2,4-diamine

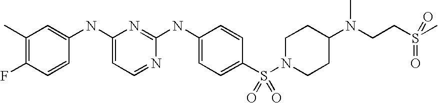

250 mg of TEA and then 100 mg of methylvinylsulphone are added to a suspension of 300 mg of the compound obtained in procedure 3a in 18 ml of an MeOH/DMF mixture (v/v; 5/1). After stirring at AT for 18 hours, concentration to dryness, taking up in DCM, washing with H₂O and drying, the resulting product is again concentrated to dryness. Chromatography (SiO₂) is performed, elution being carried out with DCM/MeOH (v/v; 94/6), the resulting product is recrystallized from iPR₂O, and 220 mg of expected product are obtained.

MH+=577.1

Mp=115° C.

NMR (1H, DMSO)

1.47 (m, 2); 1.69 (m, 2); 2.12 (s, 3); 2.24 (unresolved peak, 5); 2.33 (m, 1); 2.76 (t, 2); 2.93 (s, 3); 3.17 (t, 2); 3.60 (ld, 2); 6.27 (d, 1); 7.10 (t, 1); 7.47 (m, 1); 7.57 (m, 3); 7.97 (d, 2); 8.07 (d, 1); 9.41 (s, 1); 9.69 (s, 1)

### Example 12

### 2-[(1-{4-[4-(4-Fluoro-3-methylphenylamino)-pyrimidin-2-ylamino]benzenesulphonyl}piperidin-4-yl)-methylamino]-N,N-dimethylacetamide

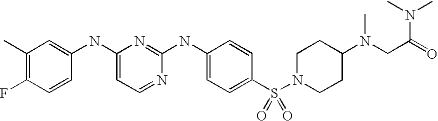

A mixture of 400 mg of the compound obtained in procedure 3a, 120 mg of 2-chloro-N,N-dimethylacetamide, 160 mg of KI and 260 mg of K₂CO₃ is stirred at ambient temperature overnight in CH₃CN (10 ml). After extraction in the usual manner, the residue is chromatographed on silica, elution being carried out with CH₂Cl₂/CH₃OH 94/6; v/v). Recrystallization is carried out from CH₂Cl₂-i(Pr)₂O, so as to obtain 126 mg.

MH+=556.2

Mp=215-218° C.

NMR (1H, DMSO)

1.45 (qd, 2); 1.71 (dd, 2); 2.12 (s, 3); 2.18 (m, 2); 2.24 (s, 3); 2.38 (m, 1); 2.74 (s, 3); 2.92 (s, 3); 3.15 (s, 2); 3.59 (d, 2); 6.29 (d, 1); 7.11 (t, 1); 7.47 (m, 1); 7.58 (d, 2); 7.58 (m, 1); 7.98 (d, 2); 8.08 (d, 1); 9.43 (s, 1); 9.72 (s, 1).

### Example 13

### 3-[(1-{4-[4-(4-Fluoro-3-methylphenyl-amino)pyrimidin-2-ylamino]benzenesulphonyl}piperidin-4-yl)methylamino]-N,N-dimethylpropionamide

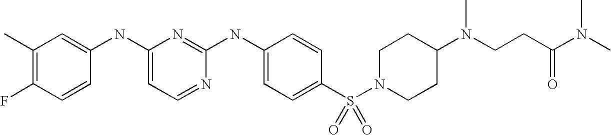

TEA (0.35 ml) and then N,N-dimethylacrylamide (0.1 ml) are added to a suspension of 400 mg of the compound obtained in procedure 3a in 20 ml of ethanol. The mixture is heated at 90° C. for 12 hours and then left to stir at AT for 48 hours. The mixture is evaporated to dryness and EtOAc is added. After treatment, chromatography (SiO₂) is performed, elution being carried out with DCM/MeOH (v/v; 93/7), and recrystallization is carried out from DCM/iPr₂O, so as to obtain 85 mg of expected product.

MH+=570.3

Mp=201° C.

1H NMR (DMSO):

1.42 (qd, 2); 1.69 (dd, 2); 2.10 (s, 3); 2.24 (s, 3); 2.24 (d, 2); 2.29 (m, 1); 2.33 (t, 2); 2.58 (t, 2); 2.75 (s, 3); 2.91 (s, 3); 3.59 (d, 2); 6.29 (d, 1); 7.11 (t, 1); 7.47 (m, 1); 7.58 (d, 2); 7.58 (d, 1); 7.98 (d, 2); 8.08 (d, 1); 9.42 (sl, 1); 9.70 (sl, 1).

### Example 14

### 1-{4-[4-(4-Fluorophenylamino)pyrimidin-2-ylamino]benzoyl}-3-pyridin-3-ylmethylpiperidine-3-carboxylic acid ethyl ester (racemic)

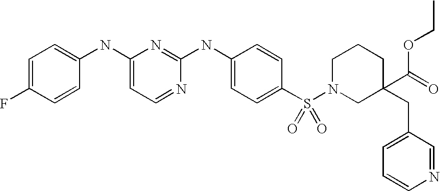

According to the protocol described in stage 1 of procedure 3a, using 3 g of benzoic acid obtained in procedure 2a and 2.25 g of commercially available amine, 2.5 mg of expected product are obtained.

MH+=605.2

Melting point=119° C.

1H NMR (DMSO):

1.12 (t, 3); 1.26-1.85 (unresolved peak, 4); 2.25 (dl, 3); 2.45 (m, 2); 2.66-2.98 (dd, 2); 3.17 (dl, 1); 3.60 (d, 1); 4.03 (m, 2); 6.29 (d, 1); 7.12 (t, 1); 7.31 (m, 1); 7.39-7.65 (unresolved peak, 5); 8.00 (d, 2); 8.08 (d, 1); 8.28 (m, 1); 8.43 (m, 1); 9.43 (s, 1); 9.73 (s, 1).

### Example 15

### 1-{4-[4-(4-Fluorophenylamino)pyrimidin-2-ylamino]benzoyl}-3-pyridin-3-ylmethylpiperidine-3-carboxylic acid dimethylamide (racemic)

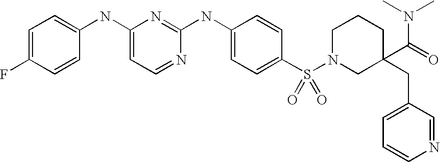

Stage 1: 660 mg of KOH are dissolved in 5 ml of water. 3.5 g of ester obtained in Example 14 and 50 ml of MeOH are added to this solution. After refluxing for 3 hours, the reaction medium is concentrated to dryness and taken up with water acidified to a pH of 7. The precipitate formed is filtered off.

Stage 2: The acid (500 mg) obtained in stage 1 is reacted with 187 mg of EDC, 138 mg of HOBT, 150 mg of dimethylamide hydrochloride and 230 mg of DIPEA. After reaction for 18 hours, the reaction medium is concentrated under vacuum, and the resulting product is taken up with DCM, washed with water, dried and concentrated under vacuum. The crude product is purified by silica column chromatography, elution being carried out with a DCM-MeOH mixture (v/v, 98/2). 290 mg of expected carboxamide are obtained.

MH+=590.2

Melting point=146° C.

1H NMR (DMSO):

1.53-1.85 (unresolved peak, 4); 2.56-3.28 (unresolved peak, 12); 6.30 (d, 1); 7.19 (t, 2); 7.29 (m, 1); 7.50 (m, 1); 7.60 (d, 2); 7.71 (m, 2); 8.01 (d, 2); 8.10 (d, 1); 8.32 (dl, 1); 8.43 (ddI, 1); 9.51 (s, 1); 9.74 (s, 1).

### Example 16

### 1-{4-[4-(4-Fluorophenylamino)pyrimidin-2-ylamino]benzoyl}-3-pyridin-3-ylmethylpiperidine-3-carboxylic acid methylamide (racemic)

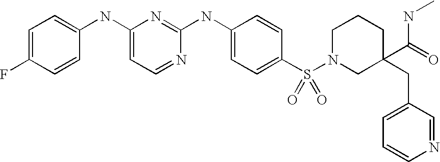

According to the protocol described in stage 2 of Example 15, using 500 mg of the acid obtained in stage 1 of Example 15 and 120 mg of monomethylamine hydrochloride, 255 mg of expected carboxamide are obtained.

MH+=576.2

Melting point=150° C.

1H NMR (DMSO):

1.3-1.82 (unresolved peak, 4); 2.5 (s, 3); 2.58-3.13 (unresolved peak, 6); 6.25 (d, 1); 7.13 (t, 2); 7.26 (m, 1); 7.40 (m, 1); 7.54 (d*, 3); 7.66 (m, 2); 7.96 (d, 2); 8.04 (d, 1); 8.22 (d, 1); 8.38 (m, 1); 9.47 (s, 1); 9.68 (s, 1).

### Example 17

### 1-{4-[4-(4-Fluorophenylamino)pyrimidin-2-ylamino]-benzoyl}-3-pyridin-3-ylmethylpiperidine-3-carboxylic acid (2-dimethylaminoethyl)amide (racemic)

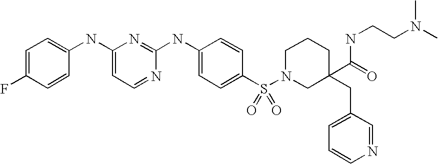

According to the protocol described in Example 16, using 500 mg of the acid obtained in stage 1 of Example 15 and 118 mg of N,N-dimethylaminoethylamine, 220 mg of expected carboxamide are obtained.

MH+=633.4

Melting point=122° C.

1H NMR (DMSO):

1.22-1.82 (unresolved peak, 4); 2.08 (s, 6); 2.18 (t, 2); 2.58-3.18 (unresolved peak, 8); 6.25 (d, 1); 7.15 (t, 2); 7.25 (m, 1), 7.52 (2m, 4); 7.66 (m, 2); 7.95 (d, 2); 8.04 (d, 1); 8.29 (d, 1); 8.37 (d, 1); 9.48 (s, 1); 9.69 (s, 1).

### Example 18

### N*4*-(4-Fluorophenyl)-N*2*-[4-(3-{[(1-methyl-1H-pyrrol-2-ylmethyl)amino]methyl}piperidine-1-sulphonyl)phenyl]pyrimidine-2,4-diamine (racemic)

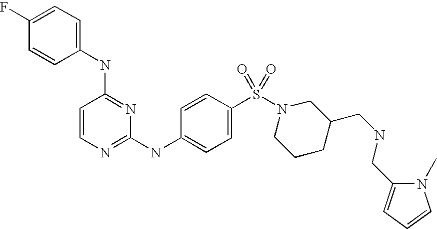

In the same manner as in Example 1, using 400 mg of the compound of procedure 3b and 102 mg of 1-methyl-1H-pyrrole-2-carbaldehyde, 202 mg of expected product are obtained.

MH+=550.1.1

Melting point=184.2° C.

1H NMR (DMSO):

0.80 (m, 1); 1.18-1.97 (unresolved peak, 6); 1.98-2.41; (unresolved peak, 3); 3.35-3.71 (unresolved peak, 7); 5.80 (d, 2); 6.25 (d, 1); 6.57 (t, 1); 7.14 (t, 2); 7.52 (d, 2); 7.67 (m, 2); 7.95 (d, 2); 8.05 (d, 1); 9.47 (s, 1); 9.67 (s, 1).

### Example 19

### N*4*-(4-Fluorophenyl)-N*2*-[4-(3-{[(5-methyl-isoxazol-3-ylmethyl)amino]methyl}piperidine-1-sulphonyl)-phenyl]pyrimidine-2,4-diamine (racemic)

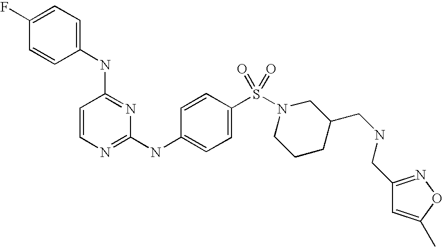

In the same manner as in Example 1, using 500 mg of the compound of procedure 3b and 140 mg of 5-methylisoxazole-3-carbaldehyde, 443 mg of expected product are obtained.

MH+=552.2

Melting point=95° C.

1H NMR (DMSO):

0.79 (m, 1); 1.22-1.71 (unresolved peak, 4); 1.83 (t, 1); 1.99-2.39 (unresolved peak, 7); 3.41 (d, 1); 3.58 (unresolved peak, 3); 6.13 (s, 1); 6.24 (d, 1); 7.13 (t, 2); 7.53 (d, 2); 7.66 (m, 2); 7.94 (d, 2); 8.05 (d, 1); 9.46 (s, 1); 9.65 (s, 1).

### Example 20

### N*4*-(4-Fluorophenyl)-N*2*-[4-(3-{[(thiophen-2-ylmethyl)amino]methyl}piperidine-1-sulphonyl)phenyl]pyrimidine-2,4-diamine (racemic)

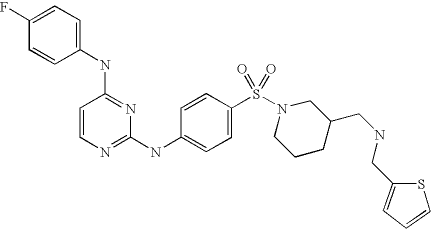

In the same manner as in Example 1, using 500 mg of the compound of procedure 3b and 138 mg of 1-methyl-1H-pyrrole-2-carbaldehyde, 200 mg of expected product are obtained.

MH+=553.2

Melting point=98.8° C.

1H NMR (DMSO):

0.77 (m, 1); 1.10-1.94 (unresolved peak, 5); 1.95-2.41 (unresolved peak, 4); 3.41 (d, 1); 3.63 (d, 1); 3.79 (s, 2); 6.25 (d, 1); 6.89 (unresolved peak, 2); 7.13 (t, 2); 7.31 (m, 1); 7.53 (d, 2); 7.66 (m, 2); 7.94 (d, 2); 8.04 (d, 1); 9.47 (s, 1); 9.67 (s, 1).

### Example 21

### N*4*-(4-Fluorophenyl)-N*2*-(4-{3-[(2-methanesulphonylethylamino)methyl]piperidine-1-sulphonyl}phenyl)pyrimidine-2,4-diamine (racemic)

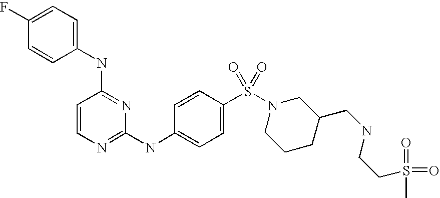

According to the addition reaction described in Example 11, using 300 mg of the compound of procedure 3b and 62 mg of vinylmethylsulphone, 294 mg of expected product are obtained.

MH+=563.1

Melting point=184.2° C.

1H NMR (DMSO):

0.86 (m, 1); 1.30-1.78 (unresolved peak, 4); 1.94 (2m, 2); 2.09-2.45 (unresolved peak, 3); 1.91 (m, 2); 3 (s, 3); 3.19 (t, 2); 3.47 (d, 1); 3.6 (d, 1); 6.30 (d, 1); 7.21 (t, 2); 7.58 (d, 2); 7.72 (m, 2); 7.99 (d, 2); 8.10 (d, 1); 9.51 (s, 1); 9.71 (s, 1).

### Example 22

### 4-Dimethylaminomethyl-1-{4-[4-(4-fluoro-3-methylphenylamino)pyrimidin-2-ylamino]benzenesulphonyl}-piperidin-4-ol

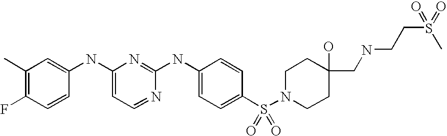

According to the addition reaction described in Example 11, using 290 mg of compound of procedure 3e and 82 mg of vinylmethylsulphone, 130 mg of expected product are obtained.

MH+=593.1

Melting point=233° C.

1H NMR (DMSO):

0.65 (m, 4); 2.24 (d, 3); 2.55 (m, 2); 2.93 (sl, 2); 3.04 (s, 3); 3.21-3.67 (unresolved peak, 6); 5.11 (sl, 1); 6.31 (d, 1); 7.11 (t, 1); 7.47 (m, 1); 7.53-7.66 (unresolved peak, 3); 8.01 (d, 2); 8.08 (d, 1); 8.75 (sl, 2); 9.51 (s, 1); 9.76 (s, 1).

### Example 23

### 1-{4-[4-(4-Fluoro-3-methylphenylamino)pyrimidin-2-ylamino]benzenesulphonyl}-4-imidazol-1-ylmethylpiperidin-4-ol

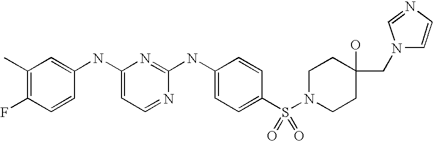

Stage 1: 1.2 equivalents of sodium hydride are added to a solution containing 290 mg of imidazole in 5 ml of DMSO. After stirring at AT for 20 minutes, 700 mg of epoxide obtained in stage 1 of procedure 3e are added and the mixture is left to stir for 18 hours at AT. The resulting product is taken up with water, the mixture is extracted with DCM, and the resulting product is dried over Na₂SO₄ and concentrated. 540 mg of expected alcohol are obtained by trituration.

Stage 2: According to a hydrogenolysis reaction described in stage 4 of procedure 3e, using 540 mg of alcohol obtained in stage 1, 280 mg of expected piperidine are obtained.

Stage 3: According to the protocol described in stage 1 of procedure 3a, using 600 mg of sulphonyl chloride hydrochloride and 280 mg of piperidine obtained in stage 2, 330 mg of expected sulphonamide are obtained.

MH+=538.2

Melting point=220° C.

1H NMR (DMSO):

1.35 (d, 2); 1.56 (m, 2); 2.24 (s, 3); 2.41 (t, 2); 3.4 (d, 2); 3.88 (s, 2); 4.66 (s, 1); 6.28 (d, 1); 6.84 (s, 1); 7.06 (s, 1); 7.10 (t, 1); 7.46 (m, 1); 7.49 (s, 1); 7.52-7.61 (unresolved peak, 3); 7.97 (d, 2); 8.07 (d, 1); 9.42 (s, 1); 9.17 (s, 1).

### Example 24

### N*4*-(4-Fluoro-3-methylphenyl)-N*2*-(4-{3-[(2-methanesulphonylethylamino)methyl]pyrrolidine-1-sulphonyl}phenyl)pyrimidine-2,4-diamine (racemic)

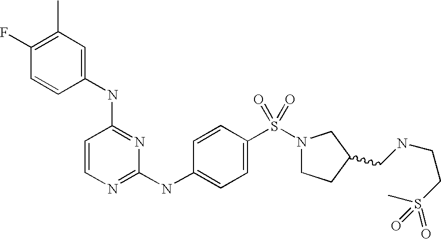

According to the addition reaction described in Example 11, using 400 mg of the compound of procedure 3f and 140 mg de vinylmethylsulphone, 260 mg of expected product are obtained.

MH+=563.2

Melting point=154° C.

### Example 25

### N*4*-(4-Fluoro-3-methylphenyl)-N*2*-[4-(3-{[(1-methyl-1H-pyrrol-2-ylmethyl)amino]methyl}-pyrrolidine-1-sulphonyl)phenyl]pyrimidine-2,4-diamine (racemic)

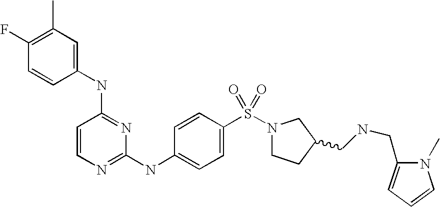

In the same manner as in Example 1, using 300 mg of the compound of procedure 3f and 83 mg of 1-methyl-1H-pyrrole-2-carbaldehyde, 100 mg of expected product are obtained.

MH+=550.0

Melting point=93° C.

1H NMR (DMSO):

1.39 (m, 1); 1.80 (m, 1); 2.10 (m, 1); 2.16-2.38 (unresolved peak, 5); 2.76-3.34 (unresolved peak, 4); 3.49 (s, 2); 3.51 (s, 3); 5.79 (unresolved peak, 2); 6.28 (d, 1); 6.57 (s, 1); 7.11 (t, 1); 7.46 (m, 1); 7.58 (d, 1); 7.63 (d, 2); 7.98 (d, 2); 8.07 (d, 1); 9.41 (s, 1); 9.69 (s, 1).

### Example 26

### {4-[4-(4-Fluorophenylamino)pyrimidin-2-ylamino]phenyl}-[3-(5-methylisoxazol-3-ylmethylamino)piperidin-1-yl]methanone (racemic)

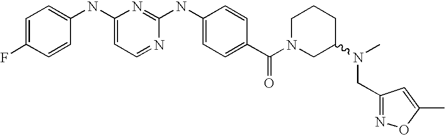

In the same manner as in Example 1, using 340 mg of the acid of procedure 4 g and 100 mg of 5-methylisoxazole-3-carbaldehyde, 360 mg of expected product are obtained.

MH+=516.0

Melting point=190-191° C.

1H NMR (DMSO):

1.43 (m, 1); 1.56 (m, 1); 1.75 (m, 1); 1.95 (dl, 1); 2.23 (s, 3); 2.37 (s, 3); 2.54 (m, 1); 2.94 (m, 2); 3.63 (sl, 2); 3.94 (sl, 1); 4.13 (sl, 1); 6.06 (s, 1); 6.24 (d, 1); 7.12 (t, 2); 7.26 (d, 2); 7.66 (m, 2); 7.77 (d, 2); 8.03 (d, 1); 9.06 (s, 1); 9.16 (s, 1).

### Example 27

### N*4*-(4-Fluorophenyl)-N*2*-(4-{3-[(2-methanesulphonylethyl)methylamino]piperidine-1-sulphonyl}phenyl)pyrimidine-2,4-diamine

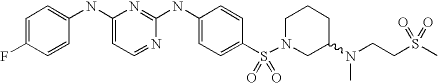

According to the addition reaction described in Example 11, using 400 mg of the compound of procedure 3 g and 140 mg of vinylmethylsulphone, 360 mg of expected compound are obtained.

MH+=563.1

Mp=103° C.

NMR (1H, DMSO)

1.25 (t, 6); 1.65 (m, 2); 2.09 (m, 2); 2.14-2.37 (unresolved peak, 4); 2.86-3.24 (2m, 3); 3.74 (d, 2); 4.02 (m, 4) 6.54 (d, 1); 7.26 (t, 2); 7.63 (m, 2); 7.68 (d, 2); 7.81 (d, 2); 8.09 (d, 1); 9.33 (ls, 2); 10.74-11.13 (2ls, 2).

### Example 28

### N*4*-(4-Fluorophenyl)-N*2*-{4-[4-(2-methanesulphonylethylamino)piperidine-1-sulphonyl]phenyl}pyrimidine-2,4-diamine

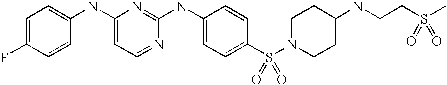

According to the addition reaction described in Example 11, using 800 mg of the compound of procedure 3h and 190 mg of vinylmethylsulphone, 340 mg of expected product are obtained.

MH+=549.2

Mp=157° C.

NMR (1H, DMSO)

1.24 (m, 2); 1.76 (m, 2); 2.38 (unresolved peak, 3); 2.79 (t, 2); 2.85 (s, 3); 3.08 (m, 2); 3.33 (m, 2); 6.25 (d, 1); 7.13 (t, 2); 7.53 (d, 2); 7.66 (m, 2); 7.92 (d, 2); 8.03 (d, 1); 9.45 (s, 1); 9.66 (s, 1).

### Example 29

### [3-(1-{4-[4-(3,4-Difluorophenylamino)pyrimidin-2-ylamino]benzenesulphonyl}piperidin-4-ylamino-methyl)ethyl]phosphonic acid diethyl ester

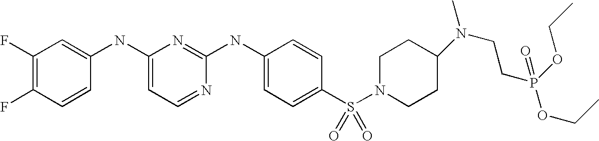

Stage 1: [2-(1-Benzylpiperidin-4-ylamino)ethyl]phosphonic acid diethyl ester

A mixture of 4-amino-1-benzylpiperidine (5 g) and (2-bromoethyl)phosphonic acid diethyl ester (7 g) is brought to reflux in EtOH (50 ml). After stirring at AT for 18 hours, the solid is filtered off and, after concentration, chromatography (Al₂O₃) is performed, elution being carried out with DCM/MeOH (v/v; 85/15) and 62 g of expected product are obtained.

Stage 2: {2-[(1-Benzylpiperidin-4-yl)methylamino]ethyl}-phosphonic acid diethyl ester

NaBH(OAC)₃ (1.6 g) is added to a mixture of compound (2 g) obtained in the stage 1 and formaldehyde (0.6 ml, 37% aqueous solution) in DCM (70 ml). After stirring for one hour and treatment with a solution of Na₂CO₃, extraction with DCM, drying and concentration, 1.9 g of expected product are obtained.

Stage 3: [2-(Methylpiperidin-4-ylamino)ethyl]phosphonic acid diethyl ester

1.2 g of 1-H-piperidine derivative are obtained by means of a hydrogenolysis reaction on 1.9 g of the compound obtained in stage 2.

Stage 4: [3-(1-{4-[4-(3,4-Difluorophenylamino)pyrimidin-2-ylamino]benzenesulphonyl}piperidin-4-ylaminomethyl)-ethyl]phosphonic acid diethyl ester

According to the protocol described in stage 1 of Example 30, 500 mg of difluoro derivative of procedure 2b and 420 mg of compound obtained in stage 3 make it possible to obtain 380 mg of the expected compound.

MH+=639

Mp=164° C.

NMR (1H, DMSO)

1.07-1.32 (unresolved peak, 8); 1.52 (q, 2); 1.71-2.02 (unresolved peak, 4); 2.41 (m, 2); 2.64 (s, 3); 2.80 (d, 2); 3.60 (m, 1); 3.95 (q, 4); 6.28 (d, 1); 7.17 (t, 2); 7.55-7.79 (unresolved peak, 4); 7.95 (d, 2); 8.08 (d, 1); 9.48 (s, 1) 9.67 (s, 1).

### Example 30

### [2-(1-{4-[4-(3,4-Difluorophenylamino)pyrimidin-2-ylamino]benzenesulphonyl}piperidin-4-ylamino)ethyl]phosphonic acid diethyl ester

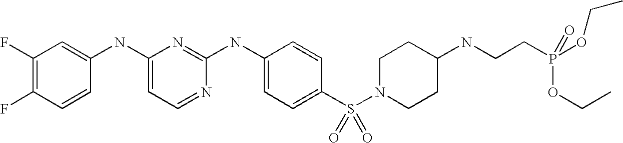

Stage 1: {2-[(1-Benzylpiperidin-4-yl)tert-butoxycarbonyl-amino]ethyl}phosphonic acid diethyl ester

2 g of BOC₂O dissolved in CH₃CN (16 ml) are added dropwise to a solution of 3.4 g of piperidine obtained in stage 1 of Example 29 in CH₃CN (20 ml), and the mixture is stirred at AT for 18 hours. The mixture is concentrated to dryness and the resulting product is chromatographed (Al₂O₃), elution being carried out with DCM/EtOAc (v/v; 1/1), and 3 g of expected product are obtained.

Stage 2: [2-(tert-Butoxycarbonylpiperidin-4-ylamino)-ethyl]phosphonic acid diethyl ester

A mixture of compound obtained in stage 1 (3 g) and palladium hydroxide on charcoal is brought to reflux in EtOH (25 ml). After refluxing for 2 h 30, filtration and concentration are carried out so as to obtain 2.2 g of expected compound.

Stage 3: [2-(1-{4-[4-(3,4-Difluorophenylamino)pyrimidin-2-ylamino]benzenesulphonyl}piperidin-4-ylamino)ethyl]phosphonic acid diethyl ester

According to the protocol described in stage 1 of Example 2, 800 mg of difluoro derivative of procedure 2b and 880 mg of compound obtained in stage 2 make it possible to obtain 1.3 g of an intermediate compound which is subjected to a decarboxylation reaction to give 1 g of the expected compound.

MH+=625.0

Mp=149° C.

NMR (1H, DMSO)

1.25 (t, 6); 1.65 (m, 2); 2.09 (m, 2); 2.14-2.39 (unresolved peak, 4); 2.91-3.21 (2m, 3); 3.74 (m, 2); 4.05 (m, 4); 6.53 (d, 1); 7.35 (m, 1); 7.44 (m, 1); 7.71 (d, 2); 7.87 (d, 2); 7.99 (m1); 8.13 (d, 1); 9.26 (ls, 2); 10.75 (ls, 2).

### Example 31

### [2-(1-{4-[4-(4-Fluorophenylamino)pyrimidin-2-ylamino]benzenesulphonyl}piperidin-4-ylamino)ethyl]phosphonic acid diethyl ester

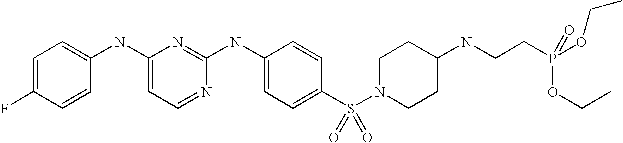

According to the protocol described in stages 3 and 4 of Example 32 using 800 mg of fluoro derivative of procedure 2a and 920 mg of the corresponding amine, 1 g of expected final compound is obtained.

MH+=607.1

Mp=195° C.

NMR (1H, DMSO)

1.25 (t, 6); 1.65 (m, 2); 2.09 (m, 2); 2.14-2.37 (unresolved peak, 4); 2.86-3.24 (2m, 3); 3.74 (d, 2); 4.02 (m, 4); 6.54 (d, 1); 7.26 (t, 2); 7.63 (m, 2); 7.68 (d, 2); 7.81 (d, 2); 8.09 (d, 1); 9.33 (ls, 2); 10.74-11.13 (2ls, 2).

### Example 32

### (1-{4-[4-(4-Fluoro-3-methylphenylamino)pyrimidin-2-ylamino]benzenesulphonyl}piperidin-4-yl)-(3-fluoropyridin-4-yl)methanol

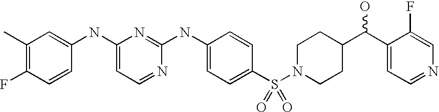

Stage 1: (3-Fluoropyridin-4-yl)piperidin-4-ylmethanol

According to Patent (WO/2005/059107), 12 ml of LDA (1.8 M) are added to a cold (−90° C.) solution of 1.82 g of 3-fluoropyridine in 50 ml of THF. The solution is stirred under nitrogen for 30 minutes while maintaining the same temperature. A solution of 2 g of 4-carboxaldehydepiperidine-1-carboxylic acid tert-butyl ester in 22 ml of THF is slowly added while maintaining the temperature below −70° C. The reaction mixture is stirred at this temperature for 30 minutes. The temperature is allowed to increase to −20° C. in 1 hour. 40 ml of a saturated solution of NH₄Cl are added slowly. After separation by settling out, the organic phase is washed with a 10% solution of Na₂CO₂, and then with a saturated solution of NaCl, and dried over MgSO₄, filtered and concentrated under vacuum. After silica chromatography, 1.82 g of an intermediate are obtained, which intermediate is subjected to a decarboxylation reaction to give 510 mg of 1-H-piperidine derivative.

Stage 2: (1-{4-[4-(4-Fluoro-3-methylphenylamino)pyrimidin-2-ylamino]benzenesulphonyl}piperidin-4-yl)-(3-fluoropyridin-4-yl)methanol

According to the protocol described in stage 1 of procedure 3a, using 600 mg of fluoro derivative of procedure 2c and 407 mg of compound obtained in stage 1, after the usual treatment, silica chromatography, elution being carried out with DCM/MeOH (98/2; v/v), and recrystallization from DCM-iPr₂O, 500 mg of expected product are obtained in the form of a mixture of two isomers.

MH+=570.2

Mp=142° C.

NMR (1H, DMSO)

1.26 (m, 1); 1.28-1.43 (unresolved peak, 2); 1.50 (unresolved peak, 1); 1.70 (d, 1); 2.10 (td, 2); 2.23 (s, 3); 3.62 (t, 2); 4.62 (t, 1); 5.63 (d, 1); 6.28 (d, 1); 7.10 (t, 1); 7.39-7.49 (m, 2); 7.53 (d, 2); 7.57 (dd, 1); 7.95 (d, 2); 8.07 (d, 1); 8.34 (d, 1); 8.45 (s, 1); 9.42 (s, 1); 9.69 (s, 1).

### Example 33

### (enantiomer 1): (1-{4-[4-(4-Fluoro-3-methylphenylamino)pyrimidin-2-ylamino]benzenesulphonyl}-piperidin-4-yl)-(3-fluoropyridin-4-yl)methanol

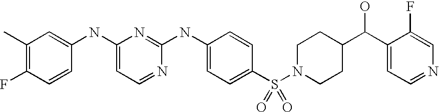

The separation of the two enantiomers of Example 32 is carried out by chiral chromatography (detection: UV 254 nm; stationary phase: chiralpakAD-10 μm 250×4.6 mm; phase mobile: 60% EtOH-40% heptane; flow rate: 1 ml/min). During this separation, 99.8 mg of the first enantiomer are obtained.

Tr=8.47 min

MH+=570.2

### Example 34

### (enantiomer 2): (1-{4-[4-(4-Fluoro-3-methylphenylamino)pyrimidin-2-ylamino]benzenesulphonyl}-piperidin-4-yl)-(3-fluoropyridin-4-yl)methanol

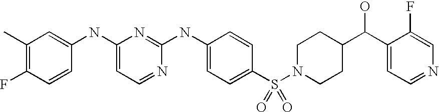

During the chiral chromatography step described in Example 33, 92.2 mg of the second enantiomer are obtained.

Tr=12.26 min.

MH+=570.2

### Example 35

### 1-(1-{4-[4-(4-Fluoro-3-methylphenylamino)pyrimidin-2-ylamino]benzenesulphonyl}piperidin-4-yl)-2-(3-methylpyridin-2-yl)ethanol (racemic)

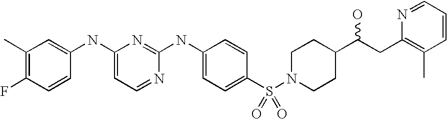

Stage 1: 2-(3-Methylpyridin-2-yl)-1-piperidin-4-ylethanol

According to the protocol described in stage 1 of Example 32, using 2 g of 2,3-dimethylpyridine and 2 g of 4-carboxaldehydepiperidine-1-carboxylic acid tert-butyl ester, 650 mg of expected 1-H-piperidine derivative are obtained.

Stage 2: 1-(1-{4-[4-(4-Fluoro-3-methylphenylamino)pyrimidin-2-ylamino]benzenesulphonyl}piperidin-4-yl)-2-(3-methylpyridin-2-yl)ethanol (racemic)

According to the protocol described in stage 2 of Example 32, using 350 mg of fluoro derivative of procedure 2c and 200 mg of piperidine obtained in stage 1, 200 mg of expected product are obtained in the form of a mixture of two isomers.

MH+=577.2

Mp=205-207° C.

1H NMR (DMSO):

1.25 (m, 1); 1.30 (unresolved peak, 2); 1.56 (m, 1); 1.67 (d, 1); 2.16 (m, 1); 2.23 (s, 3); 2.70 (s, 3); 3.61 (t, 2); 4.32 (t, 1); 5.45 (d, 1); 6.27 (d, 2); 7.08 (t, 1); 7.27 (d, 2); 7.45 (m, 1); 7.52 (d, 2); 7.58 (m, 1); 7.96 (d, 2); 8.02 (d, 1); 8.44 (d, 2); 9.47 (s, 1); 9.62 (s, 1).

### Example 36

### (1-{4-[4-(4-Fluoro-3-methylphenylamino)pyrimidin-2-ylamino]benzenesulphonyl}piperidin-4-yl)-pyridin-4-ylmethanol (racemic)

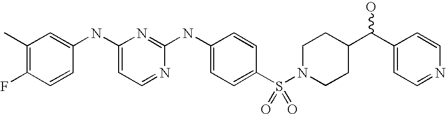

Stage 1: Piperidin-4-ylpyridin-4-ylmethanol

According to the protocol described in stage 1 of Example 32, using 2 g of 3-bromopyridine and 2 g of 4-carboxaldehydepiperidine-1-carboxylic acid tert-butyl ester, 650 mg of a product are obtained, which product is subjected to hydrogenolysis at 3 bar (WO/2005/059107) in order to remove the bromine, and decarboxylation to give 220 mg of expected 1-H-piperidine derivative.

Stage 2: (1-{4-[4-(4-Fluoro-3-methylphenylamino)pyrimidin-2-ylamino]benzenesulphonyl}piperidin-4-yl)-pyridin-4-ylmethanol (racemic)

According to the protocol described in stage 2 of Example 32, using 355 mg of fluoro derivative of procedure 2c and 174 mg of piperidine obtained in stage 1, 200 mg of expected product are obtained in the form of a mixture of two isomers.

MH+=549.4

Mp=162° C.

1H NMR (DMSO):

1.27 (m, 1); 1.35 (unresolved peak, 2); 1.46 (m, 1); 1.65 (d, 1); 2.06 (m, 1); 2.23 (s, 3); 3.61 (t, 2); 4.32 (t, 1); 5.45 (d, 1); 6.27 (d, 2); 7.09 (t, 1); 7.25 (d, 2); 7.45 (m, 1); 7.52 (d, 2); 7.57 (m, 1); 7.94 (d, 2); 8.06 (d, 1); 8.46 (d, 2); 9.41 (s, 1); 9.68 (s, 1).

### Example 37

### (1-{4-[4-(4-Fluorophenylamino)pyrimidin-2-ylamino]benzenesulphonyl}piperidin-4-yl)-(3-fluoro-pyridin-4-yl)methanol (racemic)

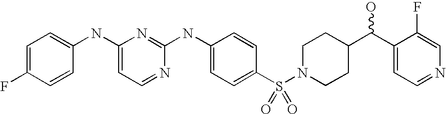

According to the protocol described in stage 2 of Example 32, using 400 mg of fluoro derivative of procedure 2a and 270 mg of piperidine obtained in stage 1 of Example 32, 115 mg of expected product are obtained in the form of a mixture of two isomers.

MH+=553.2

Mp=140° C.

NMR (1H, DMSO)

1.18-1.43 (unresolved peak, 3); 1.49 (m, 1); 1.70 (d, 1); 2.10 (t, 2); 3.62 (t, 2); 4.62 (t, 1); 5.63 (d, 1); 6.29 (d, 1); 7.17 (t, 2); 7.44 (t, 1); 7.55 (d, 2); 7.70 (dd, 2); 7.96 (d, 2); 8.09 (d, 1); 8.40 (d, 1); 8.46 (d, 1); 9.50 (s, 1); 9.71 (s, 1).

### Example 38

### 1-(1-{4-[4-(4-Fluoro-3-methylphenylamino)pyrimidin-2-ylamino]benzenesulphonyl}piperidin-4-yl)-2-(4-methylpyridin-2-yl)ethanol (racemic)

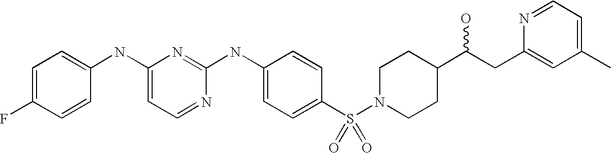

Stage 1: 2-(4-Methylpyridin-2-yl)-1-piperidin-4-ylethanol

According to the protocol described in stage 1 of Example 32, using 2 g of 2,4-dimethylpyridine and 2 g of 4-carboxaldehydepiperidine-1-carboxylic acid tert-butyl ester, 680 mg of expected 1-H-piperidine derivative are obtained.

Stage 2: 1-(1-{4-[4-(4-Fluoro-3-methylphenylamino)pyrimidin-2-ylamino]benzenesulphonyl}piperidin-4-yl)-2-(3-methylpyridin-2-yl)ethanol (racemic)

According to the protocol described in stage 2 of Example 32, using 500 mg of fluoro derivative of procedure 2c and 300 mg of piperidine obtained in stage 1, 270 mg of expected product are obtained in the form of a mixture of two isomers.

MH+=563.1

Mp=103.2-104.5° C.

NMR (1H, DMSO)

1H NMR (DMSO):

1.23 (m, 1); 1.28 (unresolved peak, 2); 1.50 (m, 1); 1.70 (d, 1); 2.12 (m, 1); 2.29 (s, 3); 2.68 (s, 3); 3.63 (t, 2); 4.30 (t, 1); 5.39 (d, 1); 6.20 (d, 2); 7.13 (t, 1); 7.24 (d, 2); 7.40 (m, 1); 7.58 (d, 2); 7.63 (m, 1); 7.98 (d, 2); 8.08 (d, 1); 8.40 (d, 2); 9.49 (s, 1); 9.67 (s, 1).

### Example 39

### {4-[4-(4-Fluoro-3-methylphenylamino)pyrimidin-2-ylamino]phenyl}(piperidin-4-yl)-(3-fluoro-pyridin-4-yl)methanol)methanone (racemic)

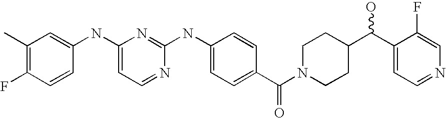

According to the protocol described in stage 1 of procedure 4a, using 320 mg of acid obtained in procedure 2c and piperidine obtained in stage 1 of Example 32, 290 mg of expected product are obtained in the form of a mixture of two isomers.

MH+=531.2

Mp=115° C. (formation of a foam)

NMR (1H, DMSO)

1.08-1.98 (unresolved peak, 5); 2.23 (d, 3); 2.80 (sl, 2); 4.70 (t, 1); 5.65 (d, 1); 6.22 (d, 1); 7.09 (s, 1); 7.26 (d, 2); 7.377.66 (unresolved peak, 3); 7.81 (d, 2); 8.04 (d, 1); 8.44 (d, 1); 8.50 (d, 1); 9.33 (s, 1); 9.38 (s, 1).

### Example 40

### [4-R-(Aminophenylmethyl)piperidin-1-yl]-{4-[4-(4-fluorophenylamino)pyrimidin-2-ylamino]phenyl}methanone

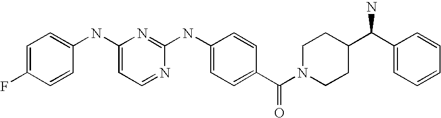

DIPEA (1.5 ml), BOP (360 mg), and then, in small portions in 30 minutes, 300 mg of acid obtained in procedure 2a are added, at ambient temperature, in this order, to a solution containing 220 mg of the commercially available amine phenylpiperidin-4-R-ylmethylamine in 20 ml of a DCM/DMF mixture (v/v; 1/1). The mixture is stirred overnight. The mixture is evaporated to dryness, carbonated (K₂CO₃), water is added and the mixture is extracted with EtOAc. After treatment, chromatography (SiO₂) is performed, elution being carried out with DCM/MeOH (v/v; 94/6), and recrystallization is carried out from DCM/iPr₂O.

MH+=497.2

Mp=130-185° C.

[α]D=+40 (c=0.15, MeOH)

NMR (1H, DMSO)

1.07-1.41 (unresolved peak, 3); 1.74 (m, 1); 1.88 (d, 2); 2.41 (sl, 1); 2.82 (m, 2); 3.68 (d, 1); 4.09 (dl, 2); 6.23 (d, 1); 7.13 (t, 2); 7.17-7.38 (unresolved peak, 7); 7.66 (m, 2); 7.76 (d, 2); 8.03 (d, 1); 9.11 (s, 1); 9.20 (s, 1).

### Example 41

### [4-S-(Aminophenylmethyl)piperidin-1-yl]-{4-[4-(4-fluorophenylamino)pyrimidin-2-ylamino]phenyl}methanone

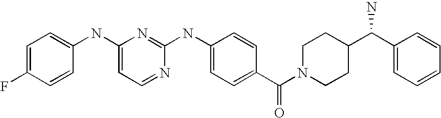

According to the protocol described in Example 40, using the amine in the S configuration and the acid obtained in procedure 2a, the expected product is obtained.

MH+=497.2

Mp=130-185° C.

[α]D=−34 (c=0.117, MeOH)

NMR (1H, DMSO)

1.07-1.41 (unresolved peak, 3); 1.75 (m, 1); 1.88 (d, 1); 2.82 (m, 2); 3.68 (d, 1); 4.09 (dl, 2): 6.23 (d, 1); 7.13 (t, 2); 7.17-7.38 (unresolved peak, 7); 7.66 (m, 2); 7.76 (d, 2); 8.03 (d, 1); 9.11 (s, 1); 9.20 (s, 1).

### Example 42

### N*4*-(4-Fluoro-3-methylphenyl)-N*2*-{4-[3-(pyridin-3-yloxy)piperidine-1-sulphonyl]phenyl}-pyrimidine-2,4-diamine (racemic)

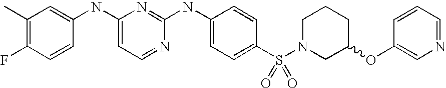

DIPEA (1 ml) and then the sulphonyl chloride obtained in procedure 1c (1.4 g) are added to a suspension of 800 mg of racemic 3-pyridyloxypiperidine dihydrochloride (obtained according to the synthesis described in J. Med. Chem. 43, 11, 2000, 2217-2226) in 20 ml of DCM, and the mixture is stirred overnight. The mixture is evaporated to dryness, carbonated (K₂CO₃) water is added, and the mixture is extracted with EtOAc. After treatment, chromatography (SiO₂) is performed, elution being carried out with DCM/MeOH (v/v; 97/3), and recrystallization is carried out from DCM/iPr₂O so as to obtain the expected product.

MH+=535.1

Mp=113° C.

NMR (1H, DMSO)

1.42-1.96 (unresolved peak, 4); 2.24 (s, 3); 2.76-3.01 (unresolved peak, 3); 3.24 (d, 2); 4.61 (m, 1); 6.29 (d, 1); 7.10 (t, 1); 7.29-7.65 (unresolved peak, 6); 7.98 (d, 2); 8.07 (d, 1); 8.19 (d, 1); 8.30 (d, 1); 9.42 (sl, 1); 9.71 (sl, 1).

### Example 43

### {4-R-[Amino-(4-fluorophenyl)methyl]piperidin-1-yl}-{4-[4-(4-fluorophenylamino)pyrimidin-2-ylamino]phenyl}methanone

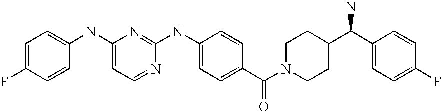

According to the protocol described in Example 40, using the amine R and the acid obtained in procedure 2a, the expected product is obtained.

MH+=515.2

Mp=184-185° C.

[α]D=+49 (c=0.103, MeOH)

NMR (1H, DMSO)

0.98-1.38 (unresolved peak, 3); 1.64 (m, 1); 1.87 (sl, 3); 2.77 (sl, 2); 3.60 (d, 1); 3.63-4.81 (unresolved peak, 2); 6.22 (d, 1); 7.03-7.20 (unresolved peak, 4); 7.24 (d, 2); 7.34 (m, 2); 7.70 (m, 2); 7.77 (d, 2); 8.04 (d, 1); 9.38 (s, 1); 9.42 (s,

### Example 44

### {4-S-[Amino-(4-fluorophenyl)methyl]piperidin-1-yl}-{4-[4-(4-fluorophenylamino)pyrimidin-2-ylamino]phenyl}methanone

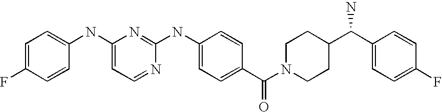

According to the protocol described in Example 40, using the amine S and the acid obtained in procedure 2a, the expected product is obtained.

MH+=515

Mp=182-184° C.

[α]D=−47 (c=0.127, MeOH)

NMR (1H, DMSO)

0.99-2.15 (unresolved peak, 7); 2.72 (broad multiplet, 2); 3.54 (multiplet, 1); 3.61-4.97 (broad signal, 2); 6.18 (d, 1); 6.97-7.41 (unresolved peak, 8); 7.57-7.82 (unresolved peak, 4); 7.99 (d, 1); 9.28-9.43 (2s, 2).

### Example 45

### (1-{4-[4-(4-Fluorophenylamino)pyrimidin-2-ylamino]benzenesulphonyl}-3-pyridin-3-ylmethylpiperidin-3-yl)methanol (racemic)

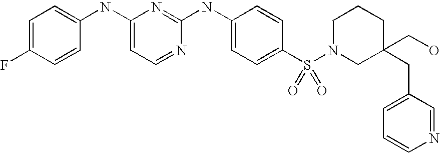

A solution of 500 mg of compound obtained in Example 14 in 10 ml of THF is added dropwise to a suspension of 30 mg of LiAlH₄ in 20 ml of THF and the reaction medium is left to stir for 18 h. The excess LiAlH₄ is destroyed by adding 2 ml of water followed by a few drops of concentrated sodium hydroxide. After filtration, the filtrate is concentrated under vacuum and the reaction crude is purified by silica chromatography (eluent: DCM/MeOH; 95/5). 210 mg of desired alcohol are obtained.

MH+=563.2

Mp=218° C.

NMR (1H, DMSO)

1.12 (sl, 2); 1.45-1.90 (2sl, 2); 2.23 (dl, 3); 2.46-3.15 (unresolved peak, 8); 4.81 (t, 1); 6.29 (d, 1); 7.12 (t, 1); 7.31 (m, 1); 7.47 (m, 1); 7.53-7.70 (unresolved peak, 4); 8.00 (d, 2); 8.08 (d, 1); 8.38-8.48 (unresolved peak, 2); 9.43 (s, 1); 9.73 (s, 1).

### Example 46

### {4-[4-(4-Fluoro-3-methylphenylamino)pyrimidin-2-ylamino]phenyl}-(3-pyridyloxypiperidin-1-yl)methanone (racemic)

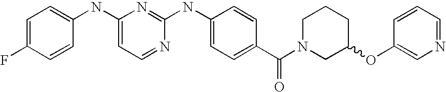

According to the protocol described in Example 40, using 350 mg of the 3-pyridyloxypiperidin-1-yl used in Example 42 and 580 mg of the acid obtained in procedure 2a, 228 mg of expected product are obtained.

MH+=485.0

Mp=120° C.

NMR (1H, DMSO)

1.42-2.14 (unresolved peak, 4); 3.37-4.23 (unresolved peak, 4); 4.57 (m, 1); 6.23 (d, 1); 7.15 (t, 2); 7.20-8.43 (unresolved peak, 11); 9.38 (s, 1); 9.43 (s, 1).

### Example 47

### 4-Pyrrolidin-1-ylmethyl-1-{4-[4-(4-fluoro-3-methylphenylamino)pyrimidin-2-ylamino]benzenesulphonyl}-piperidin-4-ol

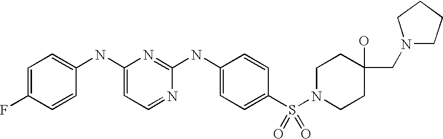

Stage 1: A solution of 4.5 g of sodium hydroxide in 48 ml of water is added dropwise to a solution containing 18.23 g of dimethyloxosulphonium in methylide and 0.485 g of tetrabutylammonium in 150 ml of toluene and the reaction medium is left to stir at 80° C. for 3 hours. After cooling, the mixture is washed with water, followed by drying over Na₂SO₄ and concentrated to dryness. 13 g of epoxide are thus obtained.

Stage 2: 1.5 g of the epoxide obtained in stage 1 is heated, in a sealed tube, at 80° C. for 4 hours in the presence of 1 g of pyrrolidine in 25 ml of ethanol. After the usual treatment, 1.5 g of amino alcohol are obtained, which are subjected to a decarboxylation reaction so as to give the expected piperidine-4-methylpyrrolidine.

Stage 3: According to the protocol described in stage 1 of procedure 3a, using 500 mg of sulphonyl chloride hydrochloride of procedure 1a and 370 mg of piperidine obtained in stage 2, 140 mg of expected sulphonamide are obtained.

MH+=526.9

Melting point=148° C.

### Example 48

### {4-[4-(4-Fluoro-3-methylphenylamino)pyrimidin-2-ylamino]phenyl}-(4-pyrrolidin-1-ylmethylpiperidin-4-ol)methanone

According to the protocol described in Example 40, using 428 mg of the piperidine-4-methylpyrrolidine obtained in stage 2 of Example 47 and 500 mg of the acid obtained in procedure 2a, 280 mg of expected product are obtained.

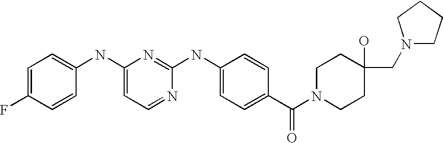

MH+=491.1

Melting point=204° C.

### Example 49

### N²-4-[(3-{[bis(1H-Pyrazol-4-ylmethyl)amino]-methyl}piperidin-1-yl)sulphonyl]phenyl}-N⁴-(4-methylphenyl)pyrimidine-2,4-diamine

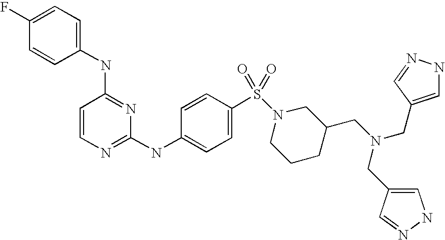

In the same manner as in Example 1, using 500 mg of the compound of procedure 3b and 138 mg of 1-methyl-1H-pyrrole-2-carbaldehyde, 150 mg of expected product are obtained.

MH+=617.1

1H NMR (DMSO):

1.18 (m, 1); 1.24-2.88 (unresolved peak, 6); 2.72 (m, 2); 3.27-3.54 (2d, 2); 4.11 (t, 2); 4.25 (d, 2); 6.45 (d, 1); 2.08 (t, 2); 7.46-7.76 (unresolved peak, 6); 7.83 (s, 4); 8.05 (d, 1).

### Examples 50 to 78

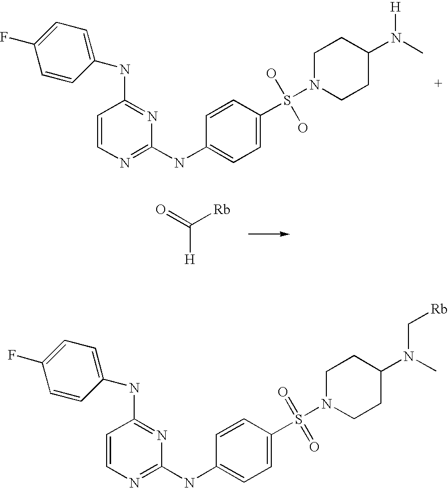

In the same manner as in Example 1, reaction of the acid of procedure 4a with commercially available aldehydes (or ketones), the following products (30 examples in the table below which constitute Examples 50 to 78 of the present invention) are obtained by adapting the procedure according to the protocol below.

A solution of 0.12 mmol of aldehyde in 1.0 ml of THF and 0.3 ml of AcOH is added to 0.10 mmol of the product of procedure 4a in 2.0 ml of THF. Finally, 128 mg of polymer bearing CNBH₃ are added and the mixture is left to stir under an argon atmosphere overnight at AT. The reaction mixture is filtered, the filtrate is washed with 5 ml of THF and the resulting product is concentrated under vacuum. The reaction crude is dissolved in 2 ml of DMF and purified by preparative HPLC so as to give the expected product described in the form of a trifluoroacetic acid salt.

| ex | STRUCTURE | MH+ | NAME |
|---|---|---|---|
| 50 | 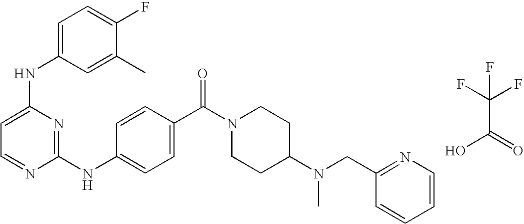 | 526.29 | {4-[4-(4-fluoro- 3-methylphenyl- amino)pyrimidin- 2-ylamino]- phenyl}[4- (methylpyridin- 2-ylmethyl- amino)piperidin- 1-yl]methanone; |
| 51 | 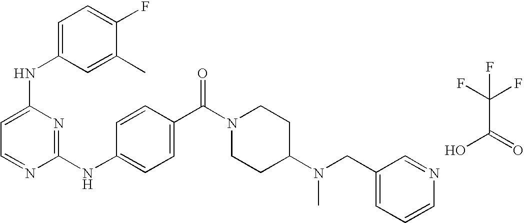 | 526.30 | {4-[4-(4-fluoro- 3-methylphenyl- amino)pyrimidin- 2-ylamino]- phenyl}-[4- (methylpyridin- 3-ylmethyl- amino)piperidin- 1-yl]methanone; |
| 52 | 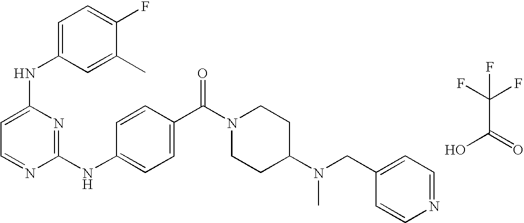 | 526.42 | {4-[4-(4-fluoro- 3-methylphenyl- amino)pyrimidin- 2-ylamino]- phenyl}-[4- (methylpyridin- 4-ylmethyl- amino)piperidin- 1-yl]methanone; |
| 53 | 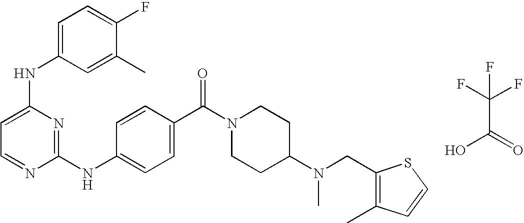 | 545.27 | {4-[4-(4-fluoro- 3-methylphenyl- amino)pyrimidin- 2-ylamino]- phenyl}-{4- [methyl-(3- methylthiophen- 2-ylmethyl)- aminol]piperidin- 1-yl}methanone |
| 54 | 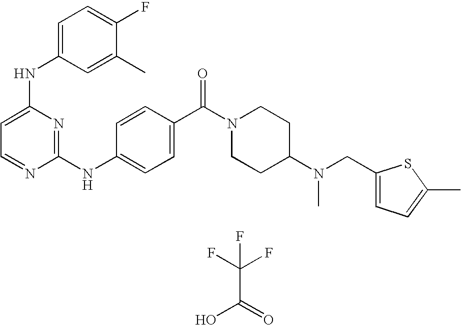 | 545.27 | {4-[4-(4-fluoro- 3-methylphenyl- amino)pyrimidin- 2-ylamino]- phenyl}-{4- [methyl-(5- methylthiophen- 2-ylmethyl)- aminol]piperidin- 1-yl}methanone |
| 55 | 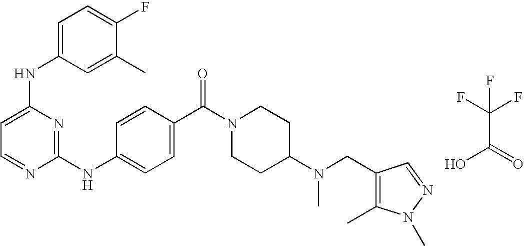 | 543.32 | {4-[(1,5- dimethyl-1H- pyrazol-4- ylmethyl)- methylamino]piper- idin-1-yl}-{4- [4-(4-fluoro-3- methylphenyl- amino)pyrimidin-2- ylamino]phenyl} methanone |
| 56 | 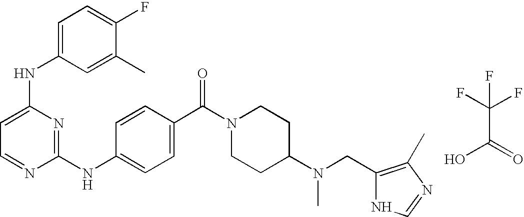 | 529.41 | {4-[4-(4-fluoro- 3-methylphenyl- amino)pyrimidin- 2-ylamino]- phenyl}-{4- [methyl-(5- methyl-3H- imidazol-4- ylmethyl)amino]- piperidin-1- yl}methanone; |
| 57 | 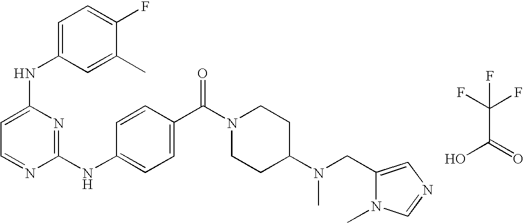 | 529.41 | {4-[4-(4-fluoro- 3-methylphenyl- amino)pyrimidin- 2-ylamino]- phenyl}-{4- [methyl-(3- methyl-3H- imidazol-4- ylmethyl)amino]- piperidin-1- yl}methanone; |
| 58 | 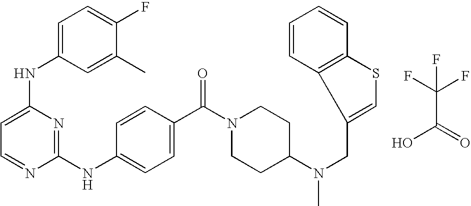 | 581.28 | [4-(benzo[b]- thiophen-3-yl- methyl- methylamino)- piperidin-1-yl]- {4-[4-(4-fluoro- 3-methyl- phenylamino)- pyrimidin-2- ylamino]phenyl}- methanone; |
| 59 | 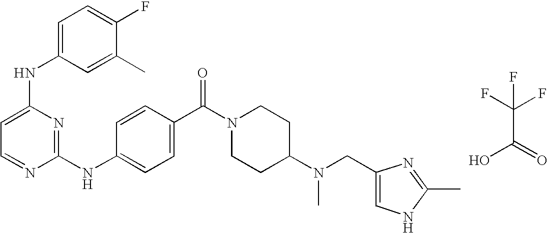 | 529.42 | {4-[4-(4-fluoro- 3-methylphenyl- amino)pyrimidin- 2-ylamino]- phenyl}-{4- [methyl-(2- methyl-1H- imidazol-4- ylmethyl)amino]- piperidin-1- yl}methanone; |
| 60 | 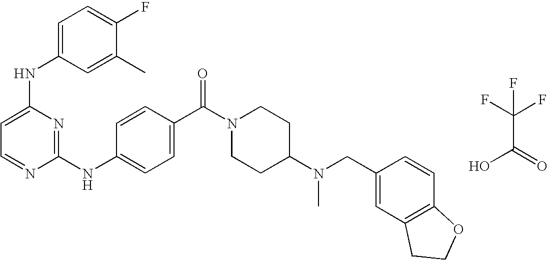 | 567.32 | {4-[(2,3- dihydro- benzofuran-5- ylmethyl)- methylamino]- piperidin-1-yl}- {4-[4-(4-fluoro- 3-methylphenyl- amino)pyrimidin- 2-ylamino]- phenyl}methanone |
| 61 | 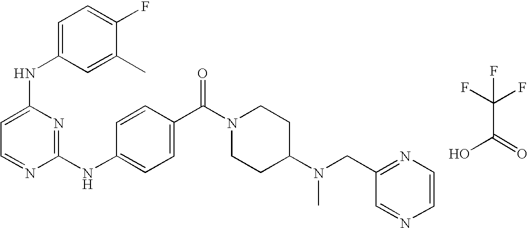 | 527.29 | {4-[4-(4-fluoro- 3-methylphenyl- amino)pyrimidin- 2-ylamino]- phenyl}-[4- (methylpyrazin- 2-ylmethyl- amino)piperidin- 1-yl]methanone; |
| 62 | 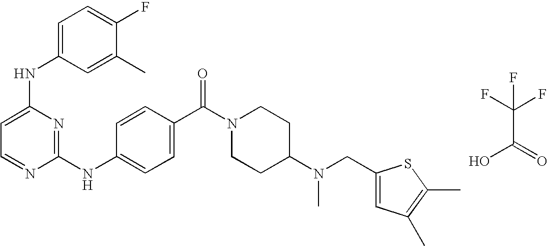 | 559.28 | {4-[(4,5- dimethylthiophen- 2-ylmethyl)- methylamino]- piperidin-1-yl}- {4-[4-(4-fluoro- 3-methylphenyl- amino)pyrimidin- 2-ylamino]- phenyl}methanone |
| 63 | 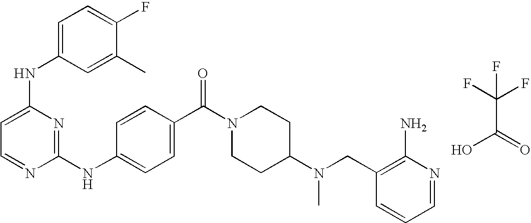 | 541.45 | {4-[(2- aminopyridin-3- ylmethyl)- methylamino]piper- idin-1-yl}-{4- [4-(4-fluoro-3- methylphenyl- amino)pyrimidin-2- ylamino]phenyl} methanone |
| 64 | 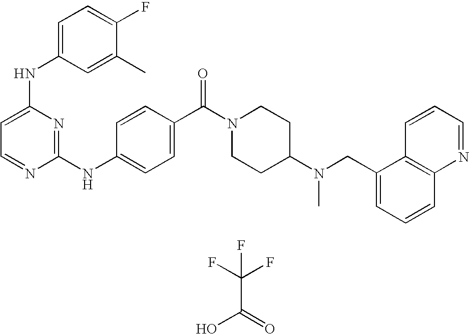 | 576.31 | {4-[4-(4-fluoro- 3-methylphenyl- amino)pyrimidin- 2-ylamino]- phenyl}-[4- (methylquinolin- 5- ylmethylamino)- piperidin-1- yl]methanone; |
| 65 | 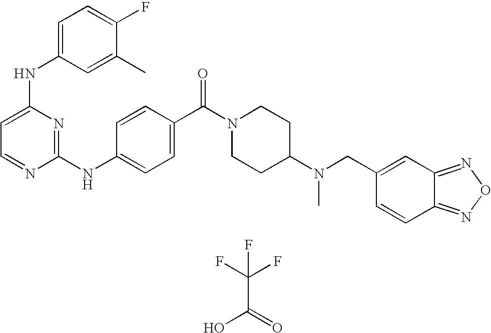 | 567.29 | [4-(benzo- [1,2,5]oxadiazol- 5-ylmethyl- methylamino)- piperidin-1-yl]- {4-[4-(4-fluoro- 3-methylphenyl- amino)pyrimidin- 2- ylamino]phenyl}- methanone; |
| 66 | 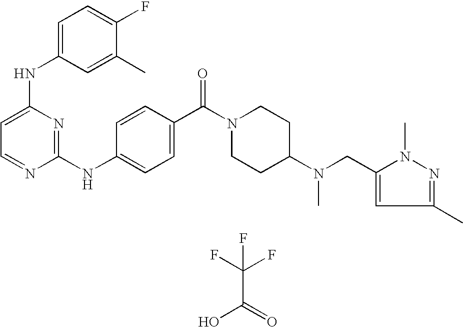 | 543.31 | {4-[(2,5- dimethyl-2H- pyrazol-3- ylmethyl)- methylamino]- piperidin-1-yl}- {4-[4-(4-fluoro- 3-methylphenyl- amino)pyrimidin- 2-ylamino]- phenyl}methanone |
| 67 | 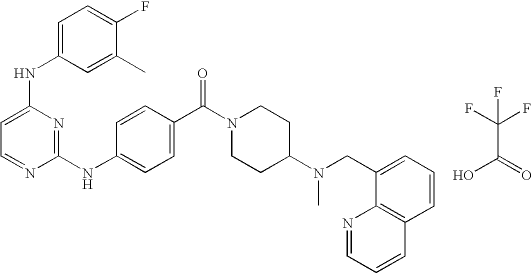 | 576.29 | {4-[4-(4-fluoro- 3-methylphenyl- amino)pyrimidin- 2-ylamino]- phenyl}-[4- (methylquinolin- 8-ylmethyl- amino)piperidin- 1-yl]methanone |
| 68 | 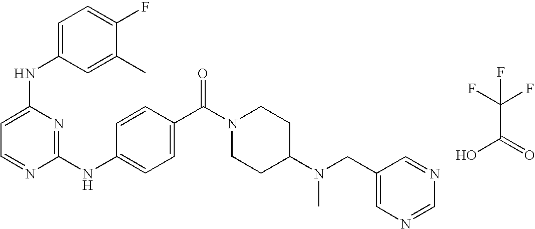 | 527.29 | {4-[4-(4-fluoro- 3-methyl- phenylamino)- pyrimidin-2- ylamino]phenyl}- [4-(methyl- pyrimidin-5- ylmethylamino)- piperidin-1- yl]methanone; |
| 69 | 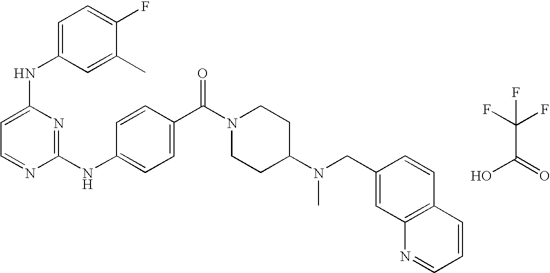 | 576.31 | {4-[4-(4-fluoro- 3-methylphenyl- amino)pyrimidin- 2-ylamino]- phenyl}-[4- (methylquinolin- 7-ylmethyl- amino)piperidin- 1-yl]methanone; |
| 70 | 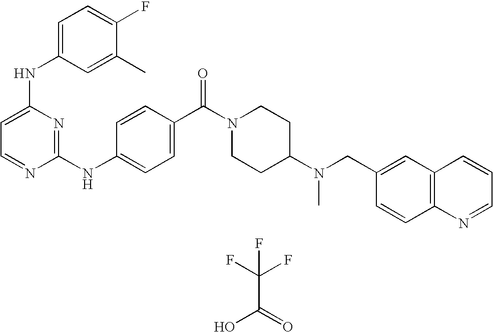 | 576.31 | {4-[4-(4-fluoro- 3-methylphenyl- amino)pyrimidin- 2-ylamino]- phenyl}-[4- (methylquinolin- 6-ylmethyl- amino)piperidin- 1-yl]methanone; |
| 71 | 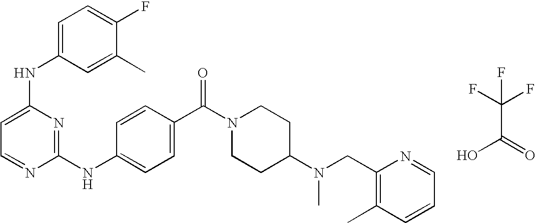 | 540.29 | {4-[4-(4-fluoro- 3-methylphenyl- amino)pyrimidin- 2-ylamino]- phenyl}-{4- [methyl-(3- methylpyridin-2- ylmethyl)- aminol]piperidin- 1-yl}methanone |
| 72 | 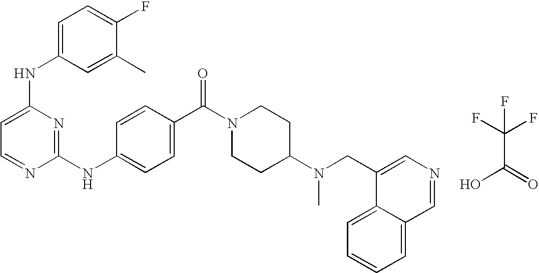 | 576.44 | {4-[4-(4-fluoro- 3-methylphenyl- amino)pyrimidin- 2-ylamino]- phenyl}-[4- (isoquinolin-4- ylmethyl- methylamino)- piperidin-1- yl]methanone |
| 73 | 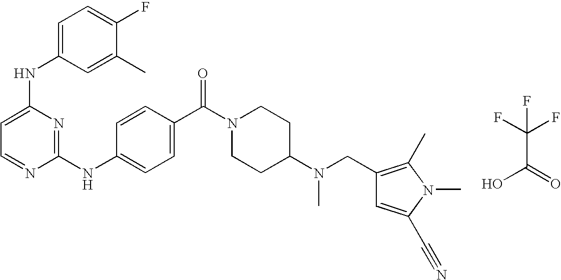 | 567.31 | 4-{[(1-{4-[4-(4- fluoro-3- methylphenyl- amino)pyrimidin- 2-ylamino]- benzoyl}- piperidin-4- yl)methylamino]- methyl}-1,5- dimethyl-1H- pyrrole-2- carbonitrile |
| 74 | 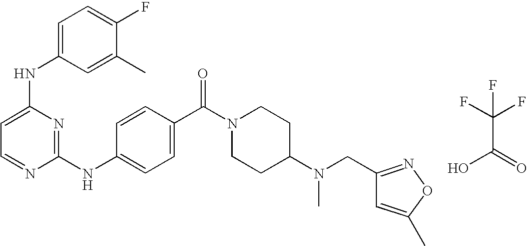 | 530.27 | {4-[4-(4-fluoro- 3-methylphenyl- amino)pyrimidin- 2-ylamino]- phenyl}-{4- [methyl-(5- methylisoxazol- 3-ylmethyl)- amino]piperidin- 1-yl}methanone |
| 75 | 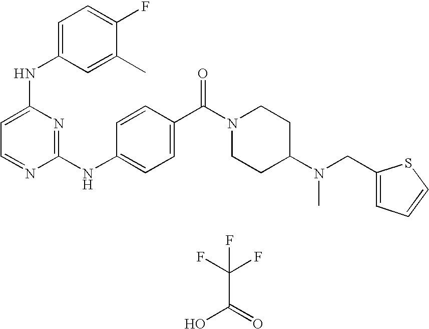 | 531.24 | {4-[4-(4-fluoro- 3-methylphenyl- amino)pyrimidin- 2-ylamino]- phenyl}-[4- (methylthiophen- 2-ylmethyl- amino)piperidin- 1-yl]methanone |
| 76 | 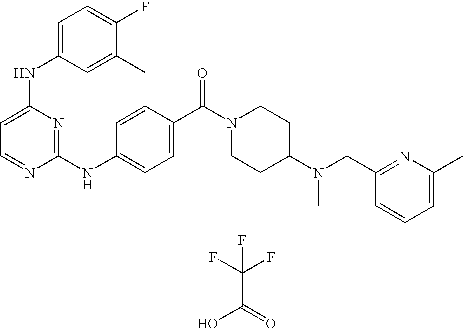 | 540.28 | {4-[4-(4-fluoro- 3-methylphenyl- amino)pyrimidin- 2-ylamino]- phenyl}-{4- [methyl-(6- methylpyridin-2- ylmethyl)amino]- piperidin-1- yl}methanone |
| 77 | 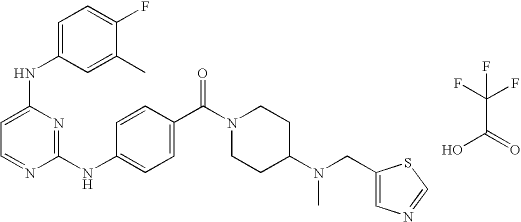 | 532.24 | {4-[4-(4-fluoro- 3-methylphenyl- amino)pyrimidin- 2-ylamino]- phenyl}-[4- (methyl-thiazol- 5-ylmethyl- amino)piperidin- 1-yl]methanone |
| 78 | 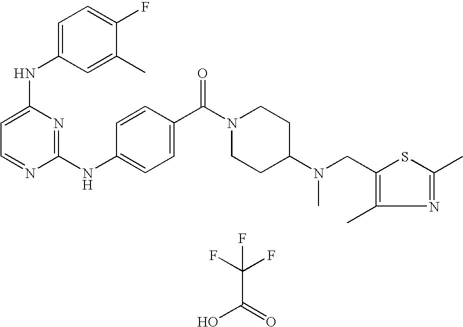 | 560.27 | {4-[(2,4- dimethylthiazol- 5-ylmethyl)- methylamino]- piperidin-1-yl}- {4-[4-(4-fluoro- 3-methylphenyl- amino)pyrimidin- 2-ylamino]- phenyl}methanone |

Purification Methods

Description of Preparative HPLC

Description of the GILSON instrument used:

Two 306 pumps with 100SC pump head.

One 806 pulse dampener.

One 811C mixer with 25 ml mixing chamber.

One 231XL injector+racks 21 and Rheodyne 7000L injection valve (5 ml stainless steel loop).

1 module 401 with 10 ml syringe.

1 injection valve actuator 819 with Rheodyne 7000L valve used in column selector.

One 215 fraction collector equipped with five 207 racks and 3-way valve for collection.

1 UV/visible detector 118.

One 506C interface.

Instrument controlled by the GILSON 2.0 software; collection is carried out as a function of the absorption of the UV detector.

LC columns of the type VP NUCLEODUR GRAVITY 100-10 C18 supplied by the company MACHEREY-NAGEL.

Basic HCOONH₄ (0.01M) aqueous NH₃ pH9-10

Solvents used:
“milli-Q” water 0.01M HCOONH₄ NH₄OH pH 9-10.
Acetonitrile for HPLC gradient type CHROMANORM Prolabo acid (0.07% TFA)

Solvents used:
“milli-Q” water containing 0.07% of TFA.
Acetonitrile containing 0.07% of TFA supplied by the company SD

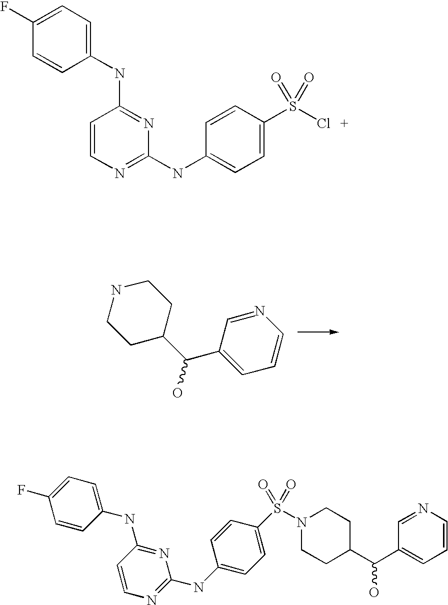

### Example 79

330 mg of the sulphonyl chloride of procedure 1a is suspended in 40 ml of CH₂Cl₂. 186 mg of commercially available amino alcohol (interchim BG206) are added, followed by 0.55 ml of TEA, and then the mixture is left to stir at ambient temperature overnight.

The reaction medium is evaporated in a rotary evaporator, then the residue is taken up with H₂O: 100 cm³ and the resulting product is extracted with 3×100 cm³ of EtOAc; the EtOAc phases are combined and evaporated in a rotary evaporator. The resulting product is purified by prep C18 HPLC, the MeCN is evaporated off, and the resulting product is freeze-dried. 152 mg of white freeze-dried material are obtained.

NMR: Comp>95% Mass: Comp>95%

With the various sulphonyl chlorides of procedures 1a, 1b or 1c, in the presence of the corresponding amines, the following compounds are obtained:

| Ex | STRUCTURE | MH+ | NAME |
|---|---|---|---|
| 79 | 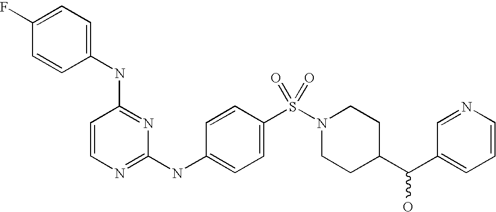 | 535 | (1-{[4-({4-[(4- fluorophenyl)amino]pyrimidin- 2-yl}amino)phenyl]sulphonyl}- piperidin-4-yl)(pyridin-3- yl)methanol |
| 80 | 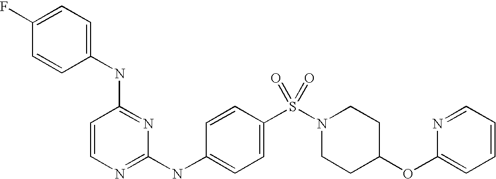 | 521 | N~4~-(4-fluorophenyl)-N~2~- (4-{[4-(pyridin-2- yloxy)piperidin-1-yl]sulphonyl}- phenyl)-pyrimidine-2,4-diamine |
| 81 | 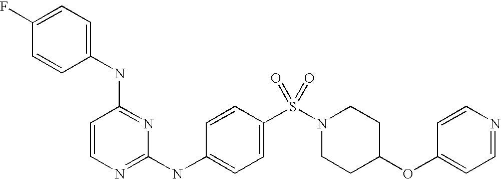 | 521 | N~4~-(4-fluorophenyl)-N~2~- (4-{[4-(pyridin-4- yloxy)piperidin-1-yl]sulphonyl}- phenyl)-pyrimidine-2,4-diamine |
| 82 | 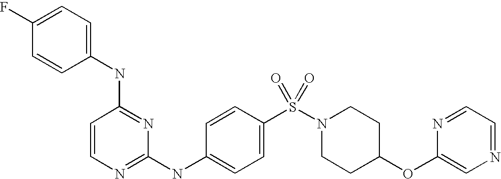 | 522 | N~4~-(4-fluorophenyl)-N~2~- (4-{[4-(pyrazin-2- yloxy)piperidin-1-yl]sulphonyl}- phenyl)-pyrimidine-2,4-diamine |
| 83 | 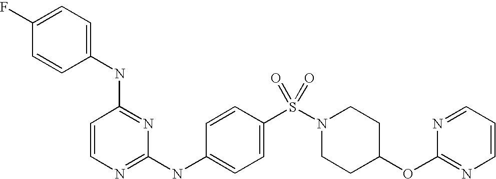 | 522 | N~4~-(4-fluorophenyl)-N~2~- (4-{[4-(pyrimidin-2- yloxy)piperidin-1-yl]sulphonyl}- phenyl)-pyrimidine-2,4-diamine |
| 84 | 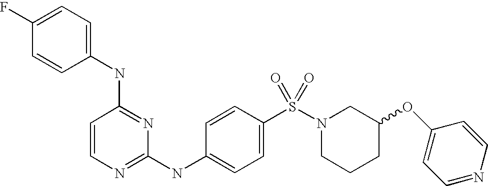 | 521 | N~4~-(4-fluorophenyl)-N~2~- (4-{[3-(pyridin-4- yloxy)piperidin-1-yl]sulphonyl}- phenyl)-pyrimidine-2,4-diamine |
| 85 | 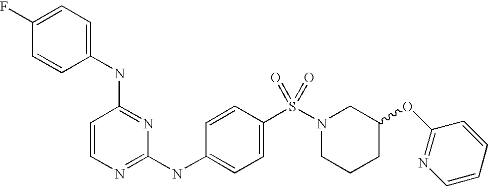 | 521 | N~4~-(4-fluorophenyl)-N~2~- (4-{[3-(pyridin-2- yloxy)piperidin-1-yl]sulphonyl}- phenyl)-pyrimidine-2,4-diamine |
| 86 | 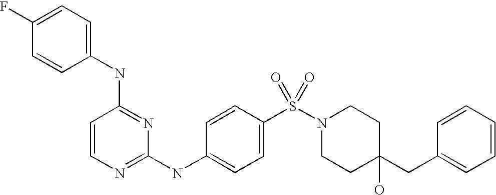 | 534 | 4-benzyl-1-{[4-({4-[(4- fluorophenyl)-amino]pyrimidin- 2-yl}amino)-phenyl]sulphonyl}- piperidin-4-ol |
| 87 | 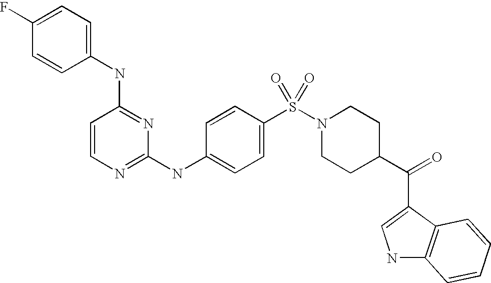 | 571 | (1-{[4-({4-[(4- fluorophenyl)amino]pyrimidin- 2-yl}amino)phenyl]sulphonyl}- piperidin-4-yl)(1H-indol-3- yl)methanone |
| 88 | 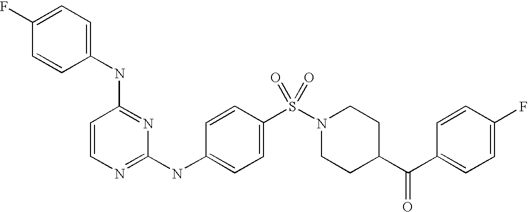 | 550 | (4-fluorophenyl)(1-{[4-({4-[(4- fluorophenyl)-amino]pyrimidin- 2-yl}amino)-phenyl]- sulphonyl}-piperidin-4- yl)methanone |
| 89 | 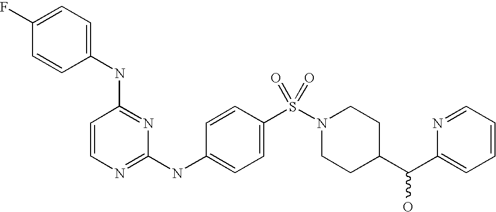 | 535 | (1-{[4-({4-[(4- fluorophenyl)amino]pyrimidin- 2-yl}amino)phenyl]sulphonyl}- piperidin-4-yl)(pyridin-2- yl)methanol |
| 90 | 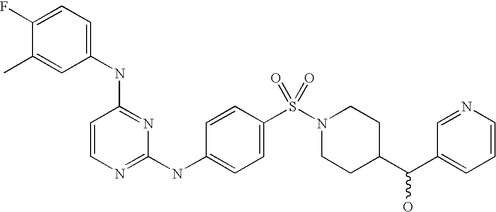 | 549 | (1-{[4-({4-[(4-fluoro-3- methylphenyl)-amino]pyrimidin- 2-yl}amino)-phenyl]- sulphonyl}-piperidin-4- yl)(pyridin-3-yl)methanol |
| 91 | 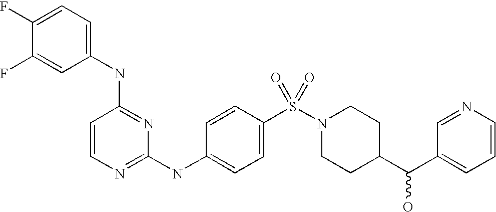 | 553 | (1-{[4-({4-[(3,4-difluorophenyl)- amino]pyrimidin-2-yl}amino)- phenyl]-sulphonyl}piperidin-4- yl)-(pyridin-3-yl)methanol |

The compounds obtained in Examples 79, 90 and 91 are separated by chiral chromatography, as in Example 32, so as to give respectively the following enantiomers (of undefined absolute configuration): Examples 92 & 93; Examples 94 & 95; Examples 96 & 97. The optical rotations are measured using DMSO as solvent. The concentrations are in mg/ml.

| Ex | STRUCTURE | αD | NAME |
|---|---|---|---|
| 92 | 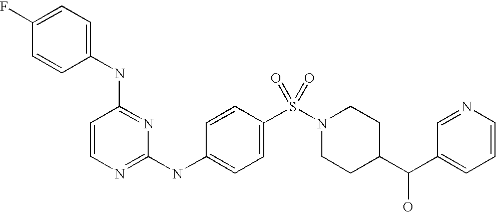 | −44.2 | enantiomer (1-{[4-({4-[(4- fluorophenyl)-amino]pyrimidin- 2-yl}amino)-phenyl]- sulphonyl}-piperidin-4- yl)(pyridin-2-yl)methanol |
| 93 | 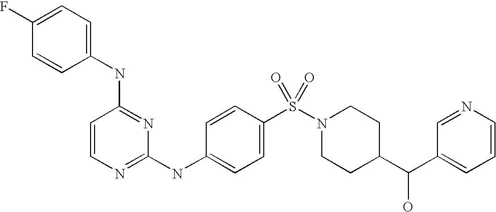 | 30.6 | enantiomer (1-{[4-({4-[(4- fluorophenyl)-amino]pyrimidin- 2-yl}amino)-phenyl]- sulphonyl}piperidin-4-yl)- (pyridin-2-yl)methanol |
| 94 | 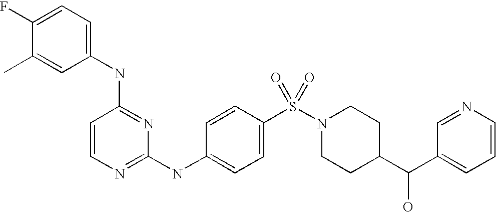 | 30.4 | enantiomer (1-{[4-({4-[(4- fluoro-3- methylphenyl)amino]pyrimidin- 2-yl}amino)phenyl]sulphonyl}- piperidin-4-yl)(pyridin-3- yl)methanol |
| 95 | 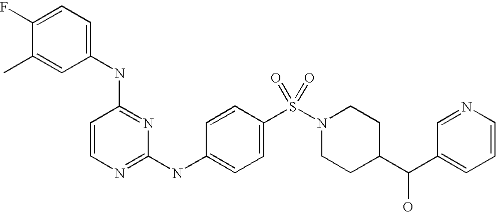 | −31.2 | enantiomer (1-{[4-({4-[(4- fluoro-3-methylphenyl)- amino]pyrimidin-2-yl}amino)- phenyl]-sulphonyl}piperidin-4- yl)-(pyridin-3-yl)methanol |
| 96 | 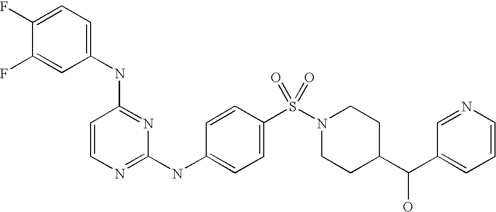 | 38.2 | enantiomer (1-{[4-({4-[(3,4- difluorophenyl)- amino]pyrimidin-2-yl}amino)- phenyl]-sulphonyl}piperidin-4- yl)-(pyridin-3-yl)methanol |
| 97 | 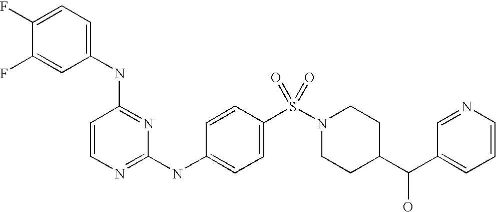 | −38.2 | enantiomer (1-{[4-({4-[(3,4- difluorophenyl)- amino]pyrimidin-2-yl}amino)- phenyl]-sulphonyl}piperidin-4- yl)-(pyridin-3-yl)methanol |

The compound of Example 88 (50 mg) is dissolved in 5 ml of methanol and 10 mg of sodium borohydride are added. After one hour, a further 3 mg of NaBH₄ are added and the reaction is left at ambient temperature for 2 h. Water is added, the mixture is then evaporated to dryness and the residue is purified by HLPC under basic conditions. 38 mg of white powder are obtained, which is the expected product (Example 98). In the same manner, Examples 99, 100, 101 are prepared by reduction of the corresponding ketones.

| Ex | STRUCTURE | MH+ | NAME |
|---|---|---|---|
| 98 | 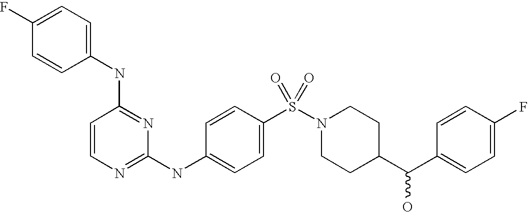 | 552 | (4-fluorophenyl) (1-{[4-({4- [(4-fluorophenyl)- amino]pyrimidin-2- yl}amino)-phenyl]- sulphonyl}-piperidin-4- yl)methanol |
| 99 | 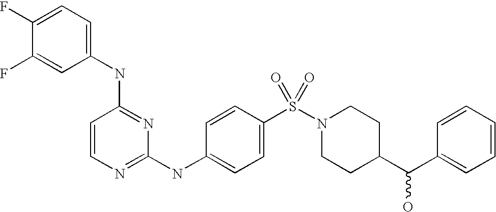 | 552 | (1-{[4-({4-[(3,4- difluorophenyl)- amino]pyrimidin-2- yl}amino)-phenyl]- sulphonyl}piperidin-4-yl)- (phenyl)methanol |
| 100 | 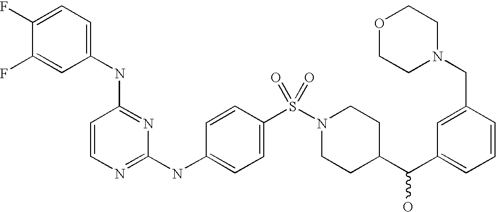 | 650 | (1-{[4-({4-[(3,4- difluorophenyl)- amino]pyrimidin-2- yl}amino)-phenyl]- sulphonyl}piperidin-4- yl)[3-(morpholin-4- ylmethyl)phenyl]methanol |
| 101 | 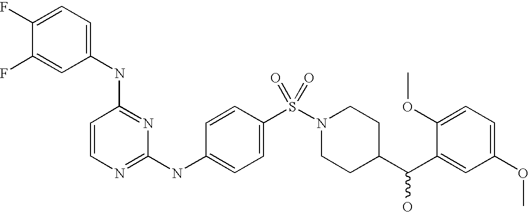 | 612 | (1-{[4-({4-[(3,4- difluorophenyl)- amino]pyrimidin-2- yl}amino)-phenyl]- sulphonyl}piperidin-4- yl)(2,5- dimethoxyphenyl)methanol |

The ketones can be obtained according to the following synthesis scheme:

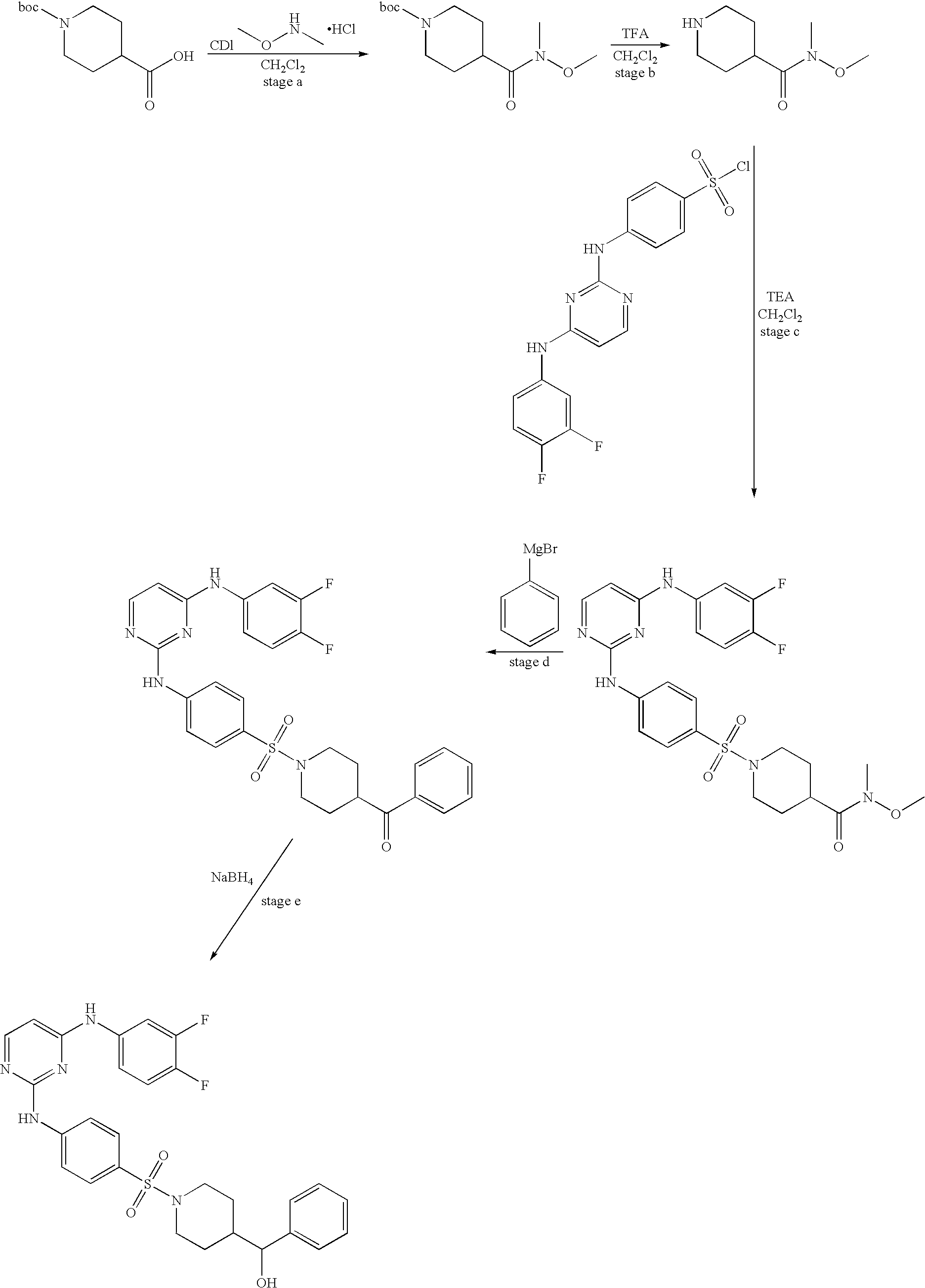

Stage d:

20 mg of the N-methyl-N-methoxyamide obtained in stage c are solubilized in 5 ml of THF and then four equivalents of commercially available solution of phenylmagnesium bromide are added. After an overnight period at ambient temperature, the medium is hydrolyzed with a solution of ammonium chloride and extracted with ethyl acetate and then evaporated. After purification by HPLC under basic conditions, 10.7 mg of ketone are obtained.

(M+H) (+)=550

Stage c:

1.21 g of sulphonyl chloride of procedure 1b are added, in several stages, to 2.06 g of amide compound of stage b in 46 ml of methylene chloride and 3.3 ml of triethylamine. After 2 h at ambient temperature, the reaction medium is evaporated to dryness and the white solid obtained is rinsed twice with 30 ml of methylene chloride to give, after drying, 2.08 g of expected white solid.

NMR: 1.53 (m, 2H); 1.73 (m, 2H); 2.31 (m, 2H); 2.62 (m, 1H); 3.04 (s, 3H); 3.59 (s, 3H); 3.61 (m, 2H); 6.32 (d, J=6.0 Hz, 1H); 7.29 (m, 1H); 7.39 (m, 1H); 7.61 (d, J=8.5 Hz, 2H); 7.99 (d, J=8.5 Hz, 2H); 8.10 (m, 1H); 8.13 (d, J=6.0 Hz, 1H); 9.68 (s, 1H); 9.79 (s, 1H).

(M+H) (+)=533

This intermediate compound constitutes by itself one of the examples of the present invention (Example 104).

Stage b:

25 ml of trifluoroacetic acid are added, under an inert atmosphere, to 2.6 g of the amide of stage a in 25 ml of methylene chloride. After 3 h at ambient temperature, the medium is evaporated to dryness and then loaded, in solution in methanol, onto a Varian Mega Bond Elut SCX cartridge. After elution with pure methanol, the expected product is subsequently released by elution with a 7N solution of ammonia in methanol. After evaporation to dryness, 1.64 g of yellow oil are thus obtained.

Stage e:

1.78 g of carbonyldiimidazole are added, in several stages, to 2.29 g of commercially available N—BOC isonipecotic acid in 40 ml of methylene chloride, and the whole mixture is stirred for 2.5 h at ambient temperature. 1.072 g of N,O-dimethoxyhydroxylamine hydrochloride are then added and the reaction is stirred at ambient temperature overnight. The medium is washed with water, then 0.01N HCl, then NaHCO₃, then again with water. After drying and evaporation, the crude product is purified on a silica cartridge, elution being carried out with a 9/1 and then 8/2 mixture of methylene chloride/ethyl acetate.

2.67 g of the expected product are obtained.

For example, the following compounds are prepared:

| Ex | STRUCTURE | MH+ | NAME |
|---|---|---|---|
| 102 | 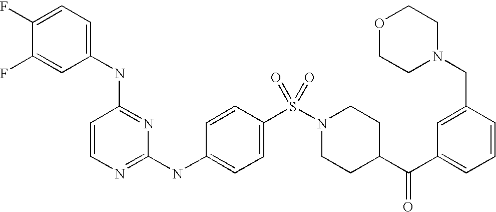 | 649 | (1-{[4-({4-[(3,4- difluorophenyl)amino]pyrimidin-2- yl}amino)phenyl]sulphonyl}piperidin-4- yl)[3-(morpholin-4- ylmethyl)phenyl]methanone |
| 103 | 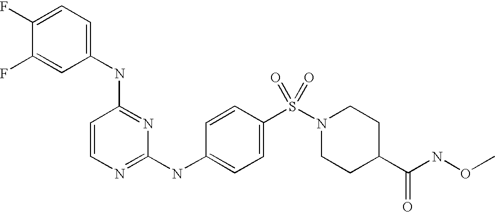 | 519 | 1-{[4-({4-[(3,4- difluorophenyl)amino]pyrimidin-2- yl}amino)phenyl]sulphonyl}-N- methoxypiperidine-4-carboxamide |
| 104 | 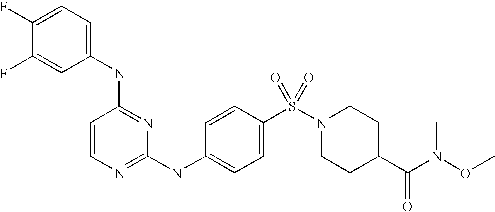 | 533 | 1-{[4-({4-[(3,4- difluorophenyl)amino]pyrimidin-2- yl}amino)phenyl]sulphonyl}-N- methoxy-N-methylpiperidine-4- carboxamide |

### Example 105

### (1-{[4-({4-[(4-Fluorophenyl)amino]pyrimidin-2-yl}amino)phenyl]sulphonyl}piperidin-4-yl)(pyridin-3-yl)-methanamine

Stage 1: tert-butyl 4-[(hydroxyimino) (pyridin-3-yl)methyl]-piperidine-1-carboxylate

290.1 mg of the ketone is solubilized in 20 ml of ethanol. 208.3 mg of commercially available hydroxylamine hydrochloride are added, along with 409.7 mg of NaOAc. The fine suspension obtained is stirred at AT overnight.

The reaction mixture is evaporated in a rotary evaporator under reduced pressure, and the residue is then taken up with H₂O: 30 ml, and extracted with 3×20 ml of EtOAc. The EtOAc phases are combined and the mixture is evaporated in a rotary evaporator. Purification is then carried out by flash chromatography, eluting the product on a 90 g Merck silica cartridge (15-40 μM) with a gradient of CH₂Cl₂/CH₃OH (98-2) in 28 min and then (97-3) in 60 min, with a flow rate of 20 ml/min and detection at 254 nm. The homogeneous fractions collected are evaporated together in a rotary evaporator under reduced pressure. 116 mg of white powder corresponding to the expected Z isomer, and also 169 mg of a second compound corresponding to the E isomer, are obtained.

Stage 2: tert-butyl 4-[amino(pyridin-3-yl)methyl]piperidine-1-carboxylate

2 ml of glacial acetic acid and 2 ml of water are added to a solution of 160 mg of the oxime derivative obtained in stage 1 (E isomer) in 2 ml of EtOH. 171.3 mg of powdered zinc are added to the solution obtained. The suspension is agitated by ultrasound overnight.

The reaction mixture is evaporated in a rotary evaporator under reduced pressure, the residue is then taken up with methanol, and the methanolic solution is loaded onto a 10 g Varian Bond Elut SCX cartridge preconditioned with MeOH. After binding of the product, elution with a solution of CH₃OH/NH₃ (2N), and then evaporation in a rotary evaporator under reduced pressure, 123 mg of a white powder corresponding to the expected product are obtained.

Stage 3: 1-Piperidin-4-yl-1-pyridin-3-ylmethanamine

234 mg of the compound obtained in stage 2 are solubilized in 5 ml of DCM and 3 ml of CF₃CO₂H are added. The clear yellow solution obtained is stirred at AT for 2 hours and is then evaporated in a rotary evaporator under reduced pressure. The residue is taken up with MeOH and the methanolic solution is loaded onto a 5 g Varian Bond elut SCX cartridge preconditioned with MeOH. After binding of the product, elution is carried out with a solution of CH₃OH/NH₃ (2N), and the resulting product is then evaporated in a rotary evaporator under reduced pressure. 133 mg (87%) of a yellow oil, corresponding to the expected product, are obtained.

Stage 4: (1-{[4-({4-[(4-Fluorophenyl)amino]pyrimidin-2-yl)-amino)phenyl]sulphonyl}piperidin-4-yl)(pyridin-3-yl)methanamine

273 mg of the sulphonyl chloride of procedure 1c and 133 mg of amine obtained in the stage 3 are suspended in 10 ml of CH₂Cl₂. 447.0 μl of triethylamine are run in, the mixture is left to stir at AT for one week, and then the solvent is evaporated off in a rotary evaporator under reduced pressure. The residual product is chromatographed on a 25 g Merck silica cartridge (15-40 μM), elution being carried out with a CH₂Cl₂/CH₃OH (90-10) gradient, with a flow rate of 30 ml/min and detection at 254 nm. 113 mg of a white powder corresponding to the expected product are obtained.

### Examples 106 and 107

According to the protocol described in stage 4 of Example 105, the products of Examples 106 and 107 are obtained using the compound of procedure 1a and, respectively, the commercially available amines (R)-phenyl-1-piperidine-4-methanamine and (S)-phenyl-1-piperidine-4-methanamine.

| Ex | STRUCTURE | MH+ | NAME |
|---|---|---|---|
| 105 | 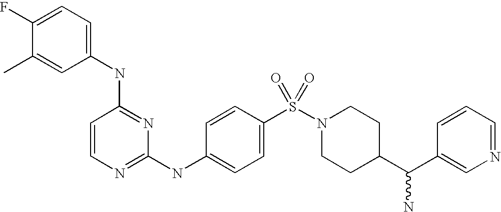 | 533 | (1-{[4-({4-[(4- fluorophenyl)amino]pyrimidin-2- yl}amino)phenyl]sulphonyl}piperidin-4- yl)(pyridin-3-yl)-methanamine |
| 106 | 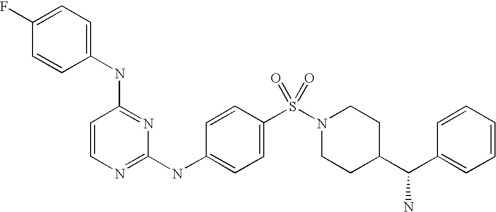 | 533 | (1-{[4-({4-[(4- fluorophenyl)amino]pyrimidin-2- yl}amino)phenyl]sulphonyl}piperidin-4- yl)(phenyl)-R-methanamine |
| 107 | 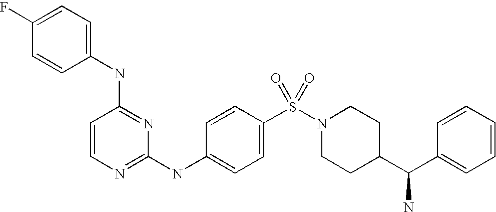 | 548 | (1-{[4-({4-[(4- fluorophenyl)amino]pyrimidin-2- yl}amino)phenyl]sulphonyl}piperidin-4- yl)(phenyl)-S-methanamine |

### Examples 108 to 127

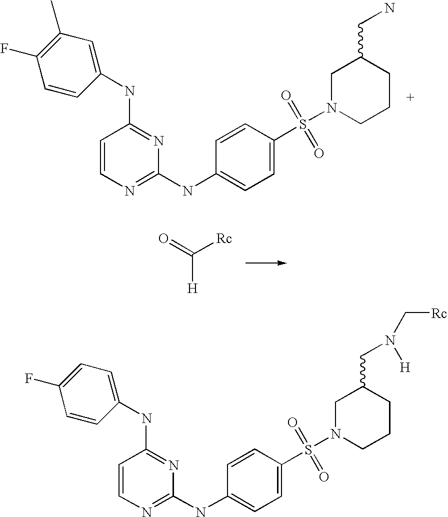

According to the procedure for preparing Examples 50-78, reaction of the sulphonamide of procedure 4i with the appropriate commercially available aldehydes gives the following products (20 examples in the table below which constitute Examples 108 to 127 of the present invention). The expected products are described in the form of a trifluoroacetic acid salt.

| Ex | Structure | nomenclature | [M + H] | |
|---|---|---|---|---|
| 108 | 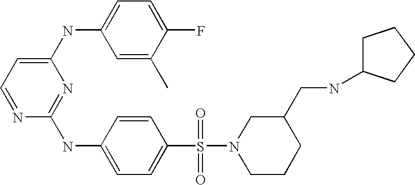 | N*2*-[4-(3-cyclopentylaminomethyl- piperidine-1-sulphonyl)phenyl]-N*4*- (4-fluoro-3-methylphenyl)pyrimidine- 2,4-diamine | 539.28 | R, S |
| 109 | 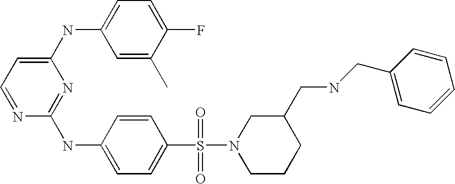 | N*2*-{4-[3-(benzylamino- methyl)piperidine-1-sulphonyl]phenyl}- N*4*-((Z)-4-fluoro-3-methyl-1- methylenepenta-2,4-dienyl)pyrimidine- 2,4-diamine | 561.27 | R, S |
| 110 | 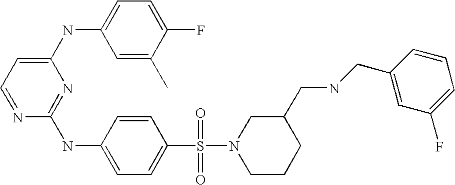 | N*2*-(4-{3-[(3-fluorobenzylamino)- methyl]piperidine-1-sulphonyl}- phenyl)-N*4*-(4-fluoro-3- methylphenyl)pyrimidine-2,4-diamine | 579.27 | R, S |
| 111 | 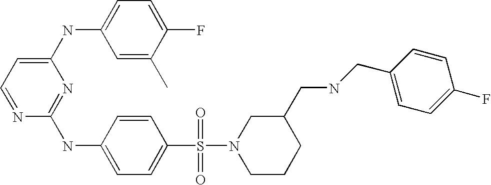 | N*2*-(4-{3-[(4-fluorobenzylamino)- methyl]piperidine-1-sulphonyl}- phenyl)-N*4*-(4-fluoro-3- methylphenyl)pyrimidine-2,4-diamine | 579.27 | R, S |
| 112 | 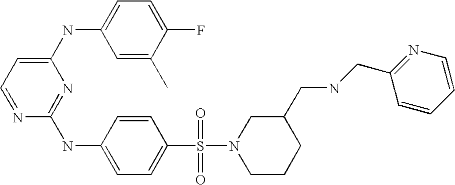 | N*4*-(4-fluoro-3-methylphenyl)-N*2*- [4-(3-{[(pyridin-2- ylmethyl)amino]methyl}piperidine-1- sulphonyl)phenyl]pyrimidine-2,4- diamine | 562.28 | R, S |
| 113 | 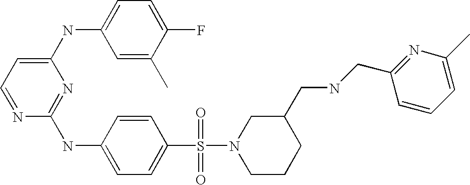 | N*4*-(4-fluoro-3-methylphenyl)-N*2*- [4-(3-{[(6-methylpyridin-2- ylmethyl)amino]methyl}piperidine-1- sulphonyl)phenyl]pyrimidine-2,4- diamine | 576.31 | R, S |
| 114 | 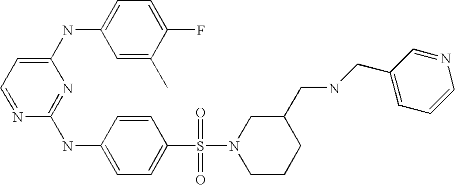 | N*4*-(4-fluoro-3-methylphenyl)-N*2*- [4-(3-{[(pyridin-3- ylmethyl)amino]methyl}piperidine-1- sulphonyl)phenyl]pyrimidine-2,4- diamine | 562.34 | R, S |
| 115 | 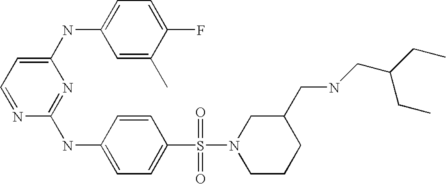 | N*2*-(4-{3-[(2-ethylbutylamino)- methyl]piperidine-1-sulphonyl}- phenyl)-N*4*-(4-fluoro-3- methylphenyl)pyrimidine-2,4-diamine | 555.32 | R, S |
| 116 | 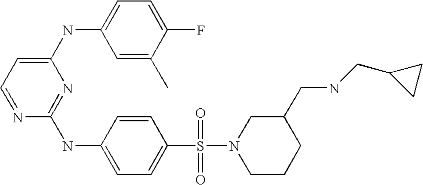 | N*2*-(4-{3- [(cyclopropylmethylamino)- methyl]piperidine-1-sulphonyl}- phenyl)-N*4*-(4-fluoro-3- methylphenyl)pyrimidine-2,4-diamine | 525.28 | R, S |
| 117 | 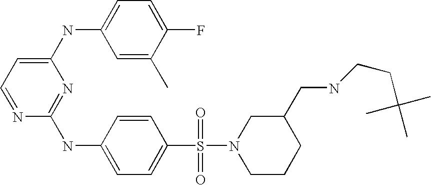 | N*2*-(4-{3-[(3,3-dimethylbutylamino)- methyl]piperidine-1-sulphonyl}- phenyl)-N*4*-(4-fluoro-3- methylphenyl)pyrimidine-2,4-diamine | 555.33 | R, S |
| 118 | 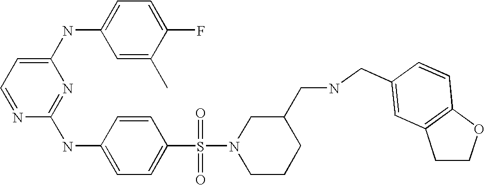 | N*2*-[4-(3-{[(2,3-dihydrobenzofuran- 5-ylmethyl)amino]methyl}piperidine-1- sulphonyl)phenyl]-N*4*-(4-fluoro-3- methylphenyl)pyrimidine-2,4-diamine | 603.31 | R, S |
| 119 | 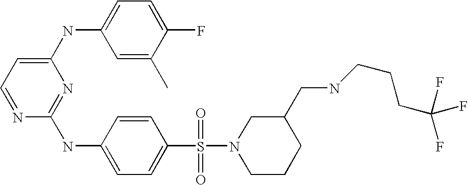 | N*4*-(4-fluoro-3-methylphenyl)-N*2*- (4-{3-[(4,4,4-trifluorobutylamino)- methyl]piperidine-1-sulphonyl}- phenyl)pyrimidine-2,4-diamine | | |
| 120 | 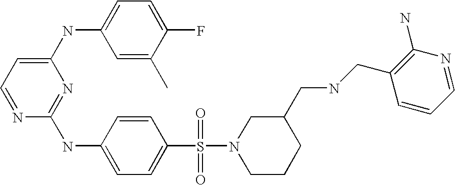 | N*2*-[4-(3-{[(2-aminopyridin-3- ylmethyl)amino]methyl}piperidine-1- sulphonyl)phenyl]-N*4*-(4-fluoro-3- methylphenyl)pyrimidine-2,4-diamine | 577.32 | R, S |
| 121 | 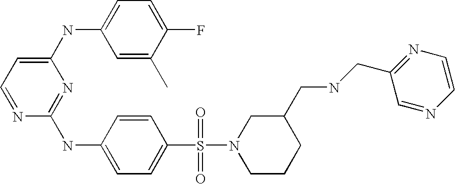 | N*4*-(4-fluoro-3-methylphenyl)-N*2*- [4-(3-{[(pyrazin-2- ylmethyl)amino]methyl}piperidine-1- sulphonyl)-phenyl]pyrimidine-2,4- diamine | 563.28 | R, S |
| 122 | 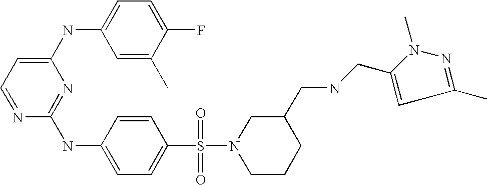 | N*2*-[4-(3-{[(2,5-dimethyl-2H- pyrazol-3- ylmethyl)amino]methyl}piperidine-1- sulphonyl)phenyl]-N*4*-(4-fluoro-3- methylphenyl)pyrimidine-2,4-diamine | 579.32 | R, S |
| 123 | 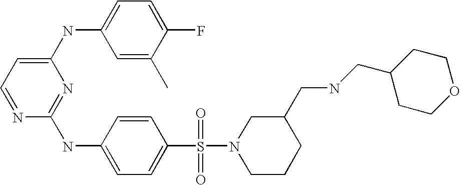 | N*4*-(4-fluoro-3-methylphenyl)-N*2*- [4-(3-{[(tetrahydropyran-4- ylmethyl)amino]methyl}piperidine-1- sulphonyl)phenyl]pyrimidine-2,4- diamine | 569.32 | R, S |
| 124 | 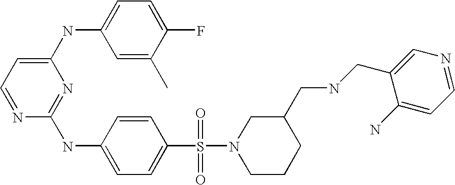 | N*2*-[4-(3-{[(4-aminopyridin-3- ylmethyl)amino]methyl}piperidine-1- sulphonyl)phenyl]-N*4*-(4-fluoro-3- methylphenyl)pyrimidine-2,4-diamine | 577.31 | R, S |
| 125 | 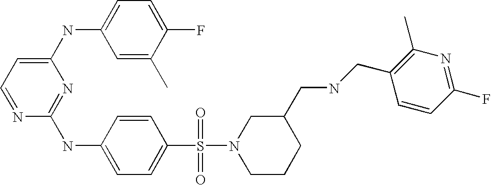 | N*4*-(4-fluoro-3-methylphenyl)-N*2*- [4-(3-{[(6-fluoro-2-methylpyridin-3- ylmethyl)amino]methyl}piperidine-1- sulphonyl)phenyl]pyrimidine-2,4- diamine | 594.30 | R, S |
| 126 | 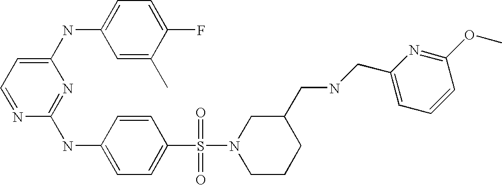 | N*4*-(4-fluoro-3-methylphenyl)-N*2*- [4-(3-{[(6-methoxypyridin-2- ylmethyl)amino]methyl}piperidine-1- sulphonyl)phenyl]pyrimidine-2,4- diamine | 592.30 | R, S |
| 127 | 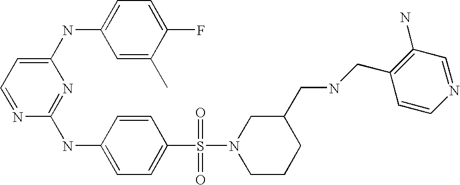 | N*2*-[4-(3-{[(3-aminopyridin-4- ylmethyl)amino]methyl}piperidine-1- sulphonyl)phenyl]-N*4*-(4-fluoro-3- methylphenyl)pyrimidine-2,4-diamine | 577.32 | R, S |

### Examples 128 to 180

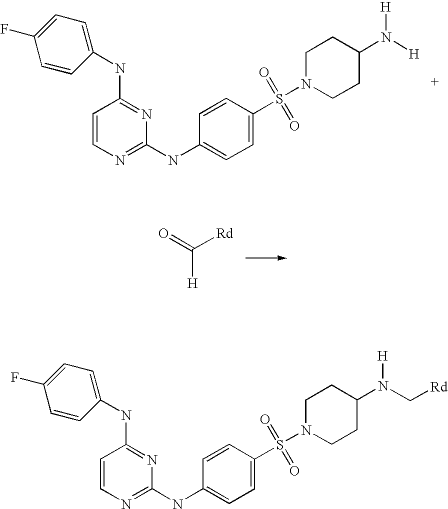

According to the procedure for preparing Examples 128-180, reaction of the sulphonamide of procedure 4h with the appropriate commercially available aldehydes gives the following products (20 examples in the table below which constitute Examples 128 to 180 of the present invention). The expected products are described in the form of a trifluoroacetic acid salt.

| Ex | Structure | Name | MH+ |
|---|---|---|---|
| 128 | 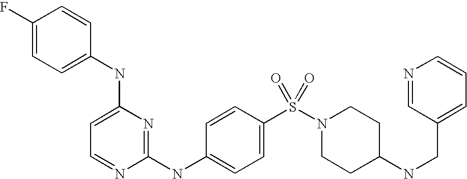 | N4-(4-fluorophenyl)-N2-(4-{4- [(pyridin-3-ylmethyl)amino]piperidine- 1-sulphonyl}-phenyl)pyrimidine-2,4- diamine | 534.15 |
| 129 | 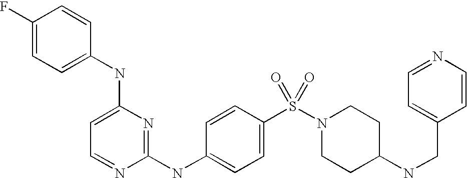 | N4-(4-fluorophenyl)-N2-(4-{4- [(pyridin-4-ylmethyl)amino]piperidine- 1-sulphonyl}-phenyl)pyrimidine-2,4- diamine | 534.15 |
| 130 | 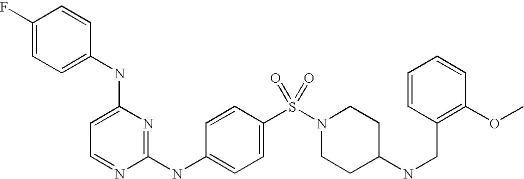 | N4-(4-fluorophenyl)-N2-{4-[4-(2- methoxy-benzylamino)piperidine-1- sulphonyl]phenyl}-pyrimidine-2,4- diamine | 563.17 |
| 131 | 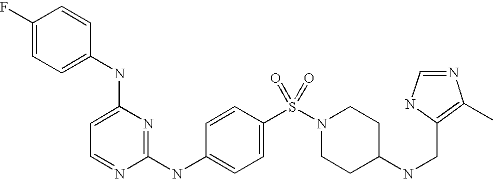 | N4-(4-fluorophenyl)-N2-(4-{4-[(5- methyl-3H-imidazol-4- ylmethyl)amino]piperidine-1- sulphonyl}-phenyl)pyrimidine-2,4- diamine | 537.17 |
| 132 | 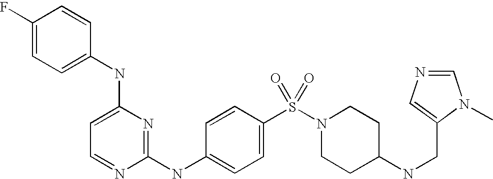 | N4-(4-fluorophenyl)-N2-(4-{4-[(3- methyl-3H-imidazol-4- ylmethyl)amino]piperidine-1- sulphonyl}-phenyl)pyrimidine-2,4- diamine | 537.17 |
| 133 | 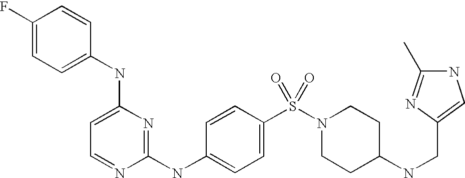 | N4-(4-fluorophenyl)-N2-(4-{4-[(2- methyl-1H-imidazol-4- ylmethyl)amino]piperidine-1- sulphonyl}phenyl)pyrimidine-2,4- diamine | 537.17 |
| 134 | 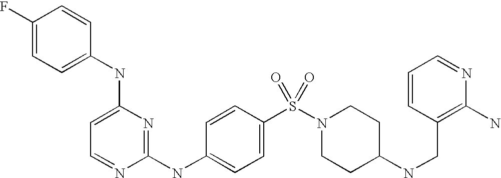 | N2-(4-{4-[(2-aminopyridin-3- ylmethyl)amino]piperidine-1- sulphonyl}-phenyl)-N4-(4- fluorophenyl)pyrimidine-2,4-diamine | 549.17 |
| 135 | 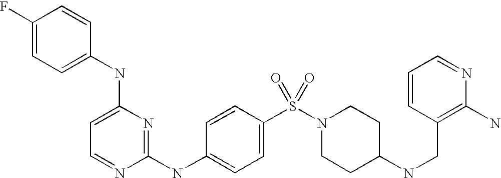 | N2-(4-{4-[(2-aminopyridin-3-ylmethyl)- amino]piperidine-1-sulphonyl}phenyl)- N4-(4-fluorophenyl)pyrimidine-2,4- diamine | 549.17 |
| 136 | 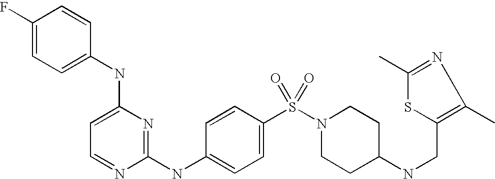 | N2-(4-{4-[(2,4-dimethylthiazol-5- ylmethyl)amino]piperidine-1- sulphonyl}-phenyl)-N4-(4- fluorophenyl)pyrimidine-2,4-diamine | 568.15 |
| 137 | 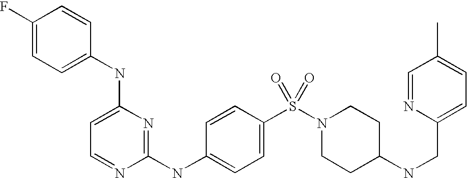 | N4-(4-fluorophenyl)-N2-(4-{4-[(5- methylpyridin-2- ylmethyl)amino]piperidine-1- sulphonyl}phenyl)pyrimidine-2,4- diamine | 548.19 |
| 138 | 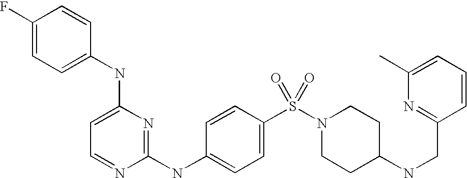 | N4-(4-fluorophenyl)-N2-(4-{4-[(6- methylpyridin-2- ylmethyl)amino]piperidine-1- sulphonyl}phenyl)pyrimidine-2,4- diamine | 548.18 |
| 139 | 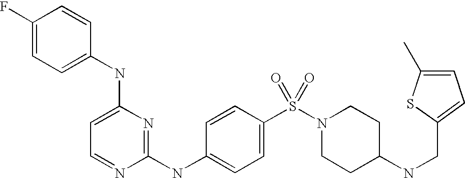 | N4-(4-fluorophenyl)-N2-(4-{4-[(5- methylthiophen-2- ylmethyl)amino]piperidine-1- sulphonyl}-phenyl)pyrimidine-2,4- diamine | 553.14 |
| 140 | 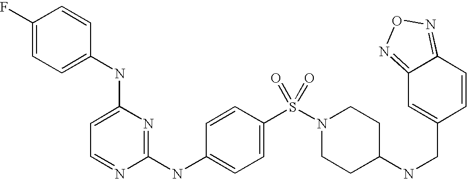 | N2-(4-{4-[(benzo[1,2,5]oxadiazol-5- ylmethyl)amino]piperidine-1- sulphonyl}-phenyl)-N4-(4- fluorophenyl)pyrimidine-2,4-diamine | 575.15 |
| 141 | 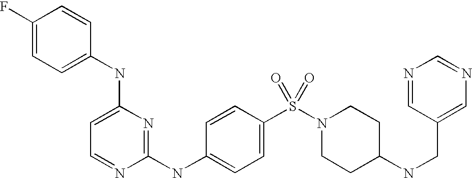 | N4-(4-fluorophenyl)-N2-(4-{4- [(pyrimidin-5- ylmethyl)amino]piperidine-1- sulphonyl}-phenyl)pyrimidine-2,4- diamine | 535.14 |
| 142 | 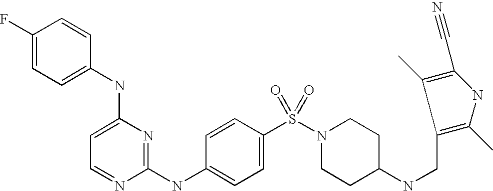 | 4-[(1-{4-[4-(4- fluorophenylamino)pyrimidin-2- ylamino]benzenesulphonyl}piperidin-4- ylamino)methyl]-3,5-dimethyl-1H- pyrrole-2-carbonitrile | 575.19 |
| 143 | 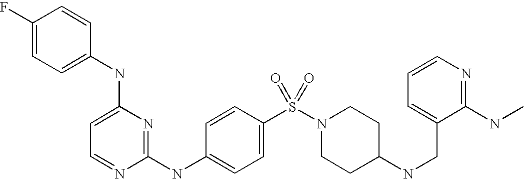 | N4-(4-fluorophenyl)-N2-(4-{4-[(2- methylaminopyridin-3- ylmethyl)amino]piperidine-1- sulphonyl}-phenyl)pyrimidine-2,4- diamine | 563.19 |
| 144 | 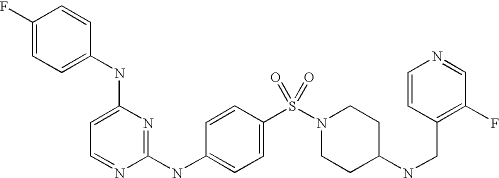 | N4-(4-fluorophenyl)-N2-(4-{4-[(3- fluoro-pyridin-4- ylmethyl)amino]piperidine-1- sulphonyl}phenyl)pyrimidine-2,4- diamine | 552.16 |
| 145 | 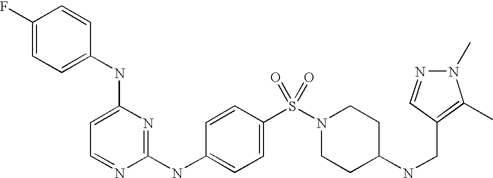 | N2-(4-{4-[(1,5-dimethyl-1H-pyrazol-4- ylmethyl)amino]piperidine-1- sulphonyl}-phenyl)-N4-(4- fluorophenyl)pyrimidine-2,4-diamine | 551.19 |
| 146 | 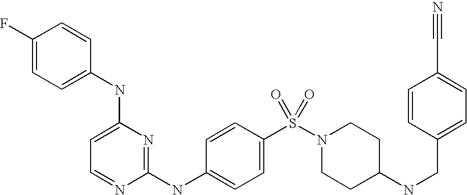 | 4-[(1-{4-[4-(4- fluorophenylamino)pyrimidin-2- ylamino]benzenesulphonyl}piperidin-4- ylamino)methyl]benzonitrile | 558.18 |
| 147 | 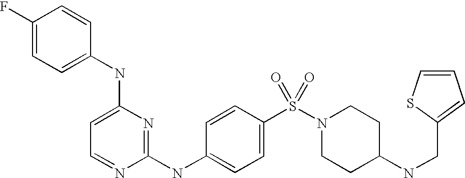 | N4-(4-fluorophenyl)-N2-(4-{4- [(thiophen-2- ylmethyl)amino]piperidine-1- sulphonyl}-phenyl)pyrimidine-2,4- diamine | 539.14 |
| 148 | 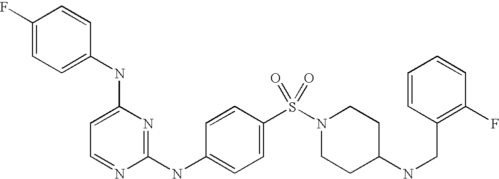 | N2-{4-[4-(2- fluorobenzylamino)piperidine-1- sulphonyl]phenyl}-N4-(4- fluorophenyl)pyrimidine-2,4-diamine | 551.18 |
| 149 | 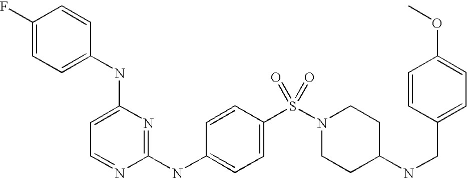 | N4-(4-fluorophenyl)-N2-{4-[4-(4- methoxy-benzylamino)piperidine-1- sulphonyl]phenyl}-pyrimidine-2,4- diamine | 563.16 |
| 150 | 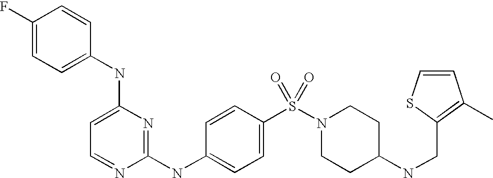 | N4-(4-fluorophenyl)-N2-(4-{4-[(3- methylthiophen-2- ylmethyl)amino]piperidine-1- sulphonyl}-phenyl)pyrimidine-2,4- diamine | 553.16 |
| 151 | 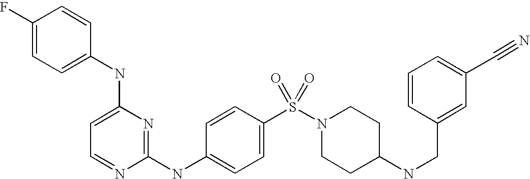 | 3-[(1-{4-[4-(4- fluorophenylamino)benzenesulphonyl} piperidin-4-ylamino)methyl]benzonitrile | 558.18 |
| 152 | 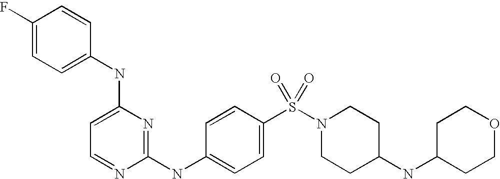 | N4-(4-fluorophenyl)-N2-{4-[4- (tetrahydro-pyran-4-ylamino)piperidine- 1-sulphonyl]phenyl}pyrimidine-2,4- diamine | 527.22 |
| 153 | 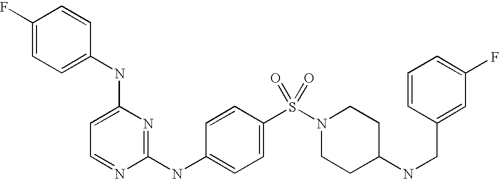 | N2-{4-[4-(3- fluorobenzylamino)piperidine-1- sulphonyl]phenyl}-N4-(4- fluorophenyl)pyrimidine-2,4-diamine | 551.17 |
| 154 | 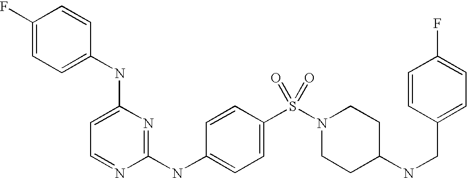 | N2-{4-[4-(4- fluorobenzylamino)piperidine-1- sulphonyl]phenyl}-N4-(4- fluorophenyl)pyrimidine-2,4-diamine | 551.18 |
| 155 | 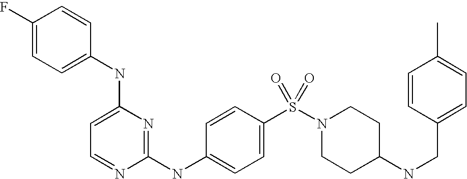 | N4-(4-fluorophenyl)-N2-{4-[4-(4- methyl-benzylamino)piperidine-1- sulphonyl]phenyl}-pyrimidine-2,4- diamine | 547.19 |
| 156 | 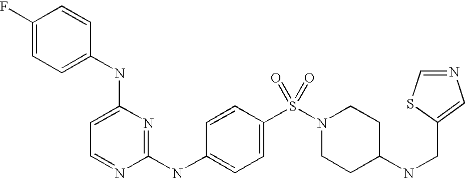 | N4-(4-fluorophenyl)-N2-(4-{4-[(thiazol- 5-ylmethyl)amino]piperidine-1- sulphonyl}-phenyl)pyrimidine-2,4- diamine | 540.15 |
| 157 | 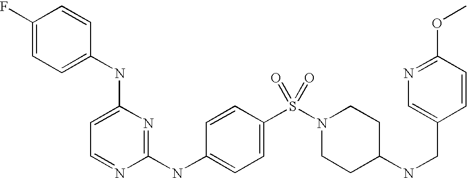 | N4-(4-fluorophenyl)-N2-(4-{4-[(6- methoxy-pyridin-3- ylmethyl)amino]piperidine-1- sulphonyl}phenyl)pyrimidine-2,4- diamine | 564.19 |
| 158 | 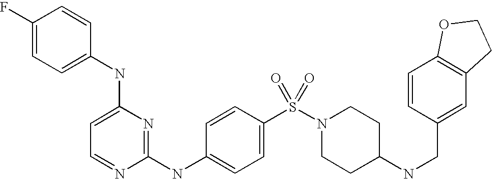 | N2-(4-{4-[(2,3-dihydrobenzofuran-5- ylmethyl)amino]piperidine-1- sulphonyl}-phenyl)-N4-(4- fluorophenyl)pyrimidine-2,4-diamine | 575.21 |
| 159 | 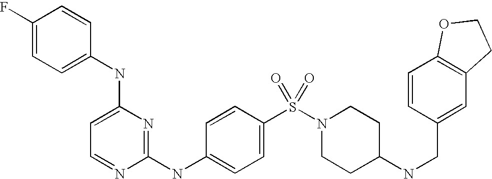 | N2-(4-{4-[(2,3-dihydrobenzofuran-5- ylmethyl)amino]piperidine-1- sulphonyl}-phenyl)-N4-(4- fluorophenyl)pyrimidine-2,4-diamine | 575.21 |
| 160 | 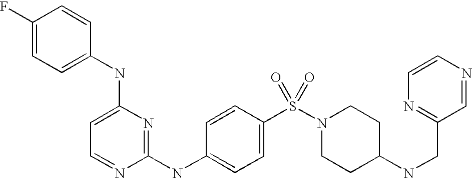 | N4-(4-fluorophenyl)-N2-(4-{4- [(pyrazin-2-ylmethyl)amino]piperidine- 1-sulphonyl}-phenyl)pyrimidine-2,4- diamine | 535.18 |
| 161 | 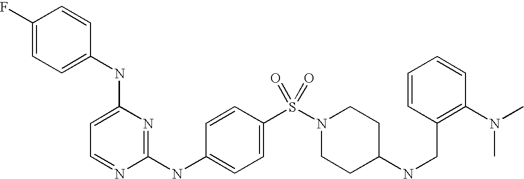 | N2-{4-[4-(2-dimethylamino- benzylamino)piperidine-1- sulphonyl]phenyl}-N4-(4- fluorophenyl)pyrimidine-2,4-diamine | 576.22 |
| 162 | 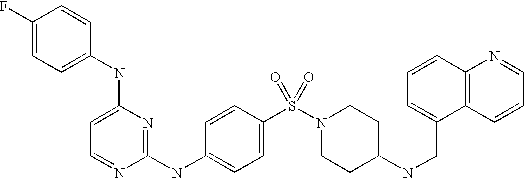 | N4-(4-fluorophenyl)-N2-(4-{4- [(quinolin-5-ylmethyl)amino]piperidine- 1-sulphonyl)-phenyl)pyrimidine-2,4- diamine | 584.21 |
| 163 | 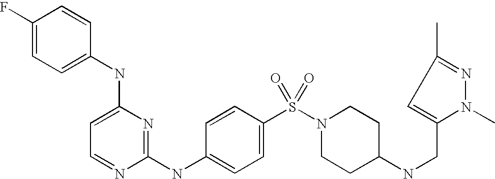 | N2-(4-{4-[(2,5-dimethyl-2H-pyrazol-3- ylmethyl)amino]piperidine-1- sulphonyl}-phenyl)-N4-(4- fluorophenyl)pyrimidine-2,4-diamine | 551.22 |
| 164 | 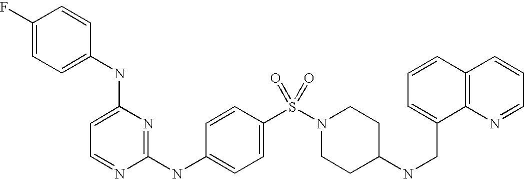 | N4-(4-fluorophenyl)-N2-(4-{4- [(quinolin-8-ylmethyl)amino]piperidine- 1-sulphonyl}-phenyl)pyrimidine-2,4- diamine | 584.2 |
| 165 | 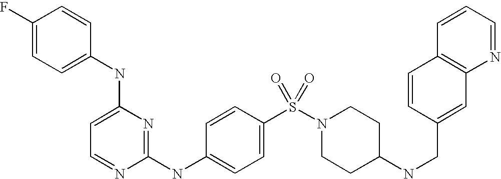 | N4-(4-fluorophenyl)-N2-(4-{4- [(quinolin-7-ylmethyl)amino]piperidine- 1-sulphonyl}-phenyl)pyrimidine-2,4- diamine | 584.21 |
| 166 | 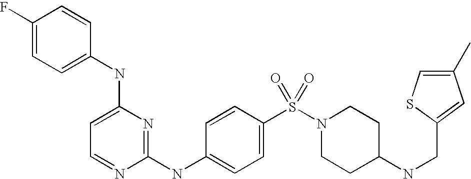 | N4-(4-fluorophenyl)-N2-(4-{4-[(4- methylthiophen-2- ylmethyl)amino]piperidine-1- sulphonyl}-1-phenyl)pyrimidine-2,4- diamine | 553.16 |
| 167 | 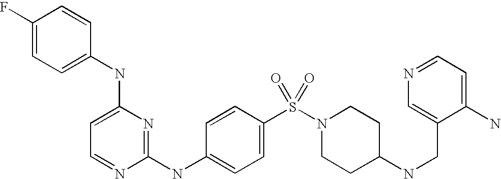 | N2-(4-{4-[(4-aminopyridin-3- ylmethyl)amino]piperidine-1- sulphonyl}-phenyl)-N4-(4- fluorophenyl)pyrimidine-2,4-diamine | 549.21 |
| 168 | 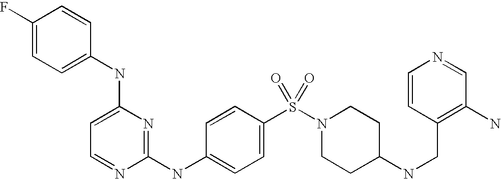 | N2-(4-{4-[(3-aminopyridin-4- ylmethyl)amino]piperidine-1- sulphonyl}-phenyl)-N4-(4- fluorophenyl)pyrimidine-2,4-diamine | 549.21 |
| 169 | 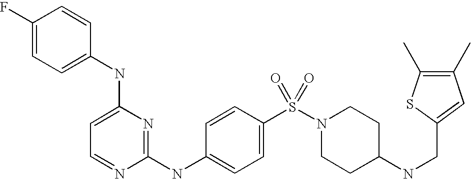 | N2-(4-{4-[(4,5-dimethylthiophen-2- ylmethyl)amino]piperidine-1- sulphonyl}-phenyl)-N4-(4- fluorophenyl)pyrimidine-2,4-diamine | 567.17 |
| 170 | 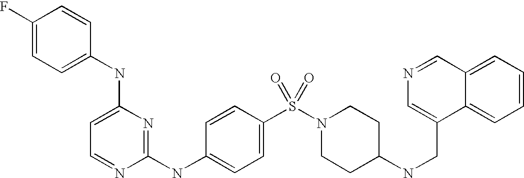 | N4-(4-fluorophenyl)-N2-(4-{4- [(isoquinolin-4- ylmethyl)amino]piperidine-1- sulphonyl}-phenyl)pyrimidine-2,4- diamine | 584.2 |
| 171 | 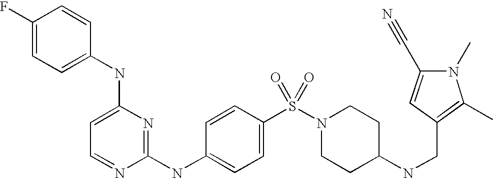 | 4-[(1-{4-[4-(4- fluorophenylamino)pyrimidin-2- ylamino]benzenesulphonyl}piperidin-4- ylamino)methyl]-1,5-dimethyl-1H- pyrrole-2-carbonitrile | 575.22 |
| 172 | 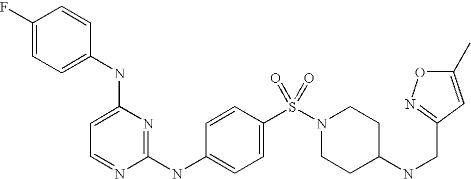 | N4-(4-fluorophenyl)-N2-(4-{4-[(5- methyl-isoxazol-3- ylmethyl)amino]piperidine-1- sulphonyl}phenyl)pyrimidine-2,4- diamine | 538.17 |
| 173 | 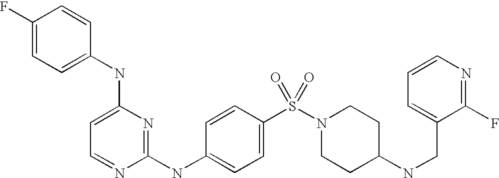 | N4-(4-fluorophenyl)-N2-(4-{4-[(2- fluoro-pyridin-3- ylmethyl)amino]piperidine-1- sulphonyl}phenyl)pyrimidine-2,4- diamine | 552.17 |
| 174 | 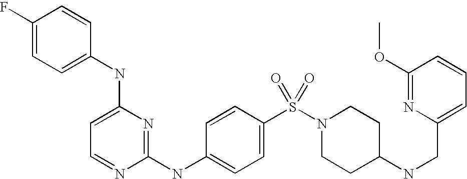 | N4-(4-fluorophenyl)-N2-(4-{4-[(6- methoxy-pyridin-2- ylmethyl)amino]piperidine-1- sulphonyl}phenyl)pyrimidine-2,4- diamine | 564.15 |
| 175 | 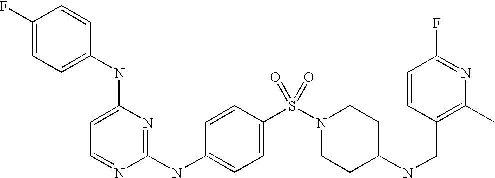 | N2-(4-{4-[(6-fluoro-2-methylpyridin-3- ylmethyl)amino]piperidine-1- sulphonyl}-phenyl)-N4-(4- fluorophenyl)pyrimidine-2,4-diamine | 566.18 |
| 176 | 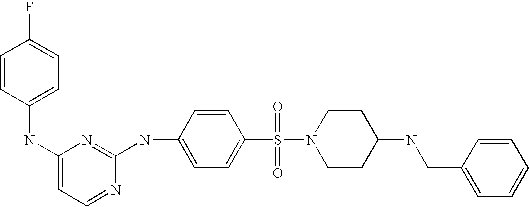 | N2-[4-(4-benzylaminopiperidine-1- sulphonyl)phenyl]-N4-(4- fluorophenyl)pyrimidine-2,4-diamine | 533.18 |
| 177 | 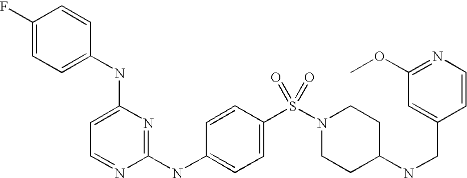 | N4-(4-fluorophenyl)-N2-(4-{4-[(2- methoxy-pyridin-4- ylmethyl)amino]piperidine-1- sulphonyl}phenyl)pyrimidine-2,4- diamine | 564.18 |
| 178 | 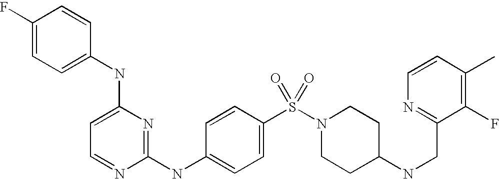 | N2-(4-{4-[(3-fluoro-4-methylpyridin-2- ylmethyl)amino]piperidine-1- sulphonyl}-phenyl)-N4-4- fluorophenyl)pyrimidine-2,4-diamine | 566.18 |
| 179 | 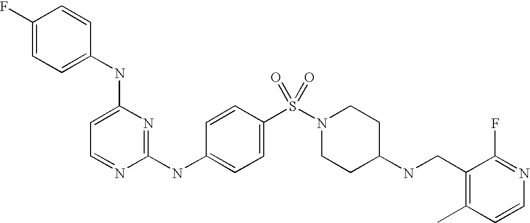 | N2-(4-{4-[(2-fluoro-4-methylpyridin-3- ylmethyl)amino]piperidine-1- sulphonyl}-phenyl)-N4-(4- fluorophenyl)pyrimidine-2,4-diamine | 566.18 |
| 180 | 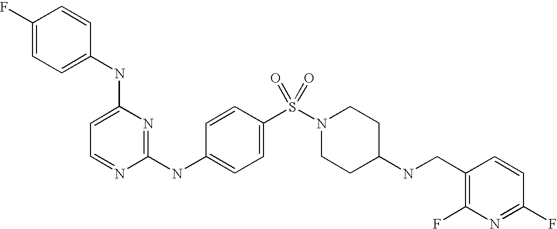 | N2-(4-{4-[(2,6-difluoro-pyridin-3- ylmethyl)amino]piperidine-1- sulphonyl}-phenyl)-N4-(4- fluorophenyl)pyrimidine-2,4-diamine | 570.15 |

### Examples 181 to 260

The products of Examples 181 to 260 are synthesized from the corresponding sulphonyl chloride hydrochlorides 1a-d with the amines described in procedures 5a-f.

| | | | | Mp | |
|---|---|---|---|---|---|
| Ex | Structure | Name | MH+ | (° C.) | |
| 181 | 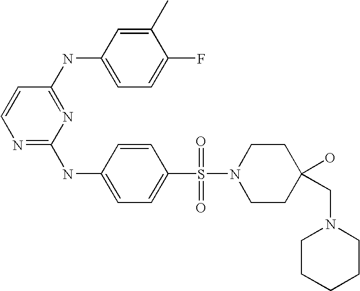 | 1-{4-[4-(4- fluoro-3- methylphenyl- amino)pyrimidin- 2-ylamino] benzenesulphonyl}- 4-piperidin-1- ylmethylpiperidin- 4-ol | 555 | 180 | |
| 182 | 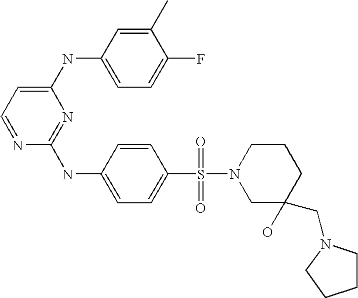 | 1-{4-[4-(4- fluoro-3- methylphenylamino) pyrimidin-2- ylamino]benzene sulphonyl}-3- pyrrolidin-1-ylmethyl- piperidin-3-ol | 541 | 110 | |
| 183 | 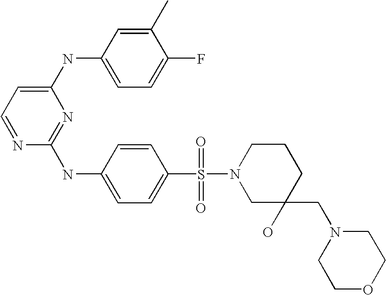 | 1-{4-[4-(4- fluoro-3- methylphenylamino) pyrimidin-2- ylamino]benzene sulphonyl}-3- morpholin-4-ylmethyl- piperidin-3-ol | 557 | 109 | |
| 184 | 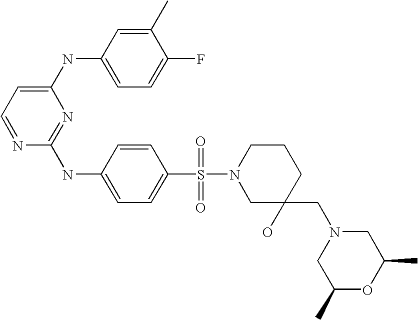 | 3-((2R,6S)-2,6- dimethylmorpholin- 4-ylmethyl)-1- {4-[4-(4-fluoro-3- methylphenylamino) pyrimidin-2- ylamino]benzene sulphonyl}piperidin- 3-ol | 585 | 107 | |
| 185 | 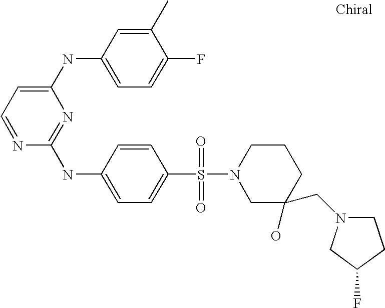 | 1-{4-[4-(4- fluoro-3- methylphenylamino) pyrimidin-2- ylamino]benzene sulphonyl}-3-((S)-3- fluoropyrrolidin-1- ylmethyl)piperidin- 3-ol | 559 | 120 | |
| 186 | 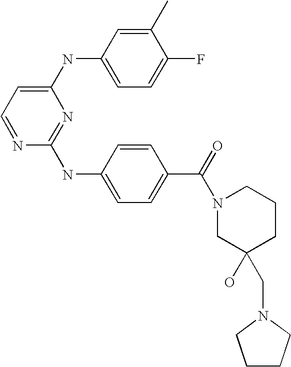 | {4-[4-(4- fluoro-3- methylphenylamino) pyrimidin-2- ylamino]phenyl}- (3-hydroxy-3- pyrrolidin-1- ylmethylpiperidin- 1-yl)methanone | 505.1 | 119 | |
| 187 | 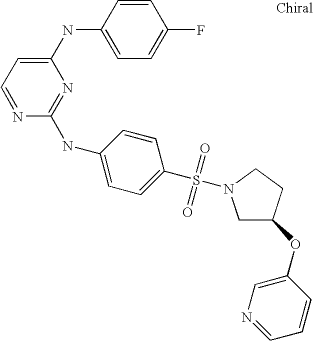 | N4-(4-fluorophenyl)- N2-{4-[(R)-3- (pyridin-3- yloxy)pyrrolidine-1- sulphonyl]phenyl} pyrimidine-2,4- diamine | 507 | 194-196 | [α] D = +18 (MeOH) |
| 188 | 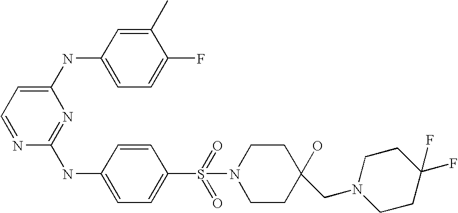 | 4-(4,4-difluoro- piperidin-1- ylmethyl)-1-{4-[4- (4-fluoro-3-methyl- phenylamino) pyrimidin-2- ylamino]benzene sulphonyl}piperidin- 4-ol | 591 | | |
| 189 | 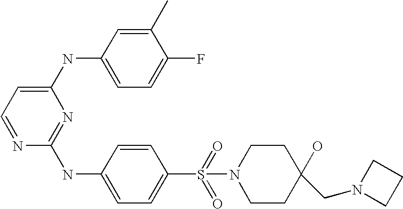 | 4-azetidin-1- ylmethyl-1-{4- [4-(4-fluoro-3- methylphenylamino) pyrimidin-2- ylamino]benzene sulphonyl}piperidin- 4-ol | 527 | | |
| 190 | 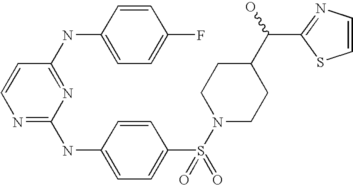 | (1-{4-[4-(4- fluorophenylamino) pyrimidin-2- ylamino]benzene sulphonyl}piperidin- 4-yl)thiazol- 2-ylmethanol | 541 | 195.5 | R, S |
| 191 | 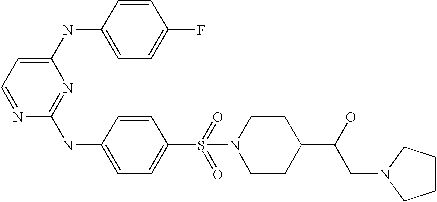 | 1-(1-{4-[4-(4- fluorophenylamino) pyrimidin-2- ylamino]benzene sulphonyl}piperidin- 4-yl)-2- pyrrolidin-1- ylethanol | 541 | | R, S |
| 192 | 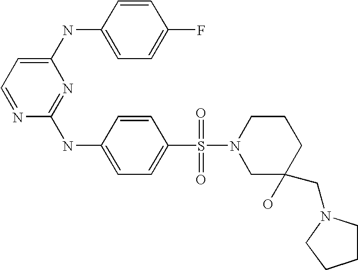 | 1-{4-[4-(4- fluorophenylamino) pyrimidin-2- ylamino]benzene sulphonyl}-3- pyrrolidin-1- ylmethylpiperidin- 3-ol | 527 | 113 | R, S |
| 193 | 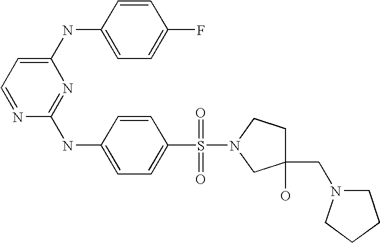 | 1-{4-[4-(4- fluorophenylamino) pyrimidin-2- ylamino]benzene sulphonyl}-3- pyrrolidin-1- ylmethylpyrrolidin- 3-ol | 513 | 112 | R, S |
| 194 | 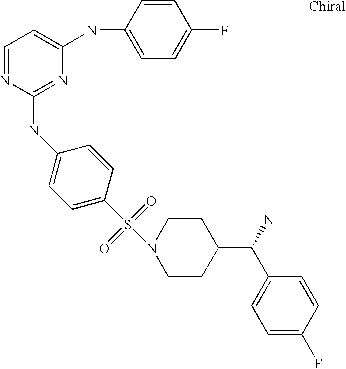 | N2-(4-{4-[(S)- amino(4-fluoro- phenyl)-methyl] piperidin-1- sulphonyl}phenyl)- N4-(4-fluorophenyl) pyrimidine-2,4- diamine | 551 | 146-148 | [α] D = +68 (MeOH) |
| 195 | 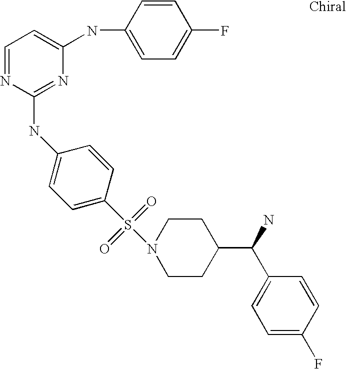 | N2-(4-{4-[(R)- amino-(4-fluoro- phenyl)-methyl] piperidin-1- sulphonyl}-phenyl)- N4-(4-fluorophenyl) pyrimidine-2,4- diamine | 551 | 146-148 | [α] D = −69 (MeOH) |
| 196 | 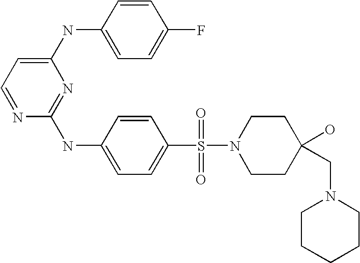 | 1-{4-[4-(4- fluorophenylamino) pyrimidin-2- ylamino]benzene sulphonyl}-4- piperidin-1-ylmethyl- piperidin-4-ol | 541 | 142 | |
| 197 | 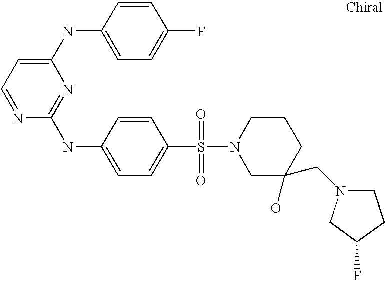 | 1-{4-[4-(4- fluorophenylamino) pyrimidin-2- ylamino]benzene sulphonyl}-3- ((S)-3-fluoro- pyrrolidin-1-ylmethyl) piperidin-3-ol | 545 | 110 | R, S |
| 198 | 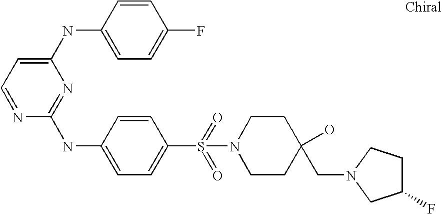 | 1-{4-[4-(4- fluorophenylamino) pyrimidin-2- ylamino]benzene sulphonyl}-4- ((S)-3-fluoro- pyrrolidin-1-ylmethyl) piperidin-4-ol | 545 | 160 | |
| 199 | 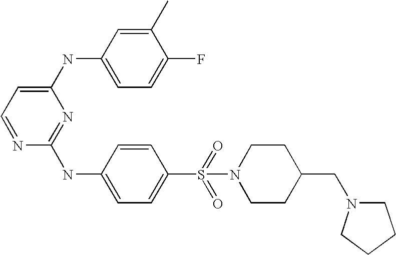 | N4-(4-fluoro-3- methylphenyl)- N2-[4-(4- pyrrolidin-1- ylmethylpiperidin- 1-sulphonyl)phenyl] pyrimidine-2,4- diamine | 525 | 255 | |
| 200 | 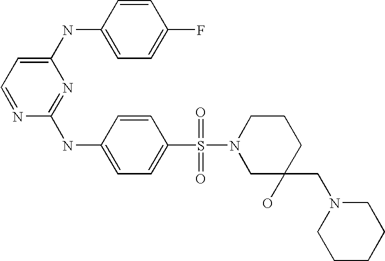 | 1-{4-[4-(4- fluorophenylamino) pyrimidin-2- ylamino]benzene sulphonyl}-3- piperidin-1-ylmethyl- piperidin-3-ol | 541.1 | 109 | R, S |
| 201 | 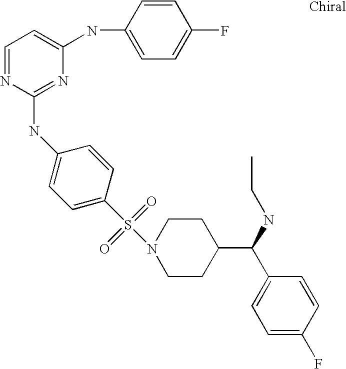 | N2-(4-{4-[(R)- ethylamino-(4- fluorophenyl)methyl] piperidine-1- sulphonyl}phenyl)- N4-(4-fluorophenyl) pyrimidine-2,4- diamine | 579.1 | 136-139 | [α] D = −60 (MeOH) |
| 202 | 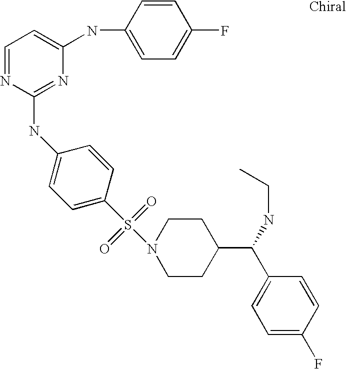 | N2-(4-{4-[(S)- ethylamino-(4- fluorophenyl)methyl] piperidine-1- sulphonyl}phenyl)- N4-(4-fluorophenyl) pyrimidine-2,4- diamine | 579.1 | 137-140 | [α] D = +68 (MeOH) |
| 203 | 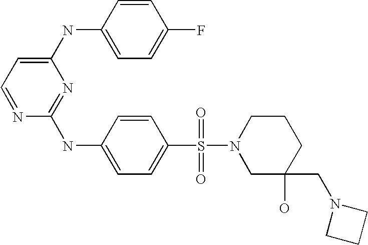 | 3-Azetidin-1- ylmethyl-1-{4-[4-(4- fluorophenylamino) pyrimidin-2- ylamino]benzene sulphonyl}piperidin- 3-ol | 513 | 133 | |
| 204 | 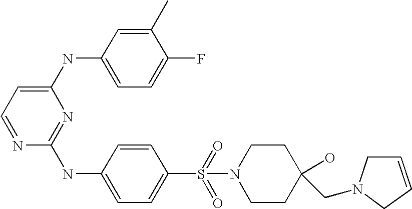 | 4-(2,5-dihydropyrrol- 1-ylmethyl)-1-{4- [4-(4-fluoro-3- methylphenylamino) pyrimidin-2- ylamino]benzene sulphonyl}piperidin- 4-ol | 539.1 | 120 | |
| 205 | 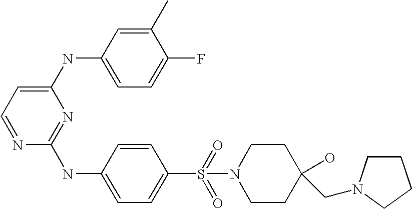 | 1-{4-[4-(4-fluoro-3- methylphenylamino) pyrimidin-2- ylamino]benzene sulphonyl}-4- pyrrolidin-1-ylmethyl- piperidin-4-ol | 541.1 | 169 | |
| 206 | 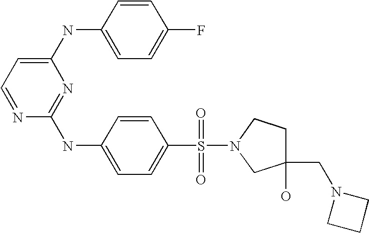 | 3-Azetidin-1- ylmethyl-1-{4-[4-(4- fluorophenylamino) pyrimidin-2- ylamino]benzene sulphonyl}pyrrolidin- 3-ol | 499 | 119 | R, S |
| 207 | 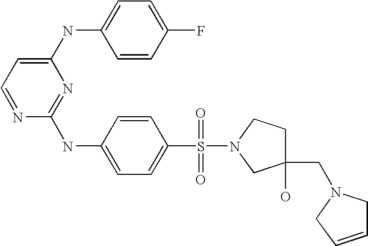 | 3-(2,5-dihydropyrrol- 1-ylmethyl)-1-{4-[4- (4-fluorophenyl- amino)pyrimidin- 2-ylamino]benzene sulphonyl}pyrrolidin- 3-ol | 511 | 103 | R, S |
| 208 | 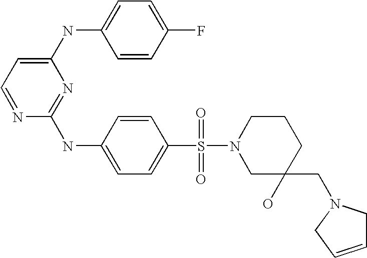 | 3-(2,5-dihydropyrrol- 1-ylmethyl)-1-{4-[4- (4-fluorophenyl- amino)pyrimidin- 2-ylamino]benzene sulphonyl}piperidin- 3-ol | 525 | 110 | R, S |
| 209 | 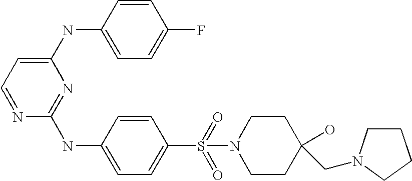 | 1-{4-[4-(4- fluorophenylamino) pyrimidin-2- ylamino]benzene sulphonyl}-4- pyrrolidin-1-ylmethyl- piperidin-4-ol | 527.1 | | |
| 210 | 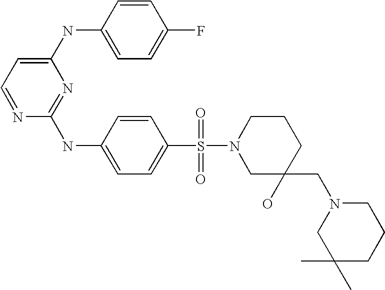 | 3-(3,3-dimethyl- piperidin-1-ylmethyl)- 1-{4-[4-(4- fluorophenylamino) pyrimidin-2- ylamino]benzene sulphonyl}piperidin- 3-ol | 569.2 | 105 | R, S |
| 211 | 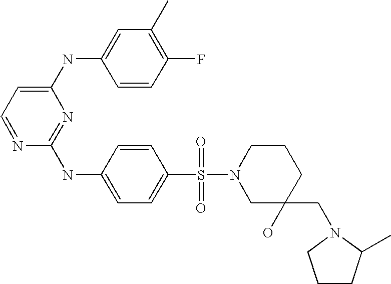 | 1-{4-[4-(4-fluoro-3- methylphenylamino) pyrimidin-2- ylamino]benzene sulphonyl}-3-(2- methylpyrrolidin- 1-ylmethyl)piperidin- 3-ol | 555.2 | 163-166 | R, S |
| 212 | 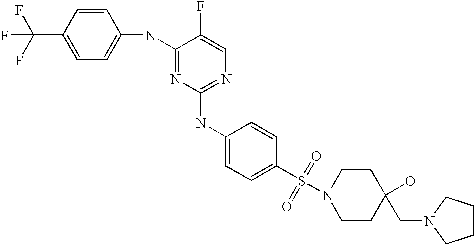 | 1-{4-[5-fluoro-4-(4- trifluoromethylphenyl- amino)pyrimidin-2- ylamino]benzene sulphonyl}-4- pyrrolidin-4-ylmethyl- piperidin-4-ol | 595.1 | 241-243 | |
| 213 | 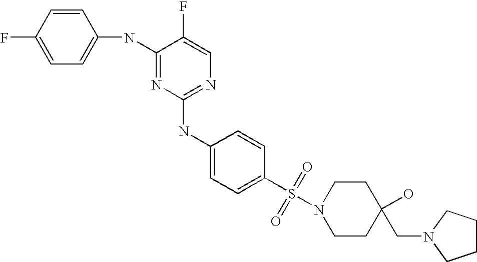 | 1-{4-[5-fluoro-4-(4- fluorophenylamino) pyrimidin-2-ylamino] benzenesulphonyl}-4- pyrrolidin-1-ylmethyl- piperidin-4-ol | 545.2 | 192-194 | |
| 214 | 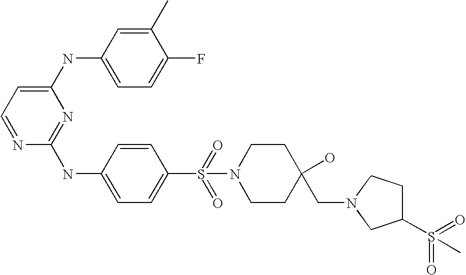 | 1-{4-[4-(4-fluoro-3- methylphenylamino) pyrimidin-2-ylamino] benzenesulphonyl}-4- (3-methanesulphonyl- pyrrolidin-1-ylmethyl) piperidin-4-ol | 619.1 | 138 | |
| 215 | 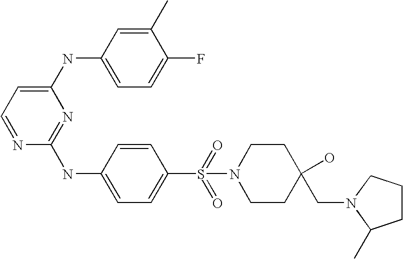 | 1-{4-[4-(4-fluoro-3- methylphenylamino) pyrimidin-2-ylamino] benzenesulphonyl}-4- (2-methylpyrrolidin- 1-ylmethyl)piperidin- 4-ol | 555.1 | 133 | |
| 216 | 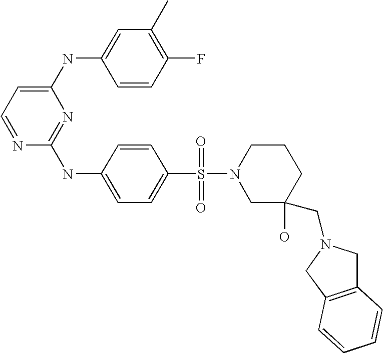 | 3-(1,3-dihydro- isoindol-2-ylmethyl)- 1-{4-[4-(4-fluoro-3- methylphenylamino) pyrimidin-2-ylamino] benzenesulphonyl} piperidin-3-ol | 589.1 | 114 | |
| 217 | 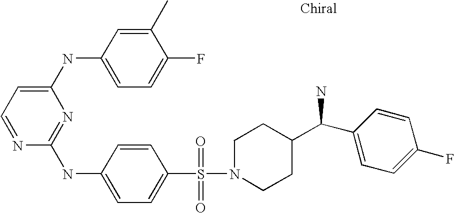 | N2-(4-{4-[(R)-amino- (4-fluorophenyl) methyl]piperidine-1- sulphonyl}phenyl)- N4-(4-fluoro-3- methylphenyl) pyrimidine-2,4- diamine | 565.1 | | [α] D = −50 (MeOH) |
| 218 | 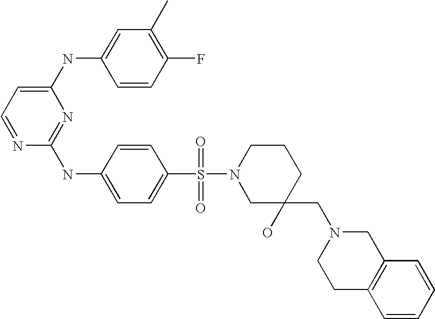 | 3-(3,4-dihydro-1H- isoquinolin-2- ylmethyl)-1-{4-[4-(4- fluoro-3-methyl- phenylamino) pyrimidin-2-ylamino] benzenesulphonyl} piperidin-3-ol | 603.2 | 109 | |
| 219 | 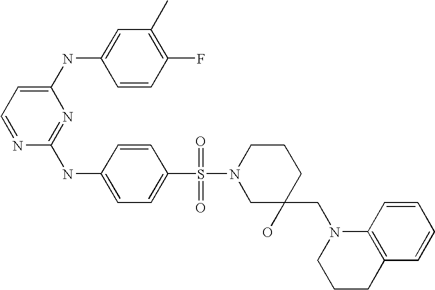 | 3-(3,4-dihydro-2H- quinolin-1-ylmethyl)- 1-(4-[4-(4-fluoro-3- methylphenylamino) pyrimidin-2-ylamino] benzenesulphonyl}- piperidin-3-ol | 603.2 | 115 | |
| 220 | 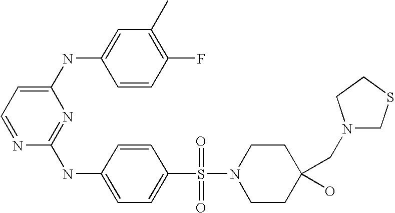 | 1-{4-[4-(4-fluoro-3- methylphenylamino) pyrimidin-2- ylamino]benzene sulphonyl}-4- thiazolidin-3-ylmethyl- piperidin-4-ol | 559.1 | 185 | |
| 221 | 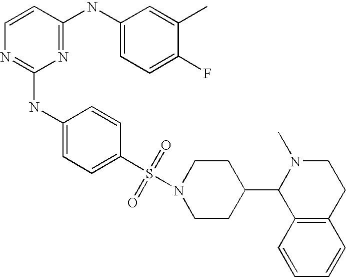 | N4-(4-fluoro-3- methylphenyl)-N2- {4-[4-(2-methyl- 1,2,3,4-tetrahydro- isoquinolin-1- yl)piperidine-1- sulphonyl]phenyl} pyrimidine-2,4- diamine | 587.2 | 147-153 | R, S |
| 222 | 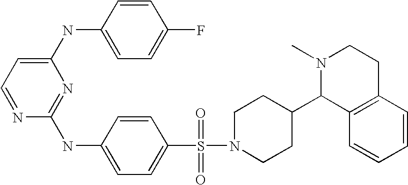 | N4-(4-fluorophenyl)- N2-{4-[4-(2-methyl- 1,2,3,4-tetrahydro- isoquinolin-1- yl)piperidine-1- sulphonyl]phenyl} pyrimidine-2,4- diamine | 573.1 | 166-167 | R, S |
| 223 | 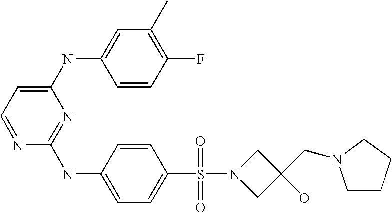 | 1-{4-[4-(4-fluoro-3- methylphenylamino) pyrimidin-2- ylamino]benzene sulphonyl}-3- pyrrolidin-1-ylmethyl- azetidin-3-ol | 513.1 | 137 | |
| 224 | 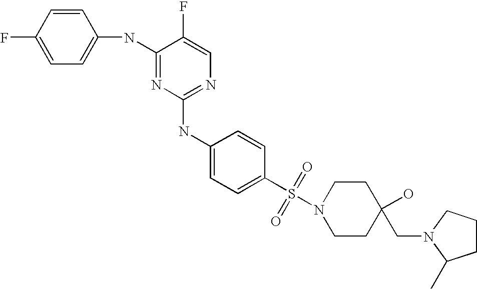 | 1-{4-[5-fluoro-4-(4- fluorophenylamino) pyrimidin-2- ylamino]benzene sulphonyl}-4-(2- methylpyrrolidin-1- ylmethyl)piperidin- 4-ol | 559.1 | 205-210 | R, S |
| 225 | 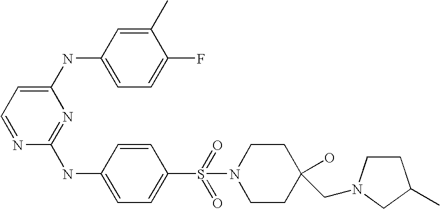 | 1-{4-[4-(4-fluoro-3- methylphenylamino) pyrimidin-2- ylamino]benzene sulphonyl}-4-(3- methylpyrrolidin-1- ylmethyl)piperidin- 4-ol | 555.1 | | |
| 226 | 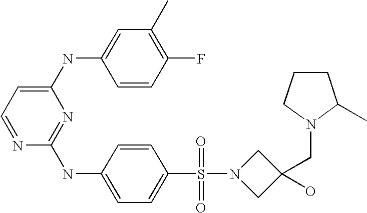 | 1-{4-[4-(4-fluoro-3- methylphenylamino) pyrimidin-2- ylamino]benzene sulphonyl}-3-(2- methylpyrrolidin-1- ylmethyl)-azetidin- 3-ol | 527.1 | 131 | |
| 227 | 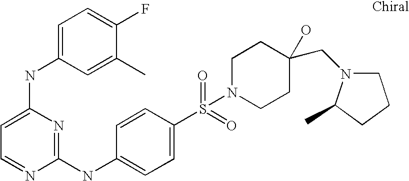 | 1-{4-[4-(4-fluoro-3- methylphenylamino) pyrimidin-2- ylamino]benzene sulphonyl}-4-((R)-2- methylpyrrolidin-1- ylmethyl)piperidin- 4-ol | 552.2 | 114 | [α] D = −36 (MeOH) |
| 228 | 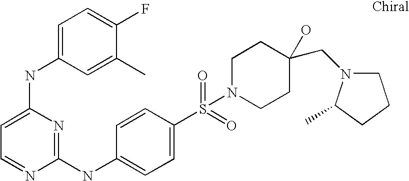 | 1-{4-[4-(4-fluoro-3- methylphenylamino) pyrimidin-2- ylamino]benzene sulphonyl}-4-((S)-2- methylpyrrolidin-1- ylmethyl)piperidin- 4-ol | 555.2 | 114 | [α] D = +16.6 (MeOH) |
| 229 | 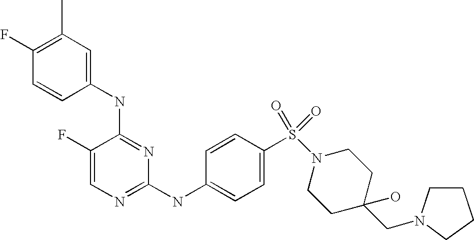 | 1-{4-[5-fluoro-4- (4-fluoro-3-methyl- phenylamino) pyrimidin-2-ylamino] benzenesulphonyl}-4- pyrrolidin-1-ylmethyl- piperidin-4-ol | 559 | 211.5-213.8 | |
| 230 | 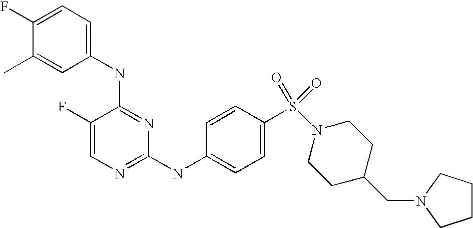 | 5-Fluoro-N4-(4- fluoro-3-methyl- phenyl)-N2-[4-(4- pyrrolidin-1- ylmethyl-piperidine-1- sulphonyl)phenyl] pyrimidine-2,4- diamine | 543 | 221.5-223.7 | |
| 231 | 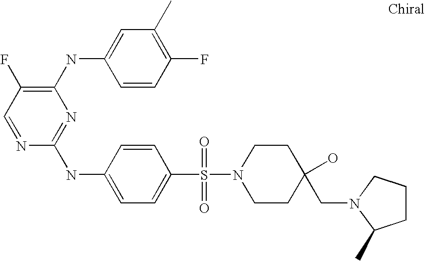 | 1-{4-[5-fluoro-4- (4-fluoro-3-methyl- phenylamino) pyrimidin-2-benzene sulphonyl}-4-((R)-2- methylpyrrolidin-1- ylmethyl)piperidin- 4-ol | 573.1 | 203 | [α] D = −24 (MeOH) |
| 232 | 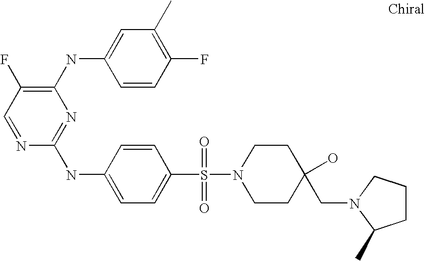 | 1-{4-[5-fluoro-4- (4-fluoro-3-methyl- phenylamino) pyrimidin-2-ylamino] benzenesulphonyl}-4- ((R)-2-methyl- pyrrolidin-1-ylmethyl) piperidin-4-ol | 573.1 | 160 | [α] D = −14 (MeOH) |
| 233 | 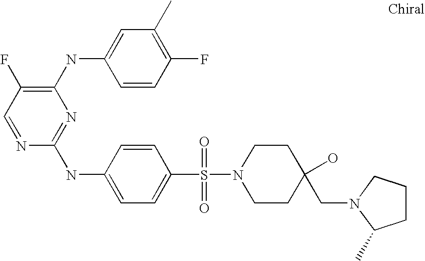 | 1-{4-[5-fluoro-4- (4-fluoro-3-methyl- phenylamino) pyrimidin-2-ylamino] benzenesulphonyl}-4- ((S)-2-methyl- pyrrolidin-1-ylmethyl) piperidin-4-ol | 573.1 | 203 | [α] D = +35 (MeOH) |
| 234 | 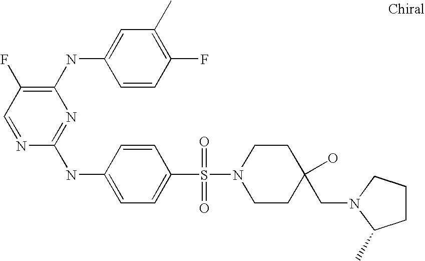 | 1-{4-[5-fluoro-4- (4-fluoro-3-methyl- phenylamino) pyrimidin-2-ylamino] benzenesulphonyl}-4- ((S)-2-methyl- pyrrolidin-1-ylmethyl) piperidin-4-ol | 573.1 | 160 | [α] D = +17 (MeOH) |
| 235 | 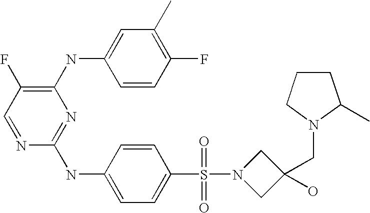 | 1-{4-[5-fluoro-4- (4-fluoro-3-methyl- phenylamino) pyrimidin-2-ylamino] benzenesulphonyl}-3- (2-methylpyrrolidin-1- ylmethyl)azetidin-3-ol | 545 | 176 | |
| 236 | 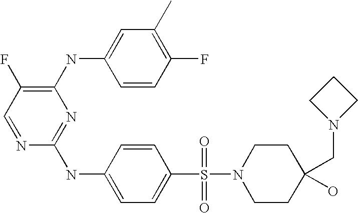 | 4-azetidin-1- ylmethyl-1-{4-[5- fluoro-4-(4-fluoro-3- methylphenylamino) pyrimidin-2-ylamino] benzenesulphonyl} piperidin-4-ol | 545 | 187 | |
| 237 | 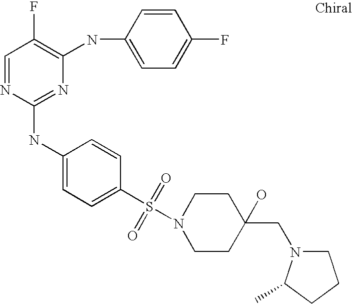 | 1-{4-[5-fluoro-4- (4-fluorophenylamino) pyrimidin-2-ylamino] benzenesulphonyl}-4- ((S)-2-methyl- pyrrolidin-1-ylmethyl) piperidin-4-ol | 559 | 187-197 | [α] D = +17 (MeOH) |
| 238 | 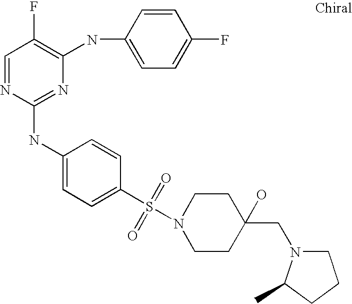 | 1-{4-[5-fluoro-4- (4-fluorophenylamino) pyrimidin-2-ylamino] benzenesulphonyl}-4- ((R)-2-methyl- pyrrolidin-1-ylmethyl) piperidin-4-ol | 559.1 | 186-196 | [α] D = −20 (MeOH) |
| 239 | 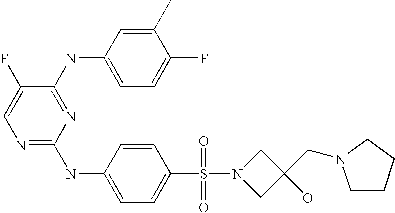 | 1-{4-[5-fluoro-4- (4-fluoro-3-methyl- phenylamino)pyrimidin- 2-ylamino]benzene sulphonyl}-3- pyrrolidin-1-ylmethyl- azetidin-3-ol | 531.1 | | |
| 240 | 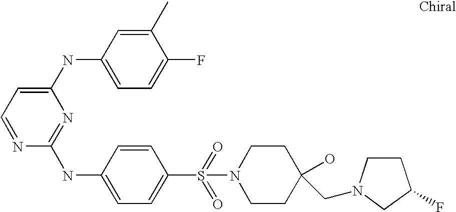 | 1-{4-[4-(4-fluoro-3- methylphenylamino) pyrimidin-1-ylamino] benzenesulphonyl}-4- ((S)-3-fluoro- pyrrolidin-1-ylmethyl) piperidin-4-ol | 509.1 | 509.1 | |
| 241 | 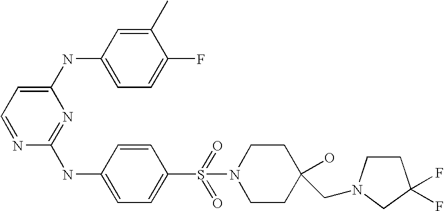 | 4-(3,3-difluoro- pyrrolidin-1-ylmethyl)- 1-{4-[4-(4-fluoro-3- methylphenylamino) pyrimidin-2-ylamino] benzenesulphonyl}- piperidin-4-ol | 577 | 577.1 | |
| 242 | 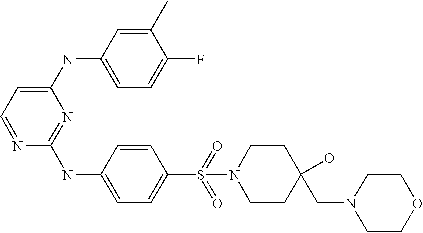 | 1-{4-[4-(4-fluoro-3- methylphenylamino) pyrimidin-2-ylamino] benzenesulphonyl}-4- morpholin-4-ylmethyl- piperidin-4-ol | 557.1 | 557 | |
| 243 | 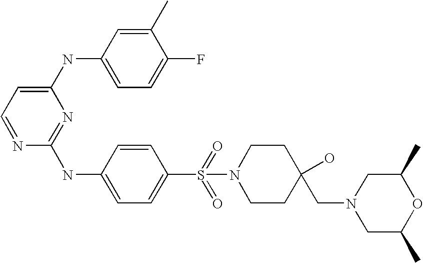 | 4-((2R,6S)-2,6- dimethylmorpholin-4- ylmethyl)-1-{4-[4-(4- fluoro-3-methyl- phenylamino) pyrimidin-2-ylamino] benzenesulphonyl} piperidin-4-ol | 585 | 585.1 | |
| 244 | 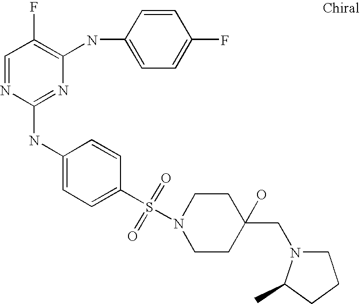 | 1-{4-[5-Fluoro-4- (4-fluorophenylamino) pyrimidin-2-ylamino] benzenesulphonyl}-4- ((R)-2-methyl- pyrrolidin-1-ylmethyl) piperidin-4-ol | 559.1 | 186-196 | [α] D = −20 (MeOH) |
| 245 | 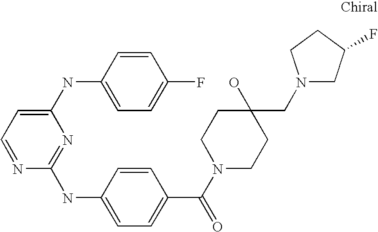 | {4-[4-(4- fluorophenylamino) pyrimidin-2-ylamino] phenyl}-[4-((S)-3- fluoropyrrolidine-1- ylmethyl)-4-hydroxy- piperidin-1-yl] methanone | 509.1 | 212 | |
| 247 | 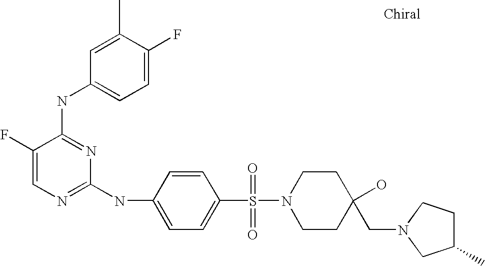 | 1-{4-[5-fluoro-4- (4-fluoro-3-methyl- phenylamino)pyrimidin- 2-ylamino]benzene sulphonyl}-4- ((S)-3-methyl- pyrrolidin-1- ylmethyl)piperidin- 4-ol | 573.3 | | |
| 248 | 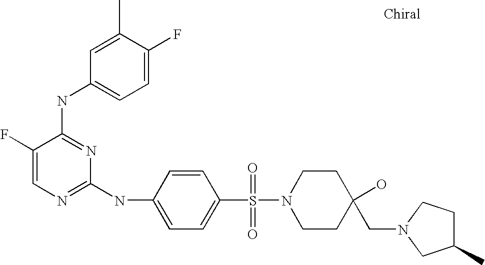 | 1-{4-[5-fluoro-4- (4-fluoro-3-methyl- phenylamino)pyrimidin- 2-ylamino]benzene sulphonyl}-4- ((R)-3-methyl- pyrrolidin-1- ylmethyl)piperidin- 4-ol | 573.3 | | |
| 249 | 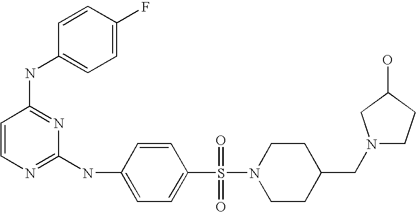 | 1-(1-{4-[4-(4- fluorophenylamino) pyrimidin-2-ylamino] benzenesulphonyl} piperidin-4-ylmethyl) pyrrolidin-3-ol | 527.1 | | |
| 250 | 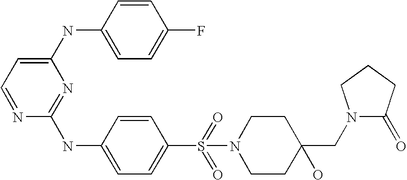 | 1-(1-{4-[4-(4-fluoro- phenylamino) pyrimidin-2-ylamino] benzenesulphonyl}-4- hydroxy-piperidin-4- ylmethyl)pyrrolidin- 2-one | 541.1 | | |
| 251 | 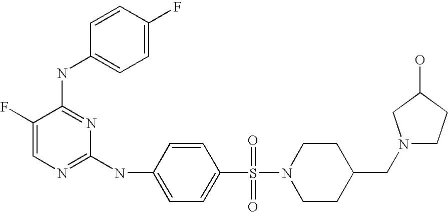 | 1-(1-{4-[5-fluoro- 4-(4-fluorophenyl- amino)pyrimidin-2- ylamino]benzene sulphonyl}piperidin-4- ylmethyl)pyrrolidin- 3-ol | 545 | | |
| 252 | 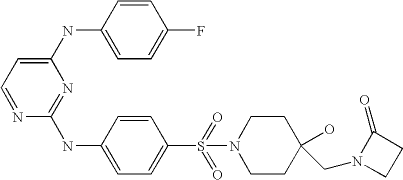 | 1-(1-{4-[4-(4-fluoro- phenylamino) pyrimidin-2-ylamino] benzenesulphonyl} piperidin-4-ylmethyl) azetidin-2-one | | | |
| 253 | 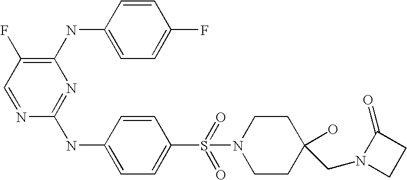 | 1-(1-{4-[5-fluoro4- (4-fluorophenylamino) pyrimidin-2-ylamino] benzenesulphonyl} piperidin-4-ylmethyl) azetidin-2-one | | | |
| 254 | 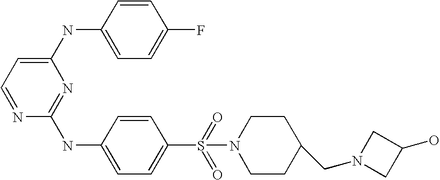 | 1-(1-{4-[4-(4-fluoro- phenylamino) pyrimidin-2-ylamino] benzenesulphonyl} piperidin-4-ylmethyl) azetidin-3-ol | | | |
| 255 | 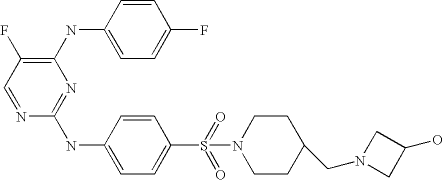 | 1-(1-{4-[5-fluoro- 4-(4-fluorophenyl- amino)pyrimidin-2- ylamino]benzene sulphonyl}piperidin-4- ylmethyl)azetidin-3-ol | | | |
| 256 | 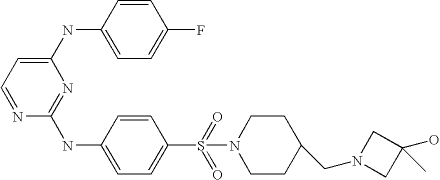 | 1-(1-{4-[4-(4-fluoro- phenylamino) pyrimidin-2-ylamino] benzenesulphonyl} piperidin-4-ylmethyl)- 3-methylazetidin-3-ol | | | |
| 257 | 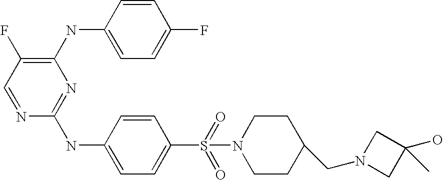 | 1-(1-{4-[4-(5-fluoro- 4-(4-fluorophenyl- amino)pyrimidin-2- ylamino]benzene sulphonyl}piperidin-4- ylmethyl)-3-methyl- azetidin-3-ol | | | |
| 258 | 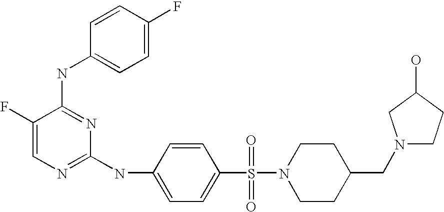 | 1-(1-{4-[5-fluoro- 4-(4-fluorophenyl- amino)pyrimidin-2- ylamino]benzene sulphonyl}piperidin-4- ylmethyl)pyrrolidin- 3-R-ol | | | |
| 260 | 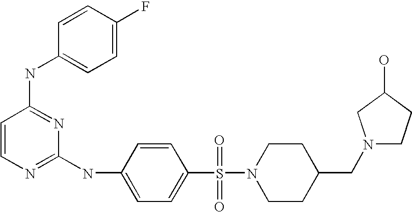 | 1-(1-{4-[4-(4-fluoro- phenylamino) pyrimidin-2-ylamino] benzenesulphonyl} piperidin-4-ylmethyl) pyrrolidin-3-S-ol | | | |

### Example 261

### Pharmaceutical Composition

Tablets corresponding to the following formula were prepared:

| | |
|---|---|
| Product of Example 6 | 0.2 g |
| Excipient for a tablet with a final mass of | 1 g |
| (excipients in detail: lactose, talc, starch, | |
| magnesium stearate). | |

Examples 6 and 105 are taken as examples in the pharmaceutical preparations which constitute Examples 108 and 109 above, it being possible for this pharmaceutical preparation to be prepared differently as indicated above and, if desired, with other products in examples in the present application.

Pharmacological Section:

Protocols for Biochemical Assays on IKK

I) Evaluation of the Compounds on IKK1 and IKK2:

The compounds are tested with respect to the inhibition of IKK1 and IKK2 using a kinase assay on a flash-plate support. The test compounds are dissolved at 10 mM in DMSO and then diluted in kinase buffer (50 mM Tris, pH 7.4, containing 0.1 mM EGTA, 0.1 mM sodium orthovanadate and 0.1% of p-mercaptoethanol).

Serial 3-fold dilutions are prepared using this solution. 10 μl of each dilution are added to the wells of a 96-well plate in duplicate. 10 μl of kinase buffer are added to the control wells which will serve as 0% inhibition and 10 μl of 0.5 mM EDTA are added to the (100% inhibition) control wells. 10 μl of the mixture IKK1 or IKK2 (0.1 μg/well), biotinylated IKB peptide 25-55 substrate and BSA (5 μg) are added to each well. To initiate the kinase reaction, 10 μl of the mixture of 10 mM magnesium acetate, 1 μM cold ATP and 0.1 μCi ³³P-ATP are added to each well for a final volume of 30 μl. The reaction is incubated at 30° C. for 90 min and then stopped by adding 40 μl of 0.5 mM EDTA. After agitation, 50 μl are transferred to a streptavidin-coated flash-plate.

30 min later, the wells are washed twice with a solution of 50 mM Tris-EDTA, pH 7.5, and the radioactivity is determined on a microbeta counter.

The compounds of the invention tested in this assay show an IC50 of less than 10 μM, which shows that they can be used for their therapeutic activity.

II) Evaluation of the Compounds on Tumor Cell Viability and Proliferation:

The compounds according to the invention were subjected to pharmacological assays for determining their anticancer activity.

The compounds of formula (I) according to the present invention were tested in vitro on a panel of tumor lines of human origin, originating:
from breast cancer: MDA-MB231 (American Type culture collection, Rockville, Md., USA, ATCC-HTB26), MDA-A1 or MDA-ADR (referred to as multidrug resistant line MDR, and described by E. Collomb et al., in Cytometry, 12(1):15-25, 1991), and MCF7 (ATCC-HTB22),
from prostate cancer: DU145 (ATCC-HTB81) and PC3 (ATCC-CRL1435),
from colon cancer: HCT116 (ATCC-CCL247) and HCT15 (ATCC-CCL225),
from lung cancer: H460 (described by Carmichael in Cancer Research 47 (4):936-942, 1987 and provided by the National Cancer institute, Frederick Cancer Research and Development Center, Frederick, Md., USA),
from glioblastoma (SF268 described by Westphal in Biochemical & Biophysical Research Communications 132 (1): 284-289, 1985 and provided by the National Cancer institute, Frederick Cancer Research and Development Center, Frederick, Md., USA),
from leukemia (CMLT1 described by Kuriyama et al., in Blood, 74: 1989, 1381-1387, by Soda et al., in British Journal of Haematology, 59: 1985, 671-679 and by Drexler, in Leukemia Research, 18: 1994, 919-927 and provided by the company DSMZ, Mascheroder Weg 1b, 38124 Braunschweig, Germany).

The cell proliferation and viability were determined in a test using 3-(4,5-dimethylthiazol-2-yl)-5-(3-carboxymethoxyphenyl)-2-(4-sulphophenyl)-2H-tetrazolium (MTS) according to T. Fujishita et al., Oncology, 2003, 64 (4), 399-406. In this test, the mitochondrial capacity of living cells to convert MTS to a colored compound after incubation for 72 hours of a compound of formula (I) according to the invention is tested. The concentrations of compound according to the invention which result in a 50% loss of cell proliferation and viability (IC50) are less than 10 μM, depending on the tumor line and the compound tested.

Thus, according to the present invention, it appears that the compounds of formula (I) bring about a loss of tumor cell proliferation and viability with an IC50 of less than 10 μM.

What is claimed is:

1) A compound of formula (I): 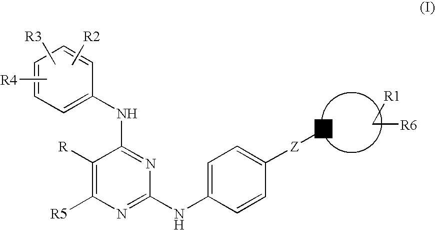
wherein:
R represents a hydrogen or halogen atom;
R2, R3 and R4, which may be identical or different, are such that one of R2, R3, and R4 represents a halogen atom or CF₃ and the other two, which may be identical or different, represent a hydrogen atom or a halogen atom or an alkyl radical or an alkoxy radical optionally substituted with one or more halogen atoms;
R5 represents a hydrogen atom or a halogen atom;
Z represents CO or SO₂;
the ring (N): 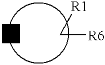
is substituted on the same carbon atom with R1 and R6, contains 4 to 7 ring members, is saturated and optionally contains a carbon-based bridge consisting of 1 to 3 carbons;
R1 and R6 represent one of the following 6 alternatives i) to vi):
i) R1 represents —X1-R7, wherein X1 represents —(CH₂)ₘ— and R7 represents a heterocycloalkyl, aryl or heteroaryl ring, all optionally substituted;
and R6 represents a hydrogen atom, or a hydroxyl, methyl, methoxy, —(CH₂)ₘOH, —CO—NRaRb, —CH₂—NRaRb, —CO₂H or —CO₂alk radical;
ii) R1 represents —X2-R7, wherein X2 represents: —O—, —O—(CH₂)ₘ—, —CH(OH)—(CH₂)ₙ—, —CO—, —CO—NRc-, —CO—NRc-O—, —CH(NRaRb)—, —C═NOH—, —C═N—NH₂—, or —(CH₂)ₙ₁—NRc-(CH₂)ₙ₂—; and
wherein R7 represents a heterocycloalkyl, aryl or heteroaryl ring, all optionally substituted;
and R6 represents hydrogen or the methyl radical;
iii) R1 represents —NRc-W, wherein W represents a hydrogen atom or an alkyl radical containing from 1 to 4 carbon atoms which is linear or branched from 3 carbon atoms and optionally substituted with a radical chosen from —PO(OEt)₂, —OH, —Oalk, —CF₃, —CO—NR8R9 and SO₂-alk; and R6 represents hydrogen;
and wherein when W represents a hydrogen atom, then z represents CO;
iv) R1 represents —CH₂—NRc-W, wherein W represents a hydrogen atom or an alkyl radical containing from 1 to 4 carbon atoms which is linear or branched from 3 carbon atoms and optionally substituted with a radical chosen from —PO(OEt)₂, —OH, —OEt, —CF₃, —CO—N(alk)₂ and SO₂-alk; and R6 represents hydrogen;
v) R1 represents —CO—N(Rc)-OR′c and R6 represents hydrogen; or
vi) R1 represents X3-R7, wherein X3 represents —CH(OH)—(CH₂)ₙ—, —CO—, —CH(NRaRb)—, —C═NOH—, or —C═N—NH₂—;
and wherein R7 represents a heterocycloalkyl, aryl or heteroaryl ring, all optionally substituted;
and R6 represents a hydrogen atom or a hydroxyl, methyl, methoxy, —(CH₂)ₘOH, —CO—NRaRb, —CH₂—NRaRb or —CO₂alk radical;
n, n1 and n2, which may be identical or different, represent an integer from 0 to 3;
m represents an integer from 1 to 3;
Rc and R′c, which may be identical or different, represent a hydrogen atom or an alkyl radical containing from 1 to 4 carbon atoms, optionally substituted with one or more halogen atoms;
NRaRb is such that either Ra and Rb, which may be identical or different, represent a hydrogen atom or an alkyl radical containing from 1 to 4 carbon atoms or a cycloalkyl radical, the alkyl and cycloalkyl radicals being optionally substituted with one or more halogen atoms, a hydroxyl radical or an NH₂, NHalkyl or N(alkyl)₂ radical;
or Ra and Rb form, with the nitrogen atom to which they are attached, a cyclic amine that may optionally contain one or two other heteroatoms chosen from O, S, N or NR10, the cyclic amine thus formed itself being optionally substituted with one or more radicals, which may be identical or different, chosen from halogen atoms and the radicals oxo, hydroxyl, and alkyl, themselves optionally substituted with one or more halogen atoms, or with a methyl radical or a hydroxyl radical on the same carbon; and wherein
all of the heterocycloalkyl, aryl and heteroaryl radicals being optionally substituted with one or more radicals, which may be identical or different, chosen from halogen atoms, hydroxyl, cyano or NR8R9 radicals; and the radicals alkyl, cycloalkyl, alkoxy, phenyl, heterocycloalkyl and heteroaryl, themselves optionally substituted with one or more radicals, which may be identical or different, chosen from halogen atoms and the radicals hydroxyl, alkoxy, alkyl, hydroxyalkyl, alkoxyalkyl, CN, CF₃, OCF₃ or NRaRb;
R8 represents a hydrogen atom or an alkyl radical containing from 1 to 4 carbon atoms or a cycloalkyl radical, these alkyl and cycloalkyl radicals being optionally substituted with one or more halogen atoms, a hydroxyl radical or an NH₂, NHalkyl or N(alkyl)₂ radical;
R9 represents a hydrogen atom or the radicals alkyl, cycloalkyl or heterocycloalkyl, themselves optionally substituted with one or more radicals, which may be identical or different, chosen from halogen atoms and the radicals hydroxyl, alkoxy, NH₂, NHalkyl or N(alkyl)₂, the alkyl radicals represented by R9 also being optionally substituted with a phenyl, heterocycloalkyl or heteroaryl radical, itself optionally substituted with one or more radicals chosen from halogen atoms and the radicals hydroxyl, alkoxy, alkyl, hydroxyalkyl, alkoxyalkyl, CN, CF₂, OCF₂, NH₂, NHalk and N(alk)₂;
or R8 and R9 form, with the nitrogen atom to which they are attached, a cyclic amine that may optionally contain one or two other heteroatoms chosen from O, S, N or NR10, the cyclic amine thus formed itself being optionally substituted with one or more radicals, which may be identical or different, chosen from halogen atoms and alkyl radicals, themselves optionally substituted with one or more halogen atoms; and wherein
all of the above heterocycloalkyl and heteroaryl radicals are optionally substituted as indicated above, consist of from 4 to 10 ring members and contain from 1 to 3 heteroatoms chosen from O, S, N and NR10; and
R10 represents a hydrogen atom or an alkyl radical;
or an acid addition salt thereof.

2) The compound of formula (I) according to claim 1, wherein
R represents a halogen atom;
or an acid addition salt thereof.

3) The compound of formula (I) according to claim 1, wherein
R represents a hydrogen atom;
or an acid addition salt thereof.

4) The compound formula (I) according to claim 1, wherein:
R1 and R6 are as defined in one of the alternatives i) to v);
NRaRb is such that either Ra and Rb, which may be identical or different, represent a hydrogen atom or an alkyl radical containing from 1 to 4 carbon atoms or a cycloalkyl radical, the alkyl and cycloalkyl radicals being optionally substituted with one or more halogen atoms, a hydroxyl radical or an NH₂, NHalkyl or N(alkyl)₂ radical;
or Ra and Rb form, with the nitrogen atom to which they are attached, a cyclic amine that may optionally contain one or two other heteroatoms chosen from O, S, N or NR10, the cyclic amine thus formed itself being optionally substituted with one or more radicals, which may be identical or different, chosen from halogen atoms and alkyl radicals, themselves optionally substituted with one or more halogen atoms;
or an addition salt thereof.

5) The compound of formula (I) according to claim 1, wherein:
R1 represents —X1-R7, wherein X1 represents —(CH₂)ₘ— and R7 represents a heterocycloalkyl, aryl or heteroaryl ring, all optionally substituted;
and R6 represents a hydrogen atom or a hydroxyl, —(CH₂)ₘOH, —CO—NRaRb, —CH₂—NRaRb, —CO₂H, or —CO₂alk radical;
and m, n and NRaRb are as defined in claim 1;
or an acid addition salt thereof.

6) The compound of formula (I) according to claim 1, wherein:
R1 represents —X2-R7, wherein X2 represents —O—, —O—(CH₂)ₘ—, —CH(OH)—(CH₂)ₙ—, —CO—, —CO—NRc-, —CO—NRc-O—, —CH(NRaRb)—, —C═NOH—, —C═N—NH₂—, or —(CH₂)ₙ₁—NRc-(CH₂)ₙ₂—; and wherein R7 represents a heterocycloalkyl, aryl or heteroaryl ring, all optionally substituted;
R6 represents hydrogen;
and n, n1, n2, Rc and NRaRb are as defined in claim 1;
or an acid addition salt thereof.

7) The compound of formula (I) according to claim 1, wherein either R1 represents —NRc-W, wherein W represents a hydrogen atom or an alkyl radical containing from 1 to 4 carbon atoms which is linear or branched from 3 carbon atoms and optionally substituted with a radical chosen from —PO(OEt)₂, —OH, —Oalk, —CF₃, —CO—NR8R9 and SO₂-alk, and R6 represents hydrogen, wherein when W represents a hydrogen atom, then z represents CO;
or R1 represents —CH₂—NRc-W, wherein W represents a hydrogen atom or an alkyl radical containing from 1 to 4 carbon atoms which is linear or branched from 3 carbon atoms and optionally substituted with a radical chosen from —PO(OEt)₂, —OH, —OEt, —CF₃, —CO—N(alk)₂ and SO₂-alk, and R6 represents hydrogen;
or R1 represents —CO—N(Rc)-OR′c and R6 represents hydrogen;
wherein Rc, R′c and NR8R9 are as defined in claim 1,
or an addition salt thereof.

8) A compound of formula (I) according to claim 1, wherein:
the ring (N) represents:
an azetidinyl or pyrrolidinyl ring substituted in the 3-position with R1 and R6, wherein R1 and R6 are as defined in claim 1;
a piperidinyl or azepinyl ring substituted in the 3- or 4-position with R1 and R6, wherein R1 and R6 are as defined in claim 1; or
a 8-azabicyclo(3,2,1)octan-3-yl, 6-azabicyclo[3.2.1]octan-3-yl or 3-azabicyclo[3.2.1]octan-8-yl ring;
or an acid addition salt thereof.

9) The compound of formula (I) according to claim 1, wherein the ring (N) represents a pyrrolidinyl ring substituted in the 3-position with R1 and R6 or the ring (N) represents a piperidinyl ring substituted in the 3- or 4-position with R1 and R6, wherein R1 and R6 are as defined in claim 1;
or an acid addition salt thereof.

10) The compound of formula (I) according to claim 1, wherein:
the ring (N): 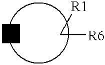
represents a pyrrolidinyl radical substituted in the 3-position with R1 and R6 or a piperidinyl ring substituted in the 3- or 4-position with R1 and R6,
wherein R1 and R6 represent one of the following 5 alternatives i) to v):
i) R1 represents —X1-R7, wherein X1 represents —CH₂ and R7 represents a heterocycloalkyl, phenyl or heteroaryl ring, all optionally substituted;
and R6 represents a hydrogen atom or a the hydroxyl, —CH₂OH, —CO—NRaRb or —CO₂Et radical;
ii) R1 represents —X2-R7, wherein X2 represents: —O—, —CH(OH)—, —CH(OH)—CH₂—, —CO—, —CH(NRaRb)—, —C═NOH—, —C═N—NH₂—, or —(CH₂)ₙ₁—NRc-(CH₂)ₙ₂—;
and wherein R7 represents a heterocycloalkyl, phenyl or heteroaryl ring, all optionally substituted,
and R6 represents hydrogen;
iii) R1 represents —NRc-W, wherein W represents a hydrogen atom or an alkyl radical containing from 1 to 4 carbon atoms which is linear or branched and optionally substituted with a radical chosen from —PO(OEt)₂, —OH, —OEt, —CF₃, —CO—NR8R9 and SO₂-alk;
and R6 represents hydrogen;
wherein when W represents a hydrogen atom, then z represents CO;
iv) R1 represents —CH₂—NRc-W, wherein W represents a hydrogen atom or an alkyl radical containing from 1 to 4 carbon atoms which is linear or branched from 3 carbon atoms and optionally substituted with an SO₂-alk radical; and R6 represents hydrogen; or
v) R1 represents —CO—N(Rc)-OR′c and R6 represents hydrogen;
n, n1 and n2, which may be identical or different, representing an integer from 0 to 2;
Rc and R′c, which may be identical or different, represent a hydrogen atom or an alkyl radical containing 1 or 2 carbon atoms;
NRaRb is such that either Ra and Rb, which may be identical or different, represent a hydrogen atom or an alkyl radical containing from 1 to 4 carbon atoms, optionally substituted with one or more halogen atoms, a hydroxyl radical or an NH₂, NHalkyl or N(alkyl)₂ radical;
or Ra and Rb form, with the nitrogen atom to which they are attached, a morpholinyl or pyrrolidinyl radical optionally substituted with one or more radicals, which may be identical or different, chosen from halogen atoms and alkyl radicals, themselves optionally substituted with one or more halogen atoms; and wherein
all of the heterocycloalkyl, phenyl and heteroaryl radicals being optionally substituted with one or more radicals, which may be identical or different, chosen from halogen atoms; the radicals hydroxyl, cyano or NR8R9; and the radicals alkyl, cycloalkyl, alkoxy, phenyl, heterocycloalkyl and heteroaryl, themselves optionally substituted with one or more radicals, which may be identical or different, chosen from halogen atoms and the radicals hydroxyl, alkoxy, CH₃, —CH₂OH, CN, CF₃, OCF₃ or NRaRb;
R8 represents a hydrogen atom, a linear or branched alkyl radical containing at most 4 carbon atoms or a cycloalkyl radical containing from 3 to 6 ring members, alkyl and cycloalkyl themselves optionally substituted with one or more halogen atoms or a hydroxyl radical;
R9 represents a hydrogen atom or an alkyl radical optionally substituted with one or more radicals, which may be identical or different, chosen from halogen atoms, and the radicals hydroxyl, alkoxy, NH₂, NHalkyl, N(alkyl)₂, phenyl, heterocycloalkyl and heteroaryl, themselves optionally substituted with one or more radicals chosen from halogen atoms and the radicals hydroxyl, OCH₃, CH₃, —CH₂OH, CN, CF₃, OCF₃, NH₂, NHalk and N(alk)₂;
or R8 and R9 form, with the nitrogen atom to which they are attached, a cyclic amine chosen from pyrrolyl, piperidyl, morpholinyl, pyrrolidinyl, azetidinyl and piperazinyl, optionally substituted with one or more alkyl radicals, themselves optionally substituted with one or more halogen atoms;
or an addition salt thereof.

11) The compound formula (I) according to claim 1, wherein:
R2, R3 and R4, which may be identical or different, are such that one or R2, R3, and R4 represents a halogen atom or CF₃ and the other two, which may be identical or different, represent a hydrogen atom, a halogen atom or a methyl, methoxy, trifluoromethyl or trifluoromethoxy radical;
and R5 represents a hydrogen atom;
or an addition salt thereof.

12) The compound of formula (I) according to claim 1, wherein:
R2, R3 and R4, which may be identical or different, are such that one of R2, R3, and R4 represents a fluorine atom and the other two, which may be identical or different, represent a hydrogen atom, a fluorine atom or a methyl radical; and
R5 represents a hydrogen atom;
or an acid addition salt thereof.

13) The compound of formula (I) according to claim 1, wherein
Z represents SO₂;
or an acid addition salt thereof.

14) The compound of formula (I) according to claim 1, wherein
Z represents CO;
or an acid addition salt thereof.

15) The compound of formula (I) according to claim 1, wherein:
either R1 represents —X1-R7, wherein X1 represents —CH₂—, and R6 represents a hydrogen atom or hydroxyl, CH₂—OH, —CO—N(CH₃)₂, —CO—NHCH₃, —CO—NH—(CH₂)₂—N(CH₃)₂ or —CO₂Et radicals;
or R1 represents —X2-R7, wherein X2 represents —O—, —CHOH—, —CH(OH)—CH₂—, —CO—, —CHNH₂—, —NH—CH₂—, —N(CH₃)—CH₂— or CH₂—NH—CH₂—, and R6 represents hydrogen;
R7 is chosen from pyrrolidinyl, piperidinyl, piperazinyl, pyrimidinyl, morpholinyl, thiomorpholinyl, tetrahydrofuran, phenyl, pyridyl, thienyl, thiazolyl, dithiazolyl, pyrazolyl, pyrazinyl, furyl, imidazolyl, pyrrolyl, oxazolyl, isoxazolyl, benzodihydrofuranyl, benzoxodiazolyl, benzothiodiazolyl, benzothienyl, quinolyl, and isoquinolyl radicals;
all the radicals represented by R7 are optionally substituted with one or more radicals, which may be identical or different, chosen from halogen atoms and the radicals hydroxyl, methyl, methoxy, hydroxymethyl, alkoxymethyl, cyano, NH₂, NHalk, N(alk)₂, —CH₂—NH₂, —CH₂—NHalk, —CH₂—N(alk)₂, phenyl, morpholinyl and CH2-morpholinyl, themselves optionally substituted with one or more radicals, which may be identical or different, chosen from halogen atoms and the radicals hydroxyl, CH₃, OCH₃, —CH₂OH, CN, CF₃, OCF₃, NH₂, NHalk and N(alk)₂;
or an acid addition salt thereof.

16) The compound of formula (I) according to claim 1, selected from the group consisting of:
{4-[4-(4-fluorophenylamino)pyrimidin-2-ylamino]phenyl}-[4-(methyloxazol-2-ylmethylamino)piperidin-1-yl]methanone;
{4-[4-(4-fluorophenylamino)pyrimidin-2-ylamino]phenyl}-[3-(methyl-1H-pyrrole-2-ylmethylamino)piperidin-1-yl]methanone;
1-{4-[4-(4-fluorophenylamino)pyrimidin-2-ylamino]-benzoyl}-3-pyridin-3-ylmethylpiperidine-3-carboxylic acid methylamide;
N-4-(4-fluoro-3-methylphenyl)-N-2-(4-{3-[(2-methanesulphonylethylamino)methyl]pyrrolidine-1-sulphonyl}-phenyl)pyrimidine-2,4-diamine;
N-4-(4-fluoro-3-methylphenyl)-N-2-[4-(3-{[(1-methyl-1H-pyrrol-2-ylmethyl)amino]methyl}pyrrolidine-1-sulphonyl)phenyl]pyrimidine-2,4-diamine;
4-pyrrolidin-1-ylmethyl-1-{4-[4-(4-fluoro-3-methylphenylamino)pyrimidin-2-ylamino]benzenesulphonyl}-piperidin-4-ol;
{4-[4-(4-fluoro-3-methylphenylamino)pyrimidin-2-ylamino]phenyl}-[4-(methylpyridin-2-ylmethylamino)piperidin-1-yl]methanone;
{4-[4-(4-fluoro-3-methylphenylamino)pyrimidin-2-ylamino]phenyl}-[4-(methylpyridin-4-ylmethylamino)piperidin-1-yl]methanone;
{4-[(1,5-dimethyl-1H-pyrazol-4-ylmethyl)methylamino]piperidin-1-yl}-{4-[4-(4-fluoro-3-methylphenylamino)pyrimidin-2-ylamino]phenyl}methanone;
{4-[(2-aminopyridin-3-ylmethyl)methylamino]piperidin-1-yl}-{4-[4-(4-fluoro-3-methylphenylamino)pyrimidin-2-ylamino]phenyl}methanone;
4-{[(1-{4-[4-(4-fluoro-3-methylphenylamino)pyrimidin-2-ylamino]benzoyl}piperidin-4-yl)methylamino]methyl}-1,5-dimethyl-1H-pyrrole-2-carbonitrile;
{4-[(2,4-dimethylthiazol-5-ylmethyl)methylamino]piperidin-1-yl}-{4-[4-(4-fluoro-3-methylphenylamino)pyrimidin-2-ylamino]phenyl}methanone; and
(1-{[4-({4-[(4-fluorophenyl)amino]pyrimidin-2-yl}amino)phenyl]sulphonyl}piperidin-4-yl)(pyridin-3-yl)-methanamine;
or an addition salt thereof.

17) A process for preparing a compound of formula (Ia): 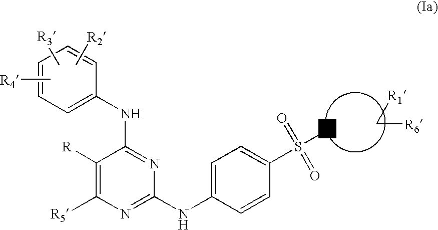
wherein R is as defined in claim 1 for the compounds of formula (I) and R₁′, R₂′, R₃′, R₄′, R₅′ and R₆′ are as defined in claim 1 for R2, R3, R4, R5, and R6 respectively, in which the possible reactive functions are optionally protected,
said process comprising:
reacting a compound of formula (II): 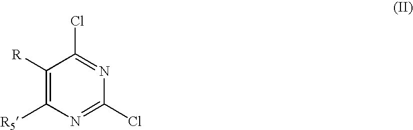
wherein R and R₅′ are as defined above,
with a compound of formula (III): 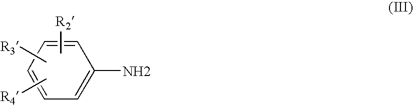
wherein R2′, R3′ and R4′ are as defined above,
so as to obtain a product of formula (IV): 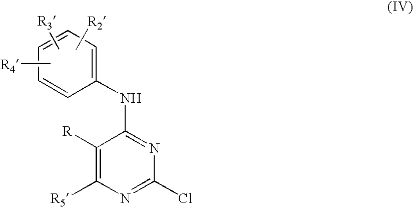
wherein R, R₂′, R₃′, R₄′ and R₅′ are as defined above;
reacting the compound of formula (IV) with the aniline of formula (V): 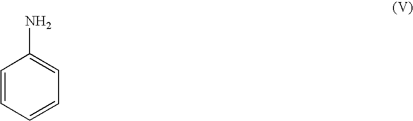
so as to obtain a product of formula (VI): 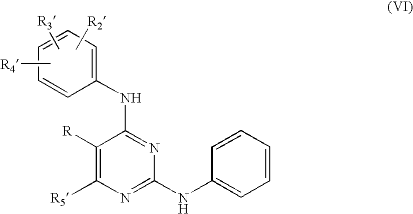
wherein R, R₂′, R₃′, R₄′ and R₅′ are as defined above;
reacting the compound of formula (VI) with chlorosulphonic acid SO₂(OH)Cl so as to obtain the corresponding product of formula (VII): 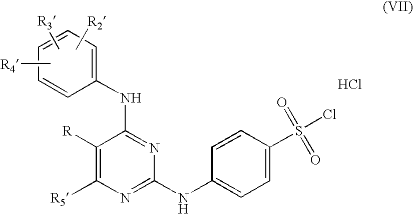
wherein R, R₂′, R₃′, R₄′ and R₅′ are as defined above; and
reacting the compound of formula (VII) with an amine of formula (VIII): 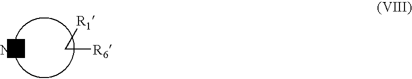
wherein R₁′ and R₆′ are as defined above,
so as to obtain a product of formula (Ia); and
optionally subjecting the compound of formula (Ia) to one or more of the following conversion reactions in any order:
a) a reaction of oxidation of an alkylthio group to the corresponding sulphoxide or sulphone group,
b) a reaction of conversion of an alkoxy function to a hydroxyl function, or else of a hydroxyl function to an alkoxy function,
c) a reaction of oxidation of an alcohol function to an aldehyde or ketone function,
d) a reaction of removal of the protective groups that may be borne by the protected reactive functions,
e) a salification reaction with an inorganic or organic acid so as to obtain the corresponding salt, and
f) a reaction to resolve the racemic forms into resolved products.

18) A process for preparing a compound of formula (Ib): 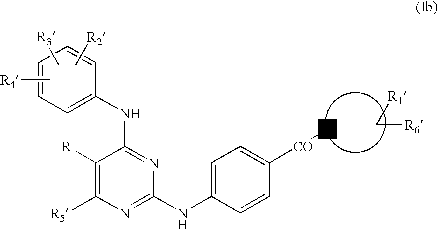
wherein R is as defined in claim 1 for the compound of formula (I), and R₁′, R₂′, R₃′, R₄′, R₅′ and R₆′ are as defined in claim 1 for R2, R3, R4, R5, and R6 respectively, in which the possible reactive functions are optionally protected,
reacting a compound of formula (II): 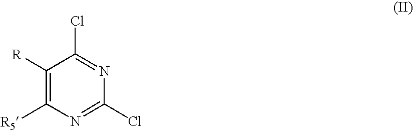
wherein R and R₅′ are as defined above,
with a compound of formula (III): 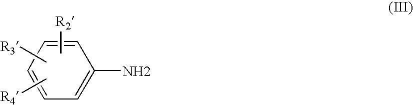
wherein R, R₂′, R₃′, and R₄′ are as defined above,
so as to obtain a product of formula (IV): 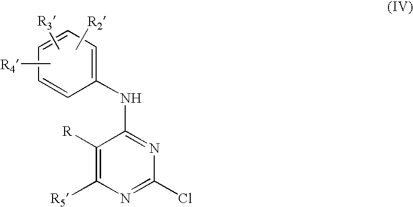
wherein R, R₂′, R₃′, R₄′ and R₅′ are as defined above;
reacting the compound formula (IV) with 4-aminobenzoic acid methyl ester so as to obtain the product of formula (IX): 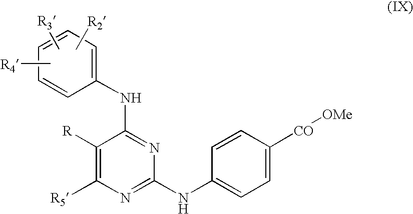
in which R, R₂′, R₃′, R₄′ and R₅′ are as defined above; and
saponifying the compound formula (IX) to its corresponding acid of formula (X): 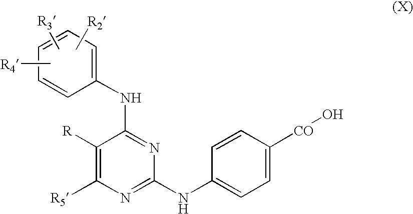
wherein R, R₂′, R₃′, R₄′ and R₅′ are as defined above;
reacting the compound of formula (X) with an amine of formula (VIII) as defined above:
so as to obtain a product of formula (Ib): 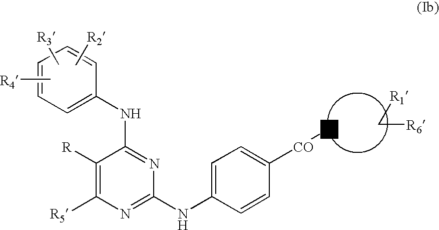
in which R, R₁′, R₂′, R₃′, R₄′, R₅′, and R₆′ are as defined above; and
optionally subjecting the compound of formula (Ib) to one or more of the following conversion reactions in any order:
a) a reaction of oxidation of an alkylthio group to the corresponding sulphoxide or sulphone group,
b) a reaction of conversion of an alkoxy function to a hydroxyl function, or else of a hydroxyl function to an alkoxy function,
c) a reaction of oxidation of an alcohol function to an aldehyde or ketone function,
d) a reaction of removal of the protective groups that may be borne by the protected reactive functions,
e) a salification reaction with an inorganic or organic acid so as to obtain the corresponding salt, and
f) a reaction to resolve the racemic forms into resolved products.

19) A pharmaceutical composition comprising a compound of formula (I) or a pharmaceutically acceptable salt thereof and a pharmaceutically acceptable carrier.

20) A pharmaceutical composition comprising a compound according to claim 16 or a pharmaceutically acceptable salt thereof and a pharmaceutically acceptable carrier.

21) A method of inhibiting the activity of the protein kinase IKK comprising contacting said protein kinase with a compound according to claim 1 or a pharmaceutically acceptable salt thereof.

22) The method according to claim 21, wherein the protein kinase is in a mammal.

23) A method of treating or preventing a disease selected from inflammatory diseases, diabetes and cancer, comprising administering to a patient in need of said treatment or prevention a therapeutically effective amount of compound according to claim 1 or a pharmaceutically acceptable salt thereof.

24) The method according to claim 23 wherein the disease is cancer.

25) The method according to claim 24 wherein the cancer is resistant to cytotoxic agents.
